(19) United States
(12) Patent Application Publication
(10) Pub. No.: US 2017/0197939 A1
(43) Pub. Date: Jul. 13, 2017

(54) Tropomyosin-Related Kinase Inhibitors Containing Both A 1H-Pyrazole And A Pyrimidine Moiety

(71) Applicant: PFIZER INC., New York, NY (US)

(72) Inventors: Sharanjeet Kaur Bagal, Great Abington, Cambridge (GB); Jingrong Jean Cui, San Diego, CA (US); Samantha Elizabeth Greasley, San Diego, CA (US); Elizabeth Ann Lunney, San Diego, CA (US); Indrawan James McAlpine, San Diego, CA (US); Asako Nagata, San Diego, CA (US); Sacha Ninkovic, La Jolla, CA (US); Kiyoyuki Omoto, Great Abington, Cambridge (GB); Sarah Elizabeth Skerratt, Great Abington, Cambridge (GB); Robert Ian Storer, Great Abington, Cambridge (GB); Joseph Scott Warmus, Ledyard, CT (US)

(73) Assignee: Pfizer Inc., New York, NY (US)

(21) Appl. No.: 15/300,440

(22) PCT Filed: Apr. 1, 2015
(86) PCT No.: PCT/IB2015/052414

§ 371 Date: Sep. 29, 2016

Related U.S. Application Data
(60) Provisional application No. 61/979,629, filed on Apr. 15, 2014.

(51) Int. Cl. C07D 401/14; A61K 31/4439; C07D 401/12

(52) U.S. Cl. CPC C07D 401/14; C07D 401/12; A61K 31/4439

(57) ABSTRACT

The present invention relates to compounds of Formula I

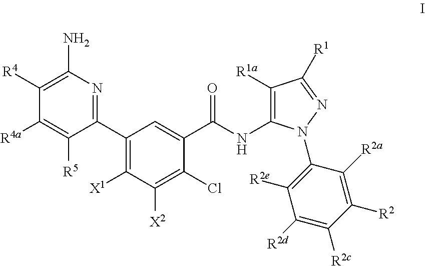

and their prodrugs and pharmaceutically acceptable salts, wherein the substituents are as described herein, and their use in medicine, in particular as TrkA antagonists.

The invention described herein relates to certain heterocyclic compounds and the pharmaceutically acceptable salts of such compounds. The invention also relates to the processes for the preparation of the compounds, compositions containing the compounds, and the uses of such compounds and salts in treating diseases or conditions associated with tropomyosin-related kinase (Trk), activity. More specifically the invention relates to the compounds and their salts useful as inhibitors of Trk, especially TrkA, more especially selective TrkA inhibitors.

### BACKGROUND

Tropomyosin-related kinases (Trks) are a family of receptor tyrosine kinases activated by neurotrophins. Trks play important roles in pain sensation as well as tumour cell growth and survival signaling. Thus, inhibitors of Trk receptor kinases might provide targeted treatments for conditions such as pain and cancer. Recent developments in this field have been reviewed by Wang et al in Expert Opin. Ther. Patents (2009) 19(3): 305-319 and an extract is reproduced below.

### “1.1 Trk Receptors

As one of the largest family of proteins encoded by the human genome, protein kinases are the central regulators of signal transduction as well as control of various complex cell processes. Receptor tyrosine kinases (RTKs) are a subfamily of protein kinases (up to 100 members) bound to the cell membrane that specifically act on the tyrosine residues of proteins. One small group within this subfamily is the Trk kinases, with three highly homologous isoforms: TrkA, TrkB, and TrkC. All three isoforms are activated by high affinity growth factors named neurotrophins (NT): i) nerve growth factor (NGF), which activates TrkA; ii) brain-derived neurotrophic factor (BDNF) and NT-4/5, which activate TrkB; and iii) NT-3, which activates TrkC. The binding of neurotrophins to the extracellular domain of Trks causes the Trk kinase to autophosphorylate at several intracellular tyrosine sites and triggers downstream signal transduction pathways. Trks and neurotrophins are well known for their effects on neuronal growth and survival.

### 1.2 Trks and Cancer

Originally isolated from neuronal tissues, Trks were thought to mainly affect the maintenance and survival of neuronal cells. However, in the past 20 years, increasing evidence has suggested that Trks play key roles in malignant transformation, chemotaxis, metastasis, and survival signaling in human tumors. The association between Trks and cancer focused on prostate cancer in earlier years and the topic has been reviewed. For example, it was reported that malignant prostate epithelial cells secrete a series of neurotrophins and at least one Trks. In pancreatic cancer, it was proposed that paracrine and/or autocrine neurotrophin-Trk interactions may influence the invasive behavior of the cancer. TrkB was also reported to be overexpressed in metastatic human pancreatic cancer cells. Recently, there have been a number of new findings in other cancer settings. For example, a translocation leads to expression of a fusion protein derived from the N-terminus of the ETV6 transcription factor and the C-terminal kinase domain of TrkC. The resulting ETV6-TrkC fusions are oncogenic in vitro and appear causative in secretory breast carcinoma and some acute myelogenous leukemias (AML). Constitutively active TrkA fusions occurred in a subset of papillary thyroid cancers and colon carcinomas. In neuroblastoma, TrkB expression was reported to be a strong predictor of aggressive tumor growth and poor prognosis, and TrkB overexpression was also associated with increased resistance to chemotherapy in neuroblastoma tumor cells in vitro. One report showed that a novel splice variant of TrkA called TrkAIII signaled in the absence of neurotrophins through the inositol phosphate-AKT pathway in a subset of neuroblastoma. Also, mutational analysis of the tyrosine kinome revealed that Trk mutations occurred in colorectal and lung cancers. In summary, Trks have been linked to a variety of human cancers, and discovering a Trk inhibitor and testing it clinically might provide further insight to the biological and medical hypothesis of treating cancer with targeted therapies.

### 1.3 Trks and Pain

Besides the newly developed association with cancer, Trks are also being recognized as an important mediator of pain sensation. Congenital insensitivity to pain with anhidrosis (CIPA) is a disorder of the peripheral nerves (and normally innervated sweat glands) that prevents the patient from either being able to adequately perceive painful stimuli or to sweat. TrkA defects have been shown to cause CIPA in various ethnic groups.

Currently, non-steroidal anti-inflammatory drugs (NSAIDs) and opiates have low efficacy and/or side effects (e.g., gastrointestinal/renal and psychotropic side effects, respectively) against neuropathic pain and therefore development of novel pain treatments is highly desired. It has been recognized that NGF levels are elevated in response to chronic pain, injury and inflammation and the administration of exogenous NGF increases pain hypersensitivity. In addition, inhibition of NGF function with either anti-NGF antibodies or non-selective small molecule Trk inhibitors has been shown to have effects on pain in animal models. It appears that a selective Trk inhibitor (inhibiting at least NGF's target, the TrkA receptor) might provide clinical benefit for the treatment of pain. Excellent earlier reviews have covered targeting NGF/BDNF for the treatment of pain so this review will only focus on small molecule Trk kinase inhibitors claimed against cancer and pain. However, it is notable that the NGF antibody tanezumab was very recently reported to show good efficacy in a Phase II trial against osteoarthritic knee pain.”

Further trk-mediated conditions which have been investigated and show promise for treatment with a trk inhibitor include atopic dermatitis, psoriasis, eczema and prurigo nodularis, acute and chronic itch, pruritis, atopic dermatitis, inflammation, cancer, restenosis, atherosclerosis, psoriasis, thrombosis, pruritis, lower urinary tract disorder, inflammatory lung diseases such as asthma, allergic rhinitis, lung cancer, psoriatic arthritis, rheumatoid arthritis, inflammatory bowel diseases such as ulcerative colitis, Crohn's disease, fibrosis, neurodegenerative disease, diseases disorders and conditions related to dysmyelination or demyelination, certain infectious diseases such as Trypanosome cruzi infection (Chagas disease), cancer related pain, chronic pain, neuroblastoma, ovarian cancer, colorectal cancer, melanoma, head and neck cancer, gastric carcinoma, lung carcinoma, breast cancer, glioblastoma, medulloblastoma, secretory breast cancer, salivary gland cancer, papillary thyroid carcinoma, adult myeloid leukaemia, tumour growth and metastasis and interstitial cystitis (C. Potenzieri and B. J. Undem, Clinical & Experimental Allergy, 2012 (42) 8-19; Yamaguchi J, Aihara M, Kobayashi Y, Kambara T, Ikezawa Z, J Dermatol Sci. 2009; 53:48-54; Dou Y C, Hagstromer L, Emtestam L, Johansson O., Arch Dermatol Res. 2006; 298:31-37; Johansson O, Liang Y, Emtestam L., Arch Dermatol Res. 2002; 293:614-619; Grewe M, Vogelsang K, Ruzicka T, Stege H, Krutmann J., J Invest Dermatol. 2000; 114:1108-1112; Urashima R, Mihara M. Virchows Arch. 1998; 432:363-370; Kinkelin I, Motzing S, Koltenzenburg M, Brocker E B., Cell Tissue Res. 2000; 302:31-37; Tong Liu & Ru-Rong Ji, Pflugers Arch—Eur J Physiol, DOI 10.1007/s00424-013-1284-2, published online 1 May 2013); International Patent Application publication numbers WO2012/158413, WO2013/088256, WO2013/088257 and WO2013/161919, (Brodeur, G. M., Nat. Rev. Cancer 2003, 3, 203-216), (Davidson. B., et al., Clin. Cancer Res. 2003, 9, 2248-2259), (Bardelli, A., Science 2003, 300, 949), (Truzzi, F., et al., Dermato-Endocrinology 2008, 3 (I), pp. 32-36), Yilmaz, T., et al., Cancer Biology and Therapy 2010, 10 (6), pp. 644-653), (Du, J. et al., World Journal of Gastroenterology 2003, 9 (7), pp. 1431-1434), (Ricci A., et al., American Journal of Respiratory Cell and Molecular Biology 25 (4), pp. 439-446), (Jin, W., et al., Carcinogenesis 2010, 31 (11), pp. 1939-1947), (Wadhwa, S., et al., Journal of Biosciences 2003, 28 (2), pp. 181-188), (Gruber-Olipitz, M., et al., Journal of Proteome Research 2008, 7 (5), pp. 1932-1944), (Euthus, D. M. et al., Cancer Cell 2002, 2 (5), pp. 347-348), (Li, Y.-G., et al., Chinese Journal of Cancer Prevention and Treatment 2009, 16 (6), pp. 428-430), (Greco, A., et al., Molecular and Cellular Endocrinology 2010, 321 (I), pp. 44-49), (Eguchi, M., et al., Blood 1999, 93 (4), pp. 1355-1363), (Nakagawara, A. (2001) Cancer Letters 169:107-114; Meyer, J. et al. (2007) Leukemia, 1-10; Pierottia, M. A. and Greco A., (2006) Cancer Letters 232:90-98; Eric Adriaenssens, E., et al. Cancer Res (2008) 68:(2) 346-351), (Freund-Michel, V; Frossard, N., Pharmacology ck Therapeutics (2008) 117(1), 52-76), (Hu Vivian Y; et. al. The Journal of Urology (2005), 173(3), 1016-21), (Di Mola, F. F, et. al. Gut (2000) 46(5), 670-678) (Dou, Y.-C., et. al. Archives of Dermatological Research (2006) 298(1), 31-37), (Raychaudhuri, S. P., et al., J. Investigative Dermatology (2004) 122(3), 812-819) and (de Melo-Jorge, M. et al., Cell Host ck Microbe (2007) 1(4), 251-261).

International Patent Application publication number WO2009/012283 refers to various fluorophenyl compounds as Trk inhibitors; International Patent Application publication numbers WO2009/152087, WO2008/080015 and WO2008/08001 and WO2009/152083 refer to various fused pyrroles as kinase modulators; International Patent Application publication numbers WO2009/143024 and WO2009/143018 refer to various pyrrolo[2,3-d]pyrimidines substituted as Trk inhibitors; International Patent Application publication numbers WO2004/056830 and WO2005/116035 describe various 4-amino-pyrrolo[2,3-d]pyrimidines as Trk inhibitors. International Patent Application publication number WO2011/133637 describes various pyrrolo[2,3-d]pyrimidines and pyrrolo[2,3-b]pyridines as inhibitors of various kinases. International Patent Application publication number WO2005/099709 describes bicyclic heterocycles as serine protease inhibitors. International Patent Application publication number WO2007/047207 describes bicyclic heterocycles as FLAP modulators. International Patent Application publication number WO2012/158413 describes pyrrolidinyl urea and pyrrolidinyl thiourea compounds as trkA kinase inhibitors. International Patent Application publication number WO2010/077680 describes compounds with a bicyclic core as trkA kinase inhibitors.

Thus Trk inhibitors have a wide variety of potential medical uses. There is a need to provide new Trk inhibitors that are good drug candidates. In particular, compounds should preferably bind potently to the Trk receptors in a selective manner compared to other receptors, whilst showing little affinity for other receptors, including other kinase and/or GPC receptors, and show functional activity as Trk receptor antagonists. They should be non-toxic and demonstrate few side-effects. Furthermore, the ideal drug candidate will exist in a physical form that is stable, non-hygroscopic and easily formulated. They should preferably be e.g. well absorbed from the gastrointestinal tract, and/or be injectable directly into the bloodstream, muscle, or subcutaneously, and/or be metabolically stable and possess favourable pharmacokinetic properties.

Among the aims of this invention are to provide orally-active, efficacious, compounds and salts which can be used as active drug substances, particularly Trk antagonists, i.e. that block the intracellular kinase activity of the Trk, e.g. TrkA (NGF) receptor. Other desirable features include selectivity for TrkA vs other Trk receptors (e.g. B and/or C), good HLM/hepatocyte stability, oral bioavailability, metabolic stability, absorption, selectivity over other types of kinase, dofetilide selectivity. Preferable compounds and salts will show a lack of CYP inhibition/induction, and be CNS-sparing.

### SUMMARY

The present invention provides compounds of Formula I

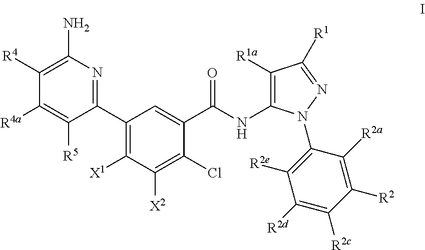

and prodrugs thereof, and pharmaceutically acceptable salts thereof, wherein
R¹ is CON(H or C₁₋₆ alkyl optionally substituted by 1 or 2 substituents independently selected from F, OH and OMe)(H or C₁₋₆ alkyl optionally substituted by 1, 2 or 3 substituents independently selected from F, NH₂, OH and OMe), CONR^{x1}(C₃₋₆cycloalkyl optionally substituted by 1, 2 or 3 substituents independently selected from OH, NH₂, CH₃ and CH₂OH), CONR^{x1}(CR^{Y}R^{X})ₘ(C₃₋₆ cycloalkyl optionally substituted by 1, 2 or 3 substituents independently selected from OH, NH₂, CH₃ and CH₂OH), CONR^{x1}-Het, CO—NHet, CONR^{x1}(CR^{Y}R^{X})ₘ—CON(H or C₁₋₄ alkyl optionally substituted by 1 or 2 substituents independently selected from F, OH and OMe)(H or C₁₋₄ alkyl optionally substituted by 1, 2 or 3 substituents independently selected from F, OH and OMe), CONR^{x1}(CR^{Y}R^{X})ₘ—N(H or C₁₋₄ alkyl optionally substituted by 1 or 2 substituents independently selected from F, OH and OMe)(H or C₁₋₄ alkyl optionally substituted by 1, 2 or 3 substituents independently selected from F, OH and OMe), CONR^{x1}(CR^{Y}R^{X})ₘN(H or C₁₋₄ alkyl optionally substituted by 1 or 2 substituents independently selected from F, OH and OMe)CO(C₁₋₄ alkyl optionally substituted by 1, 2 or 3 substituents independently selected from F, OH and OMe), CONR^{x1}C(O)-Het, C(O)NR^{x1}(CR^{Y}R^{X})ₘ-Het, CONR^{x1}—Ar, C(O)—NR^{x1}-Het, CN, CO₂H, or CO₂(C₁₋₄ alkyl optionally substituted by 1 or 2 substituents independently selected from F, OH and OMe),
m is an integer from 1 to 3,
Ar is phenyl optionally substituted by 1, 2 or 3 groups independently selected from C₁₋₆ alkyl, halogen, CN, CF₃, CF₃O, C₁₋₆ alkoxy, C₁₋₆ alkoxy-O—C(O)—, CONH₂, C₁₋₆ alkylthio, hydroxy-C₁₋₆ alkyl, C₁₋₆ alkyl-SO₂—, CO₂H and C₁₋₃ alkoxy-C₁₋₃ alkyl-O—C(O)—,
Het is a 4-7-membered saturated or unsaturated heterocyclic ring having at least 1, and up to 3, hetero ring atoms independently selected from N, O and S, and which ring is optionally substituted by 1, 2 or 3 substituents independently selected from halogen, OH, ═O, CN, CONH₂, O(C₁₋₆ alkyl optionally substituted by one or more F), C(O)(C₁₋₆ alkyl optionally substituted by one or more F), C₁₋₆ alkyl optionally substituted by one or more F, C₁₋₆ alkyl substituted by CN, C₁₋₆ alkyl substituted by up to 3 OH, C₁₋₆ alkyl substituted by CO₂(C₁₋₄ alkyl), C₁₋₆ alkyl substituted by one or more C₁₋₃ alkoxy, S(O)ₚ(C₁₋₆ alkyl optionally substituted by one or more F), CO₂(C₁₋₆ alkyl), C₃₋₆ cycloalkyl, C(O)(C₃₋₆ cycloalkyl), N(H or C₁₋₃ alkyl)CO(C₁₋₃ alkyl) and N(H or C₁₋₃ alkyl)(H or C₁₋₃ alkyl),
NHet is a 4-7-membered saturated or unsaturated heterocyclic ring with a ring N atom directly linked to the C(O) moiety, having from 0 to 2 further hetero ring atoms independently selected from N, O and S, and which ring is optionally substituted by up to 3 substituents independently selected from halogen, OH, ═O, CN, CONH₂, C₁₋₆ alkyl optionally substituted by one or more F, O(C₁₋₆ alkyl optionally substituted by one or more F), C(O)(C₁₋₆ alkyl optionally substituted by one or more F), C₁₋₆ alkyl substituted by CN, C₁₋₆ alkyl substituted by up to 3 OH, C₁₋₆ alkyl substituted by CO₂(C₁₋₄ alkyl), C₁₋₆ alkyl substituted by one or more C₁₋₃ alkoxy, S(O)ₚ(C₁₋₆ alkyl optionally substituted by one or more F), CO₂(C₁₋₆ alkyl), C₃₋₆ cycloalkyl, C(O)(C₃₋₆ cycloalkyl), N(H or C₁₋₃ alkyl)CO(C₁₋₃ alkyl), and N(H or C₁₋₃ alkyl)(H or C₁₋₃ alkyl),
R^{2a}, R², R^{2c}, R^{2d} and R^{2e} are each independently selected from H, OH, halogen, NH₂, or methyl optionally substituted by up to 3 F,
X¹, X², R^{1a}, R⁴, R^{4a} and R⁵ are each independently selected from H, C₃₋₆ cycloalkyl, C₀₋₆ alkyl optionally substituted by up to 3 substituents independently selected from halogen, CN, CO₂H, OH, (C₁₋₆ alkoxy optionally substituted by up to 3 F), S(O)ₚ(C₁₋₆ alkyl optionally substituted by up to 3 F), C(O)(C₁₋₆ alkoxy optionally substituted by up to 3 F or by C₁₋₃ alkoxy), C(O)NR^{x1}R^{x2}, NR^{x1}R^{x2}, O(C₃₋₆ cycloalkyl), Ar, Het, CO₂(C₁₋₆ alkyl optionally substituted by up to 3 F), NR^{x1}C(O)(C₁₋₆ alkyl optionally substituted by up to 3 F), NR^{x1}C(O)NR^{x2}(C₁₋₆ alkyl optionally substituted by up to 3 F), OC(O)(C₁₋₆ alkyl optionally substituted by up to 3 F), and OC(O)NR^{x1}R^{x2},
p is 0, 1 or 2,
R^{x1} and R^{x2} are each independently H, C₁₋₃ alkyl optionally substituted by up to 3 substituents independently selected from F, OH and OCH₃, or, together with the nitrogen atom to which they are attached, form a 4- to 6-membered saturated ring,
and R^{x} and R^{y} are each independently H, C₁₋₃ alkoxy optionally substituted by up to 3 F, C₁₋₃ alkyl optionally substituted by up to 3 substituents independently selected from F, OH and OCH₃, or, together with the carbon atom to which they are attached, are C₃₋₆ cycloalkyl.

The invention also comprises pharmaceutical compositions comprising a therapeutically effective amount of a compound of formula I as defined herein, or a pharmaceutically acceptable salt thereof, and a pharmaceutically acceptable carrier.

The invention is also directed to a method of treating a disease or condition indicated for treatment with a Trk antagonist, in a subject, by administering to a subject in need thereof a therapeutically effective amount of one or more of the compounds herein, or a pharmaceutically acceptable salt thereof.

Other aspects of the invention will be apparent from the remaining description and claims.

Preferably, the compounds of the present invention are potent antagonists at TrkA receptors, and have a suitable PK profile to enable once daily dosing.

The compounds of the present invention are potentially useful in the treatment of a range of disorders where a TrkA antagonist is indicated, particularly pain indications. Depending on the disease and condition of the patient, the term “treatment” as used herein may include one or more of curative, palliative and prophylactic treatment.

Disorders for which a trkA inhibitor may be indicated include pain. Pain may be either acute or chronic and additionally may be of central and/or peripheral origin. Pain may be of a neuropathic and/or nociceptive and/or inflammatory nature, such as pain affecting either the somatic or visceral systems, as well as dysfunctional pain affecting multiple systems.

Physiological pain is an important protective mechanism designed to warn of danger from potentially injurious stimuli from the external environment. The system operates through a specific set of primary sensory neurones and is activated by noxious stimuli via peripheral transducing mechanisms (see Meyer et al., 2006, Wall and Melzack's Textbook of Pain (5^{th}) Ed), Chapter1). These sensory fibres are known as nociceptors, and are characteristically small diameter axons with slow conduction velocities, of which there are two main types, A-delta fibres (myelinated) and C fibres (non-myelinated). Nociceptors encode the intensity, duration and quality of noxious stimulus and by virtue of their topographically organised projection to the spinal cord, the location of the stimulus. The activity generated by nociceptor input is transferred, after complex processing in the dorsal horn, either directly, or via brain stem relay nuclei, to the ventrobasal thalamus and then on to the cortex, where the sensation of pain is generated.

Pain may generally be classified as acute or chronic. Acute pain begins suddenly and is short-lived (usually twelve weeks or less). It is usually, although not always, associated with a specific cause such as a defined injury, is often sharp and severe and can result from numerous origins such as surgery, dental work, a strain or a sprain. Acute pain does not generally result in any persistent psychological response. When a substantial injury occurs to body tissue, via disease or trauma, the characteristics of nociceptor activation may be altered such that there is sensitisation in the periphery, locally around the injury and centrally where the nociceptors terminate. These effects lead to a heightened sensation of pain. In acute pain these mechanisms can be useful, in promoting protective behaviours which may better enable repair processes to take place. The normal expectation would be that sensitivity returns to normal once the injury has healed. However, in many chronic pain states, the hypersensitivity far outlasts the healing process and is often due to nervous system injury or alteration which can be associated with maladaptation and aberrant activity (Woolf & Salter, 2000, Science, 288, 1765-1768). As such, chronic pain is long-term pain, typically persisting for more than three months and leading to significant psychological and emotional problems. Common examples of chronic pain are neuropathic pain (e.g. painful diabetic neuropathy or postherpetic neuralgia), carpal tunnel syndrome, back pain, headache, cancer pain, arthritic pain and chronic post-surgical pain, but may include any chronic painful condition affecting any system, such as those described by the International Association for the Study of Pain (Classification of Chronic Pain, a publication freely available for download at http://www.iasp-pain.org).

The clinical manifestation of pain is present when discomfort and abnormal sensitivity feature among the patient's symptoms. Patients tend to be quite heterogeneous and may present with various pain symptoms. Such symptoms can include: 1) spontaneous pain which may be dull, burning, or stabbing; 2) exaggerated pain responses to noxious stimuli (hyperalgesia); and 3) pain produced by normally innocuous stimuli (allodynia) (Meyer et al., 2006, Wall and Melzack's Textbook of Pain (5^{th}) Ed), Chapter1). Although patients suffering from various forms of acute and chronic pain may have similar symptoms, the underlying mechanisms may be different and may, therefore, require different treatment strategies. Apart from acute or chronic, pain can also be broadly categorized into: nociceptive pain, affecting either the somatic or visceral systems, which can be inflammatory in nature (associated with tissue damage and the infiltration of immune cells); or neuropathic pain.

Nociceptive pain can be defined as the process by which intense thermal, mechanical, or chemical stimuli are detected by a subpopulation of peripheral nerve fibers, called nociceptors, and can be induced by tissue injury or by intense stimuli with the potential to cause injury. Pain afferents are activated by transduction of stimuli by nociceptors at the site of injury and activate neurons in the spinal cord at the level of their termination. This is then relayed up the spinal tracts to the brain where pain is perceived (Meyer et al., 2006, Wall and Melzack's Textbook of Pain (5^{th}) Ed), Chapter1). Myelinated A-delta fibres transmit rapidly and are responsible for sharp and stabbing pain sensations, whilst unmyelinated C fibres transmit at a slower rate and convey a dull or aching pain. Moderate to severe acute nociceptive pain is a prominent feature of pain from strains/sprains, burns, myocardial infarction and acute pancreatitis, post-operative pain (pain following any type of surgical procedure), posttraumatic pain, pain associated with gout, cancer pain and back pain. Cancer pain may be chronic pain such as tumour related pain (e.g. bone pain, headache, facial pain or visceral pain) or pain associated with cancer therapy (e.g. in response to chemotherapy, immunotherapy, hormonal therapy or radiotherapy). Back pain may be due to herniated or ruptured intervertabral discs or abnormalities of the lumber facet joints, sacroiliac joints, paraspinal muscles or the posterior longitudinal ligament. Back pain may resolve naturally but in some patients, where it lasts over 12 weeks, it becomes a chronic condition which can be particularly debilitating.

Nociceptive pain can also be related to inflammatory states. The inflammatory process is a complex series of biochemical and cellular events, activated in response to tissue injury or the presence of foreign substances, which results in swelling and pain (McMahon et al., 2006, Wall and Melzack's Textbook of Pain (5^{th} Ed), Chapter3). A common inflammatory condition associated with pain is arthritis. It has been estimated that almost 27 million Americans have symptomatic osteoarthritis (OA) or degenerative joint disease (Lawrence et al., 2008, Arthritis Rheum, 58, 15-35); most patients with osteoarthritis seek medical attention because of the associated pain. Arthritis has a significant impact on psychosocial and physical function and is known to be the leading cause of disability in later life. Rheumatoid arthritis is an immune-mediated, chronic, inflammatory polyarthritis disease, mainly affecting peripheral synovial joints. It is one of the commonest chronic inflammatory conditions in developed countries and is a major cause of pain.

In regard to nociceptive pain of visceral origin, visceral pain results from the activation of nociceptors of the thoracic, pelvic, or abdominal organs (Bielefeldt and Gebhart, 2006, Wall and Melzack's Textbook of Pain (5^{th}) Ed), Chapter48). This includes the reproductive organs, spleen, liver, gastrointestinal and urinary tracts, airway structures, cardiovascular system and other organs contained within the abdominal cavity. As such visceral pain refers to pain associated with conditions of such organs, such as painful bladder syndrome, interstitial cystitis, prostatitis, ulcerative colitis, Crohn's disease, renal colic, irritable bowl syndrome, endometriosis and dysmenorrheal (Classification of Chronic Pain, available at http://www.iasp-pain.org). Currently the potential for a neuropathic contribution (either through central changes or nerve injury/damage) to visceral pain states is poorly understood but may play a role in certain conditions (Aziz et al., 2009, Dig Dis 27, Suppl 1, 31-41)

Neuropathic pain is currently defined as pain arising as a direct consequence of a lesion or disease affecting the somatosensory system. Nerve damage can be caused by trauma and disease and thus the term ‘neuropathic pain’ encompasses many disorders with diverse aetiologies. These include, but are not limited to, peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy and vitamin deficiency. Neuropathic pain is pathological as it has no protective role. It is often present well after the original cause has dissipated, commonly lasting for years, significantly decreasing a patient's quality of life (Dworkin, 2009, Am J Med, 122, S1-S2; Geber et al., 2009, Am J Med, 122, S3-S12; Haanpaa et al., 2009, Am J Med, 122, S13-S21). The symptoms of neuropathic pain are difficult to treat, as they are often heterogeneous even between patients with the same disease (Dworkin, 2009, Am J Med, 122, S1-S2; Geber et al., 2009, Am J Med, 122, S3-S12; Haanpaa et al., 2009, Am J Med, 122, S13-S21). They include spontaneous pain, which can be continuous, and paroxysmal or abnormal evoked pain, such as hyperalgesia (increased sensitivity to a noxious stimulus) and allodynia (sensitivity to a normally innocuous stimulus).

It should be noted that some types of pain have multiple aetiologies and thus can be classified in more than one area, e.g. back pain, cancer pain and even migraine headaches may include both nociceptive and neuropathic components.

Similarly other types of chronic pain, perhaps less well understood, are not easily defined by the simplistic definitions of nociceptive or neuropathic. Such conditions include in particular fibromyalgia and chronic regional pain syndrome, which are often described as dysfunctional pain states e.g. fibromyalgia or complex regional pain syndrome (Woolf, 2010, J Clin Invest, 120, 3742-3744), but which are included in classifications of chronic pain states (Classification of Chronic Pain, available at http://www.iasp-pain.org).

As well as pain, and as noted in the background, trk inhibitors such as trkA inhibitors may be useful in treating a wide variety of other conditions and diseases.

### DETAILED DESCRIPTION

Embodiment 1 of the invention provides a compound of Formula I

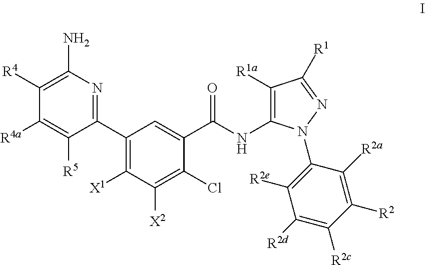

Or a prodrug thereof, or a pharmaceutically acceptable salt thereof, wherein

n is an integer from 0 to 4,
R¹ is CON(H or C₁₋₆ alkyl optionally substituted by 1 or 2 substituents independently selected from F, OH and OMe)(H or C₁₋₆ alkyl optionally substituted by 1, 2 or 3 substituents independently selected from F, NH₂, OH and OMe), CONR^{x1}(C₃₋₆cycloalkyl optionally substituted by 1, 2 or 3 substituents independently selected from OH, NH₂, CH₃ and CH₂OH), CONR^{x1}(CR^{Y}R^{X})ₘ(C₃₋₆cycloalkyl optionally substituted by 1, 2 or 3 substituents independently selected from OH, NH₂, CH₃ and CH₂OH), CONR^{x1}-Het, CO—NHet, CONR^{x1}(CR^{Y}R^{X})ₘ—CON(H or C₁₋₄ alkyl optionally substituted by 1 or 2 substituents independently selected from F, OH and OMe)(H or C₁₋₄ alkyl optionally substituted by 1, 2 or 3 substituents independently selected from F, OH and OMe), CONR^{x1}(CR^{Y}R^{X})ₘ—N(H or C₁₋₄ alkyl optionally substituted by 1 or 2 substituents independently selected from F, OH and OMe)(H or C₁₋₄ alkyl optionally substituted by 1, 2 or 3 substituents independently selected from F, OH and OMe), CONR^{x1}(CR^{Y}R^{X})ₘN(H or C₁₋₄ alkyl optionally substituted by 1 or 2 substituents independently selected from F, OH and OMe)CO(C₁₋₄ alkyl optionally substituted by 1, 2 or 3 substituents independently selected from F, OH and OMe), CONR^{x1}C(O)-Het, C(O)NR^{x1}(CR^{Y}R^{X})ₘ-Het, CONR^{x1}—Ar, C(O)—NRₓ₁-Het, CN, CO₂H, or CO₂(C₁₋₄ alkyl optionally substituted by 1 or 2 substituents independently selected from F, OH and OMe),
m is an integer from 1 to 3,
Ar is phenyl optionally substituted by 1, 2 or 3 groups independently selected from C₁₋₆ alkyl, halogen, CN, CF₃, CF₃O, C₁₋₆ alkoxy, C₁₋₆ alkoxy-O—C(O)—, CONH₂, C₁₋₆ alkylthio, hydroxy-C₁₋₆ alkyl, C₁₋₆ alkyl-SO₂—, CO₂H and C₁₋₃ alkoxy-C₁₋₃ alkyl-O—C(O)—,
Het is a 4-7-membered saturated or unsaturated heterocyclic ring having at least 1, and up to 3, hetero ring atoms independently selected from N, O and S, and which ring is optionally substituted by 1, 2 or 3 substituents independently selected from halogen, OH, ═O, CN, CONH₂, O(C₁₋₆ alkyl optionally substituted by one or more F), C(O)(C₁₋₆ alkyl optionally substituted by one or more F), C₁₋₆ alkyl optionally substituted by one or more F, C₁₋₆ alkyl substituted by CN, C₁₋₆ alkyl substituted by up to 3 OH, C₁₋₆ alkyl substituted by CO₂(C₁₋₄ alkyl), C₁₋₆ alkyl substituted by one or more C₁₋₃ alkoxy, S(O)ₚ(C₁₋₆ alkyl optionally substituted by one or more F), CO₂(C₁₋₆ alkyl), C₃₋₆ cycloalkyl, C(O)(C₃₋₆ cycloalkyl), N(H or C₁₋₃alkyl)CO(C₁₋₃ alkyl) and N(H or C₁₋₃ alkyl)(H or C₁₋₃ alkyl),
NHet is a 4-7-membered saturated or unsaturated heterocyclic ring with a ring N atom directly linked to the C(O) moiety, having from 0 to 2 further hetero ring atoms independently selected from N, O and S, and which ring is optionally substituted by up to 3 substituents independently selected from halogen, OH, ═O, CN, CONH₂, C₁₋₆ alkyl optionally substituted by one or more F, O(C₁₋₆ alkyl optionally substituted by one or more F), C(O)(C₁₋₆ alkyl optionally substituted by one or more F), C₁₋₆ alkyl substituted by CN, C₁₋₆ alkyl substituted by up to 3 OH, C₁₋₆ alkyl substituted by CO₂(C₁₋₄ alkyl), C₁₋₆ alkyl substituted by one or more C₁₋₃ alkoxy, S(O)ₚ(C₁₋₆ alkyl optionally substituted by one or more F), CO₂(C₁₋₆ alkyl), C₃₋₆ cycloalkyl, C(O)(C₃₋₆ cycloalkyl), N(H or C₁₋₃ alkyl)CO(C₁₋₃ alkyl), and N(H or C₁₋₃ alkyl)(H or C₁₋₃ alkyl),
R^{2a}, R², R^{2c}, R^{2d} and R^{2e} are each independently selected from H, OH, halogen, NH₂, or methyl optionally substituted by up to 3 F,
X¹, X², R^{1a}, R⁴, R^{4a} and R⁵ are each independently selected from H, C₃₋₆ cycloalkyl, C₀₋₆ alkyl optionally substituted by up to 3 substituents independently selected from halogen, CN, CO₂H, OH, (C₁₋₆ alkoxy optionally substituted by up to 3 F), S(O)ₚ(C₁₋₆ alkyl optionally substituted by up to 3 F), C(O)(C₁₋₆ alkoxy optionally substituted by up to 3 F or by C₁₋₃ alkoxy), C(O)NR^{x1}R^{x2}, NR^{x1}R^{x2}, O(C₃₋₆ cycloalkyl), Ar, Het, CO₂(C₁₋₆ alkyl optionally substituted by up to 3 F), NR^{x1}C(O)(C₁₋₆ alkyl optionally substituted by up to 3 F), NR^{x1}C(O)NR^{x2}(C₁₋₆ alkyl optionally substituted by up to 3 F), OC(O)(C₁₋₆ alkyl optionally substituted by up to 3 F), and OC(O)NR^{x1}R^{x2},
p is 0, 1 or 2,
R^{x1} and R^{x2} are each independently H, C₁₋₃ alkyl optionally substituted by up to 3 substituents independently selected from F, OH and OCH₃, or, together with the nitrogen atom to which they are attached, form a 4- to 6-membered saturated ring,
and R^{x} and R^{y} are each independently H, C₁₋₃ alkoxy optionally substituted by up to 3 F, C₁₋₃ alkyl optionally substituted by up to 3 substituents independently selected from F, OH and OCH₃, or, together with the carbon atom to which they are attached, are C₃₋₆ cycloalkyl.

Embodiment 2 is a compound, prodrug, or salt according to embodiment 1 wherein R^{1a} is H.

Embodiment 3 is a compound, prodrug, or salt according to embodiment 1 or 2 wherein R^{4a} is H.

Embodiment 4 is a compound, prodrug, or salt according to embodiment 1, 2 or 3 wherein R^{2a}, R², R^{2c}, R^{2d} and R^{2e} are each independently selected from H, F and OH.

Embodiment 5 is a compound, prodrug, or salt according to embodiment 1, 2, 3 or 4 wherein R^{2a}, R^{2c}, R^{2d} and R^{2e} are H.

Embodiment 6 is a compound, prodrug, or salt according to embodiment 1, 2, 3, 4 or 5 wherein R² is H or OH.

Embodiment 7 is a compound, prodrug, or salt according to embodiment 1, which is of Formula I′

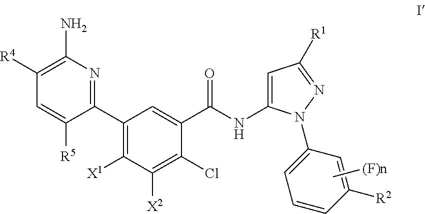

wherein
n is an integer from 0 to 4,
R¹ is CON(H or C₁₋₄ alkyl optionally substituted by 1 or 2 substituents independently selected from F, OH and OMe)(H or C₁₋₄ alkyl optionally substituted by 1, 2 or 3 substituents independently selected from F, NH₂, OH and OMe), CONH(C₃₋₆cycloalkyl optionally substituted by 1, 2 or 3 substituents independently selected from OH, NH₂, CH₃ and CH₂OH), CONH(CR^{Y}R^{X})ₘ(C₃₋₆cycloalkyl optionally substituted by 1, 2 or 3 substituents independently selected from OH, NH₂, CH₃ and CH₂OH), CONH-Het, CO—NHet, CONH(CR^{Y}R^{X})ₘ—CON(H or C₁₋₄ alkyl optionally substituted by 1 or 2 substituents independently selected from F, OH and OMe)(H or C₁₋₄ alkyl optionally substituted by 1, 2 or 3 substituents independently selected from F, OH and OMe), CONH(CR^{Y}R^{X})ₘ—N(H or C₁₋₄ alkyl optionally substituted by 1 or 2 substituents independently selected from F, OH and OMe)(H or C₁₋₄ alkyl optionally substituted by 1, 2 or 3 substituents independently selected from F, OH and OMe), CONH(CR^{Y}R^{X})ₘN(H or C₁₋₄ alkyl optionally substituted by 1 or 2 substituents independently selected from F, OH and OMe)CO(C₁₋₄ alkyl optionally substituted by 1, 2 or 3 substituents independently selected from F, OH and OMe), CONHC(O)-Het, C(O)NH(CR^{Y}R^{X})ₘ-Het, CONH—Ar, C(O)—NH-Het, CN, CO₂H, CO₂(C₁₋₄ alkyl optionally substituted by 1 or 2 substituents independently selected from F, OH and OMe),
m is an integer from 1 to 3,
Ar is phenyl optionally substituted by 1, 2 or 3 groups independently selected from C₁₋₆ alkyl, halogen, CN, CF₃, CF₃O, C₁₋₆ alkoxy, C₁₋₆ alkoxy-O—C(O)—, CONH₂, C₁₋₆ alkylthio, hydroxy-C₁₋₆ alkyl, C₁₋₆ alkyl-SO₂—, CO₂H and C₁₋₃ alkoxy-C₁₋₃ alkyl-O—C(O)—,
Het is a 4-7-membered saturated or unsaturated heterocyclic ring having at least 1, and up to 3, hetero ring atoms independently selected from N, O and S, and which ring is optionally substituted by 1, 2 or 3 substituents independently selected from halogen, OH, ═O, CN, CONH₂, O(C₁₋₆ alkyl optionally substituted by one or more F), C(O)(C₁₋₆ alkyl optionally substituted by one or more F), C₁₋₆ alkyl optionally substituted by one or more F, C₁₋₆ alkyl substituted by CN, C₁₋₆ alkyl substituted by up to 3 OH, C₁₋₆ alkyl substituted by CO₂(C₁₋₄ alkyl), C₁₋₆ alkyl substituted by one or more C₁₋₃ alkoxy, SO₂(C₁₋₆ alkyl optionally substituted by one or more F), CO₂(C₁₋₆ alkyl), O₃₋₆ cycloalkyl, C(O)(C₃₋₆ cycloalkyl), N(H or C₁₋₃ alkyl)CO(C₁₋₃ alkyl), N(H or C₁₋₃ alkyl)(H or C₁₋₃ alkyl),
NHet is a 4-7-membered saturated or unsaturated heterocyclic ring with a ring N atom directly linked to the C(O) moiety, having from 0 to 2 further hetero ring atoms independently selected from N, O and S, and which ring is optionally substituted by up to 3 substituents independently selected from halogen, OH, ═O, CN, CONH₂, C₁₋₆ alkyl optionally substituted by one or more F, O(C₁₋₆ alkyl optionally substituted by one or more F), C(O)(C₁₋₆ alkyl optionally substituted by one or more F), C₁₋₆ alkyl substituted by CN, C₁₋₆ alkyl substituted by up to 3 OH, C₁₋₆ alkyl substituted by CO₂(C₁₋₄ alkyl), C₁₋₆ alkyl substituted by one or more C₁₋₃ alkoxy, SO₂(C₁₋₆ alkyl optionally substituted by one or more F), CO₂(C₁₋₆ alkyl), C₃₋₆ cycloalkyl, C(O)(C₃₋₆ cycloalkyl), N(H or C₁₋₃ alkyl)CO(C₁₋₃ alkyl), N(H or C₁₋₃ alkyl)(H or C₁₋₃ alkyl),

### R² is H or OH,

X¹ is H, halogen, CN, CO₂H, C₁₋₆ alkyl optionally substituted by up to 3 F, C₁₋₆ alkyl optionally substituted by up to 3 OH, C₁₋₆ alkoxy optionally substituted by one or more F, C(═O)(C₁₋₆ alkoxy optionally substituted by up to 3 F, or by C₁₋₃ alkoxy), SO₂(C₁₋₆ alkyl optionally substituted by up to 3 F), S(C₁₋₆ alkyl optionally substituted by up to 3 F), (CH₂)₀₋₃(C(═O)NR^{x1}R^{x2}, or C₃₋₆ cycloalkyl,
X² is H, halogen, CN, CO₂H, C₁₋₆ alkyl optionally substituted by up to 3 F, C₁₋₆ alkyl optionally substituted by up to 3 OH, C₁₋₆ alkoxy optionally substituted by one or more F, C(═O)(C₁₋₆ alkoxy optionally substituted by up to 3 F, or by C₁₋₃ alkoxy), SO₂(C₁₋₆ alkyl optionally substituted by up to 3 F), S(C₁₋₆ alkyl optionally substituted by up to 3 F), (CH₂)₀₋₃(C(═O)NR^{x1}R^{x2}, or C₃₋₆ cycloalkyl,
R⁴ is H, halogen, CN, CO₂H, C₁₋₆ alkyl optionally substituted by up to 3 F, C₁₋₆ alkyl optionally substituted by up to 3 OH, C₁₋₆ alkoxy optionally substituted by one or more F, C(═O)(C₁₋₆ alkoxy optionally substituted by up to 3 F, or by C₁₋₃ alkoxy), SO₂(C₁₋₆ alkyl optionally substituted by up to 3 F), S(C₁₋₆ alkyl optionally substituted by up to 3 F), (CH₂)₀₋₃(C(═O)NR^{x1}R^{x2}, or C₃₋₆ cycloalkyl,
R⁵ is H, halogen, CN, CO₂H, C₁₋₆ alkyl optionally substituted by up to 3 F, C₁₋₆ alkyl optionally substituted by up to 3 OH, C₁₋₆ alkoxy optionally substituted by one or more F, C(═O)(C₁₋₆ alkoxy optionally substituted by up to 3 F, or by C₁₋₃ alkoxy), SO₂(C₁₋₆ alkyl optionally substituted by up to 3 F), S(C₁₋₆ alkyl optionally substituted by up to 3 F), (CH₂)₀₋₃(C(═O)NR^{x1}R^{x2}, or C₃₋₆ cycloalkyl,
R^{x1} and R^{x2} are each independently H or C₁₋₃ alkyl, or, together with the nitrogen atom to which they are attached, form a 4- to 6-membered saturated ring,
and R^{x} and R^{y} are each independently H or C₁₋₃ alkyl, or, together with the carbon atom to which they are attached, are C₃₋₆ cycloalkyl.

Embodiment 8 is a compound, prodrug, or salt according to embodiment 1, which is of Formula I″

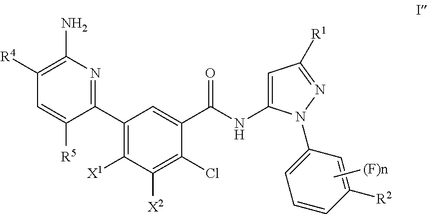

or a prodrug, or a pharmaceutically acceptable salt thereof, wherein
n is an integer from 0 to 4,
R¹ is CON(H or C₁₋₄ alkyl optionally substituted by 1 or 2 substituents independently selected from F, OH and OMe)(H or C₁₋₄ alkyl optionally substituted by 1, 2 or 3 substituents independently selected from F, NH₂, OH and OMe), CONH(C₃₋₆ cycloalkyl optionally substituted by 1, 2 or 3 substituents independently selected from OH, NH₂, CH₃ and CH₂OH), CONH(CR^{Y}R^{X})ₘ(C₃₋₆cycloalkyl optionally substituted by 1, 2 or 3 substituents independently selected from OH, NH₂, CH₃ and CH₂OH), CONH-Het, CONH(CR^{Y}R^{X})m-CON(H or C₁₋₄ alkyl optionally substituted by 1 or 2 substituents independently selected from F, OH and OMe)(H or C₁₋₄ alkyl optionally substituted by 1, 2 or 3 substituents independently selected from F, OH and OMe), CONH(CR^{Y}R^{X})ₘ—N(H or C₁₋₄ alkyl optionally substituted by 1 or 2 substituents independently selected from F, OH and OMe)(H or C₁₋₄ alkyl optionally substituted by 1, 2 or 3 substituents independently selected from F, OH and OMe), CONH(CR^{Y}R^{X})ₘN(H or C₁₋₄ alkyl optionally substituted by 1 or 2 substituents independently selected from F, OH and OMe)CO(C₁₋₄ alkyl optionally substituted by 1, 2 or 3 substituents independently selected from F, OH and OMe), CONHC(O)-Het, C(O)NH(CR^{Y}R^{X})ₘ-Het, CONH—Ar, C(O)—NH-Het, CN, CO₂H, and CO₂(C₁₋₄ alkyl optionally substituted by 1 or 2 substituents independently selected from F, OH and OMe),
m is an integer from 1 to 3,
Ar is phenyl optionally substituted by 1, 2 or 3 groups independently selected from C₁₋₆ alkyl, halogen, CN, CF₃, CF₃O, C₁₋₆ alkoxy, C₁₋₆ alkoxy-O—C(O)—, CONH₂, C₁₋₆ alkylthio, hydroxy-C₁₋₆ alkyl, C₁₋₆ alkyl-SO₂—, CO₂H and C₁₋₃ alkoxy-C₁₋₃ alkyl-O—C(O)—,
Het is a 4-7-membered saturated or unsaturated heterocyclic ring having at least 1, and up to 3, hetero ring atoms independently selected from N, O and S, and which ring is optionally substituted by 1, 2 or 3 substituents independently selected from halogen, OH, ═O, CN, CONH₂, O(C₁₋₆ alkyl optionally substituted by one or more F), C(O)(C₁₋₆ alkyl optionally substituted by one or more F), C₁₋₆ alkyl optionally substituted by one or more F, C₁₋₆ alkyl substituted by CN, C₁₋₆ alkyl substituted by up to 3 OH, C₁₋₆ alkyl optionally substituted by CO₂(C₁₋₄ alkyl), C₁₋₆ alkyl substituted by one or more C₁₋₃ alkoxy, SO₂(C₁₋₆ alkyl optionally substituted by one or more F), CO₂(C₁₋₆ alkyl), C₃₋₆ cycloalkyl, C(O)(C₃₋₆ cycloalkyl) and N(H or C₁₋₃ alkyl)CO(C₁₋₃ alkyl),
NHet is a 4-7-membered saturated or unsaturated heterocyclic ring with a ring N atom directly linked to the C(O) moiety to which it is attached, and having from 0 to 2 further hetero ring atoms independently selected from N, O and S, and which ring is optionally substituted by up to 3 substituents independently selected from halogen, OH, ═O, CN, CONH₂, C₁₋₆ alkyl optionally substituted by one or more F, O(C₁₋₆ alkyl optionally substituted by one or more F), C(O)(C₁₋₆ alkyl optionally substituted by one or more F), C₁₋₆ alkyl substituted by CN, C₁₋₆ alkyl substituted by up to 3 OH, C₁₋₆ alkyl optionally substituted by CO₂(C₁₋₄ alkyl), C₁₋₆ alkyl substituted by one or more C₁₋₃ alkoxy, SO₂(C₁₋₆ alkyl optionally substituted by one or more F), CO₂(C₁₋₆ alkyl), C₃₋₆ cycloalkyl, C(O)(C₃₋₆ cycloalkyl), and N(H or C₁₋₃ alkyl)CO(C₁₋₃ alkyl),

### R² is H or OH,

X¹ is H, Cl, F, CN, C₁₋₃ alkyl optionally substituted by one or more F, C₁₋₃ alkoxy optionally substituted by one or more F, or cyclopropyl,
X² is H, Cl, F, CN, C₁₋₃ alkyl optionally substituted by one or more F, C₁₋₃ alkoxy optionally substituted by one or more F, or cyclopropyl,
R⁴ is H, F, Cl, CN, C₁₋₃ alkyl optionally substituted by one or more F, C₁₋₃ alkoxy optionally substituted by one or more F, or cyclopropyl,
R⁵ is H, Cl, F, CN, C₁₋₃ alkyl optionally substituted by one or more F, C₁₋₃ alkoxy optionally substituted by one or more F, or cyclopropyl,
and R^{x} and R^{y} are each independently H or C₁₋₃ alkyl.

Embodiment 9 is a compound, prodrug, or salt according to any previous embodiment wherein R¹ is selected from CON(H or C₁₋₄ alkyl optionally substituted by 1 or 2 substituents independently selected from F, OH and OMe)(H or C₁₋₄ alkyl optionally substituted by 1, 2 or 3 substituents independently selected from F, NH₂, OH and OMe), CONH(C₃₋₆ cycloalkyl optionally substituted by 1, 2 or 3 substituents independently selected from OH, NH₂, CH₃ and CH₂OH), CONH-Het, and C(O)NH(CR^{Y}R^{X})ₘ-Het.

Embodiment 10 is a compound, prodrug, or salt according to any previous embodiment wherein R¹ is selected from CONH(H, CH₃ or C₂₋₄ alkyl optionally substituted by F, NH₂, OH or OMe), CONH(C₃₋₆ cycloalkyl optionally substituted by 1, 2 or 3 substituents independently selected from OH, CH₃ and CH₂OH), CONH-Het, and C(O)NH(CR^{Y}R^{X})ₘ-Het1, where Het1 is a 5- or 6-membered unsaturated heterocyclic ring having from 1 to 3 N ring atoms, and which ring is optionally substituted by up to 3 substituents independently selected from C₁₋₆ alkyl optionally substituted by one or more F.

Embodiment 11 is a compound, prodrug, or salt according to any previous embodiment wherein R¹ is selected from CONH(H, CH₃ or C₂₋₄ alkyl optionally substituted by OH), CONH(C₃₋₆ cycloalkyl optionally substituted by 1, 2 or 3 substituents independently selected from OH, CH₃ and CH₂OH), CONH-Het, and C(O)NH(CR^{Y}R^{X})ₘ-Het1,

where Het1 is a 5- or 6-membered unsaturated heterocyclic ring having from 1 to 3 N ring atoms, and which ring is optionally substituted by up to 3 substituents independently selected from C₁₋₆ alkyl optionally substituted by one or more F.

Embodiment 12 is a compound, prodrug, or salt according to any previous embodiment wherein R¹ is selected from CONH(pyrazolyl or 1,2,3-triazolyl optionally substituted by 1 or 2 methyl groups; 2-methyl-2-hydroxypropyl or 2-hydroxyethyl).

Embodiment 13 is a compound, prodrug, or salt according to any previous embodiment wherein R¹ is selected from

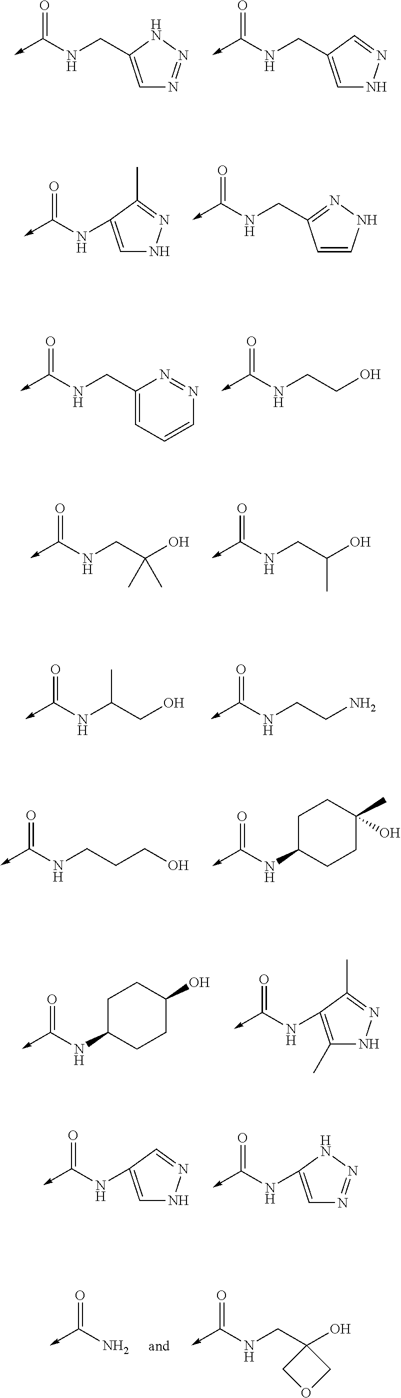

Embodiment 14 is a compound, prodrug, or salt according to embodiment 1 wherein R¹ is selected from the R¹ groups present in the compounds of the Examples herein.

Embodiment 15 is a compound, prodrug, or salt according to embodiment 1 wherein R¹, R^{1a}, R², R^{2a}, R^{2c}, R^{2d}, R^{2e}, X¹, X², R⁴, R^{4a}, and R⁵ are selected from the relevant groups in the compounds of the Examples herein.

Embodiment 16 is a compound, prodrug, or salt according to claim 1 wherein R¹ is selected from the R¹ groups present in the compounds of Examples 124, 101, 120, 13, 52, 77, 17, 117, 12, 41, 20, 24, 1, 19, 72, 135, 136, 9, 28, 127, 131, 22, 15, 116, 25, 16, and 82.

Embodiment 17 is a compound, prodrug, or salt according to any one of embodiments 1 to 16 wherein n is 0.

Embodiment 18 is a compound, prodrug, or salt according to any one of embodiments 1 to 118 wherein X¹ is H, F or Cl.

Embodiment 19 is a compound, prodrug, or salt according to any one of embodiments 1 to 18 wherein X² is H.

Embodiment 20 is a compound, prodrug, or salt according to any one of embodiments 1 to 19 wherein R⁴ is H, F, Cl, CH₃, CN or OCH₃.

Embodiment 21 is a compound, prodrug, or salt according to any one of embodiments 1 to 20 wherein R⁵ is H, F, Cl, CH₃, CN or OCH₃.

Embodiment 22 is a compound of formula I′″

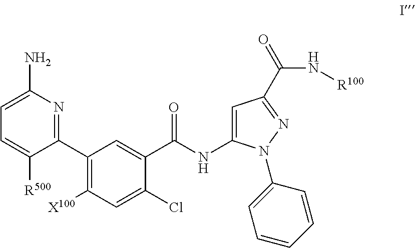

### Wherein

### R⁵⁰⁰ is H or F,

### X¹⁰⁰ is H, For CI,

And R¹⁰⁰ is pyrazol-4-yl optionally substituted by 1 or 2 methyl groups, 1,2,3-triazol-4-yl optionally substituted by methyl, 2-hydroxyethyl, or 2-methyl-2-hydroxypropyl, or prodrug, or pharmaceutically acceptable salt thereof.

Embodiment 23 is a compound selected form any of the Examples herein, or a prodrug or pharmaceutically acceptable salt thereof.

Embodiment 24 is a compound selected from the compounds of Example 124, 101, 120, 13, 52, 77, 17, 117, 12, 41, 20, 24, 1, 19, 72, 135, 9, 28, 127, 131, 22, 15, 116, 25, 16, and 82, or a prodrug, or a pharmaceutically acceptable salt thereof.

Embodiment 25 is a prodrug according to any previous embodiment wherein the prodrug moiety is a phosphate ester.

Embodiment 26 is a pharmaceutical composition comprising a compound of the formula I or a prodrug, or a pharmaceutically acceptable salt thereof, as defined in any one of the preceding embodiments 1 to 25, and a pharmaceutically acceptable carrier.

Embodiment 26 is a compound of the formula I or a prodrug, or a pharmaceutically acceptable salt thereof, as defined in any one of embodiments 1 to 25, for use as a medicament.

Embodiment 27 is a compound of formula I or a prodrug, or a pharmaceutically acceptable salt thereof, as defined in any one of embodiments 1 to 25 for use in the treatment of a disease for which an TrkA receptor antagonist is indicated.

Embodiment 28 is a compound of formula I or a prodrug, or a pharmaceutically acceptable salt thereof, as defined in any one of embodiments 1 to 25 for use in the treatment of pain or cancer.

Embodiment 29 is the use of a compound of the formula I or a prodrug, or a pharmaceutically acceptable salt or composition thereof, as defined in any one of embodiments 1 to 25, for the manufacture of a medicament to treat a disease for which a TrkA receptor antagonist is indicated.

Embodiment 30 is the use of a compound of the formula I or a prodrug, or a pharmaceutically acceptable salt or composition thereof, as defined in any one of embodiments 1 to 25, for the manufacture of a medicament to treat pain or cancer.

Embodiment 31 is a method of treatment of a mammal, to treat a disease for which a TrkA receptor antagonist is indicated, comprising treating said mammal with an effective amount of a compound of the formula I or a prodrug, or a pharmaceutically acceptable salt thereof, as defined in any one of embodiments 1 to 25.

Embodiment 32 is a method of treatment of pain or cancer in a mammal, comprising treating said mammal with an effective amount of a compound of the formula I or a prodrug, or a pharmaceutically acceptable salt thereof, as defined in any one of embodiments 1 to 25.

Embodiment 33 is a compound or a prodrug, or salt according to any one of embodiments 1 to 25 for use in a medical treatment in combination with a further drug substance.

Further embodiments include:

Any novel genus of intermediates described in the Schemes below;
Any novel specific intermediate described in the Preparations below;
Any novel process described herein.

“Halogen” means a fluoro, chloro, bromo or iodo group.

“Alkyl” groups, containing the requisite number of carbon atoms, can be unbranched or branched. Examples of alkyl include methyl, ethyl, n-propyl, i-propyl, n-butyl, i-butyl, sec-butyl and t-butyl.

“Het is a 4-7-membered saturated or unsaturated heterocyclic ring” includes fully saturated, partially unsaturated and fully unsaturated rings. Typical, but non-limiting, ring moieties include the following: oxetanyl, thiatanyl, azetidinyl, tetrahydrofuranyl, tetrahydrothiophenyl, pyrrolidinyl, tetrahydropyranyl, tetrahydrothiopyranyl, piperidinyl, 1,4-dioxanyl, 1,4-oxathianyl, morpholinyl, 1,4-dithianyl, piperazinyl, 1,4-azathianyl, oxepanyl, thiepanyl, azepanyl, 1,4-dioxepanyl, 1,4-oxathiepanyl, 1,4-oxaazepanyl, 1,4-dithiepanyl, 1,4-thieazapanyl, 1,4-diazepinyl, 3,4-dihydro-2H-pyranyl, 5,6-dihydro-2H-pyranyl, 2H-pyranyl, 1,2,3,4-tetrahydropyridinyl, 1,2,5,6-tetrahydropyridinyl, pyrrolyl, furanyl, thiophenyl, pyrazolyl, imidazolyl, isoxazolyl, oxazolyl, isothiazolyl, thiazolyl, 1,2,3-trazolyl, 1,3,4-trazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, pyridinyl, pyridazinyl, pyrimidinyl, pyrazinyl, etc.

“Substituted by” means that substituents are present only if valency allows.

“Pharmaceutically acceptable salts” of the compounds of formula I include the acid addition and base addition salts (including disalts, hemisalts, etc.) thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base addition salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

For a review on suitable salts, see “Handbook of Pharmaceutical Salts: Properties, Selection, and Use” by Stahl and Wermuth (Wiley-VCH, Weinheim, Germany, 2002).

The compounds of the invention may be administered as prodrugs. Thus certain derivatives of compounds of formula (I) which may have little or no pharmacological activity themselves can, when administered into or onto the body, be converted into compounds of formula (I) having the desired activity, for example, by hydrolytic or enzymatic cleavage. Such derivatives are referred to as ‘prodrugs’. Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association).

Prodrugs can, for example, be produced by replacing appropriate functionalities present in a compound of formula (I) with certain moieties known to those skilled in the art as ‘pro-moieties’ as described, for example, in “Design of Prodrugs” by H Bundgaard (Elsevier, 1985).

Examples of prodrugs include phosphate prodrugs, such as dihydrogen or dialkyl (e.g. di-tert-butyl) phosphate prodrugs.

Further examples of replacement groups in accordance with the foregoing examples and examples of other prodrug types may be found in the aforementioned references. Specific prodrug groups envisaged for, and included in the definition of, the invention include: phosphate esters of alcohols “ROH”, e.g. RO—P(═O)(OH2)2 or salts thereof; and amino acid esters of alcohols “ROH”, e.g. RO—C(═O)—C*—NH₂ wherein NH2-C*—CO2H is an amino acid such as histidine, alanine, isoleucine, leucine, lysine, methionine, phenylalanine, threonine, tryptophan, valine, arginine, asparagine, aspartic acid, cysteine, glutamic acid, glutamine, glycine, ornithine, proline, selenocysteine, tyrosine;

Or derivative thereof such as dimethylglycine, and the like.

The compounds of the invention include compounds of formula I, prodrugs and salts thereof as hereinbefore defined, polymorphs, and isomers thereof (including optical, geometric and tautomeric isomers) as hereinafter defined and isotopically-labelled compounds of formula I.

Unless otherwise specified, compounds of formula (I) containing one or more asymmetric carbon atoms can exist as two or more stereoisomers. Where a compound of formula (I) contains for example, a keto or guanidine group or an aromatic moiety, tautomeric isomerism (‘tautomerism’) can occur. It follows that a single compound may exhibit more than one type of isomerism.

Included within the scope of the claimed compounds of the present invention are all stereoisomers, geometric isomers and tautomeric forms of the compounds of formula (I), including compounds exhibiting more than one type of isomerism, and mixtures of one or more thereof. Also included are acid addition or base addition salts wherein the counterion is optically active, for example, D-lactate or L-lysine, or racemic, for example, DL-tartrate or DL-arginine.

Examples of types of potential tautomerisms shown by the compounds of the invention include hydroxypyridine [symbol] pyridone; amide [symbol] hydroxyl-imine and keto [symbol] enol tautomerisms:

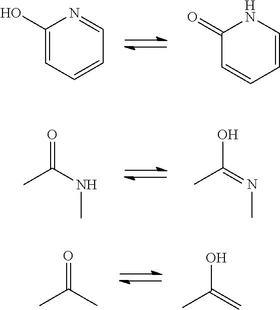

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or other derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on a resin with an asymmetric stationary phase and with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Mixtures of stereoisomers may be separated by conventional techniques known to those skilled in the art. [see, for example, “Stereochemistry of Organic Compounds” by E L Eliel (Wiley, New York, 1994).]

The present invention includes all pharmaceutically acceptable isotopically-labelled compounds of formula (I) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labelled compounds of formula (I) can generally be prepared by conventional techniques known to those skilled in the art or by processes analogous to those described in the accompanying Examples and Preparations using an appropriate isotopically-labelled reagents in place of the non-labelled reagent previously employed.

The routes below, including those mentioned in the Examples and Preparations, illustrate methods of synthesising compounds of formula I. The skilled person will appreciate that the compounds of the invention, and intermediates thereto, could be made by methods other than those specifically described herein, for example by adaptation of the methods described herein, for example by methods known in the art. Suitable guides to synthesis, functional group interconversions, use of protecting groups, etc., are for example: “Comprehensive Organic Transformations” by R C Larock, VCH Publishers Inc. (1989); Advanced Organic Chemistry” by J. March, Wiley Interscience (1985); “Designing Organic Synthesis” by S Warren, Wiley Interscience (1978); “Organic Synthesis—The Disconnection Approach” by S Warren, Wiley Interscience (1982); “Guidebook to Organic Synthesis” by R K Mackie and D M Smith, Longman (1982); “Protective Groups in Organic Synthesis” by T W Greene and P G M Wuts, John Wiley and Sons, Inc. (1999); and “Protecting Groups” by P J, Kocienski, Georg Thieme Verlag (1994); and any updated versions of said standard works.

In addition, the skilled person will appreciate that it may be necessary or desirable at any stage in the synthesis of compounds of the invention to protect one or more sensitive groups, so as to prevent undesirable side reactions. In particular, it may be necessary or desirable to protect amino or carboxylic acid groups. The protecting groups used in the preparation of the compounds of the invention may be used in conventional manner. See, for example, those described in ‘Greene's Protective Groups in Organic Synthesis’ by Theodora W Greene and Peter G M Wuts, third edition, (John Wiley and Sons, 1999), in particular chapters 7 (“Protection for the Amino Group”) and 5 (“Protection for the Carboxyl Group”), incorporated herein by reference, which also describes methods for the removal of such groups.

In the general synthetic methods below, unless otherwise specified, the substituents are as defined above with reference to the compounds of formula (I) above.

Where ratios of solvents are given, the ratios are by volume.

The compounds of the invention may be prepared by any method known in the art for the preparation of compounds of analogous structure. In particular, the compounds of the invention can be prepared by the procedures described by reference to the Schemes that follow, or by the specific methods described in the Examples, or by similar processes to either.

The skilled person will appreciate that the experimental conditions set forth in the schemes that follow are illustrative of suitable conditions for effecting the transformations shown, and that it may be necessary or desirable to vary the precise conditions employed for the preparation of compounds of formula (I). It will be further appreciated that it may be necessary or desirable to carry out the transformations in a different order from that described in the schemes, or to modify one or more of the transformations, to provide the desired compound of the invention.

All of the derivatives of formula (I) can be prepared by the procedures described in the general methods presented below or by routine modifications thereof. The present invention also encompasses any one or more of these processes for preparing the derivatives of formula (I), in addition to any novel intermediates used therein. The person skilled in the art will appreciate that the following reactions may be heated thermally or under microwave irradiation.

It will be further appreciated that it may be necessary or desirable to carry out the transformations in a different order from that described in the schemes, or to modify one or more of the transformations, to provide the desired compound of the invention.

According to a first process, compounds of formula I wherein (IA; R¹ is an amide), (IB; R¹ is CO₂H) and (IC; R¹ is an ester) may be prepared from compounds of formulae (VI) and (IV), as illustrated by Scheme 1,

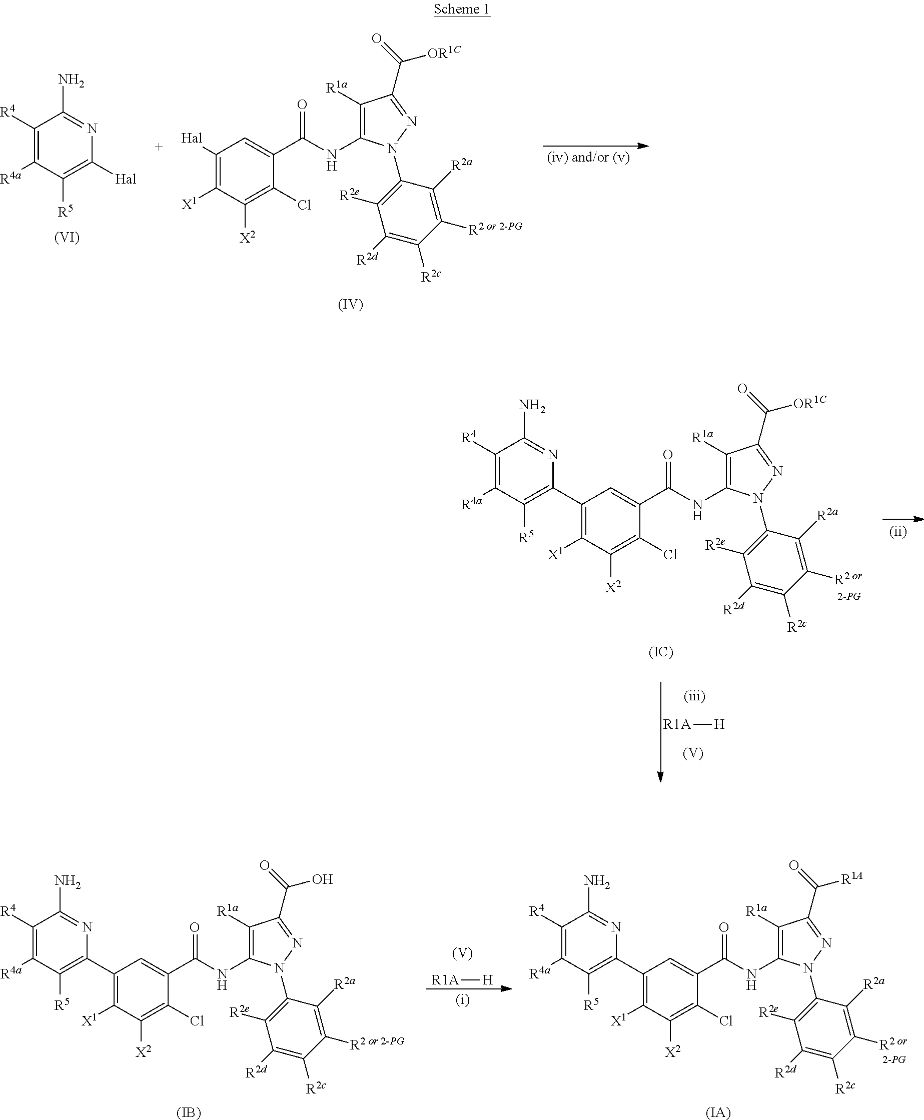

wherein CO₂R^{1C} corresponds to the ester groups of R¹ as defined herein, or a protected version thereof; COR^{1A} corresponds to the amide groups of R¹ as defined herein, or a protected version thereof; R^{2-PG} represents a protected hydroxyl group such as benzyloxy; Hal is chloro, bromo or iodo.

Compounds of formulae (V) and (VI) are commercially available or may be synthesized by those skilled in the art according to the literature or preparations described herein. Wherein compounds of formulae (IA) contain a protecting group such as tert-butyldimethylsilyl, a deprotection step may be employed—preferred conditions comprise TBAF in THF at room temperature. Wherein compounds of formulae (IA), (IB) or (IC) include a protecting group such as tert-butoxycarbonyl or tert-butyl, an acid mediated deprotection step may be employed at any stage in Scheme 1. Preferred conditions comprise aqueous HCl or TFA in dioxane or DCM at room temperature. Alternatively the protecting group may be removed in situ during steps (i)-(iv).

Wherein compounds of formula (IA), (IB) or (IC) include R^{2-PG} such as benzyloxy, deprotection may be employed following process step (i). Preferred conditions comprise boron tribromide in DCM at room temperature.

Compounds of formula (IA) may be prepared from compounds of formula (IB) according to process step (i), an amide bond formation step with compounds of formula (V) mediated by a suitable combination of amide bond coupling agent and organic base. Preferred conditions comprise HATU or HBTU with triethylamine or DIPEA, or EDCI with HOBt and triethylamine or DIPEA in either DCM or DMF at room or elevated temperatures of 80° C., or using 2,4,6-tripropyl-1,3,5,2,4,6-trioxatriphosphinane 2,4,6-trioxide in THF or 2-methyl-THF with pyridine or DIPEA at 85° C.

Alternatively compounds of formula (IA) may be prepared directly from compounds of formula (IC) according to process step (iii), a nucleophilic displacement reaction of an ester with amines of formula (V). Preferred conditions comprise heating compounds of formula (V) with compounds of formula (IC) either neat or in a solution of methanol at elevated temperatures of 60-80° C.

Compounds of formula (IB) may be prepared from compounds of formula (IC) according to process step (ii), a hydrolysis step mediated by an inorganic base. Preferred conditions comprise lithium hydroxide in methanol or ethanol at room temperature. Compounds of formula (IC) may be prepared from compounds of formula (IV) and (VI) according to process steps (iv) and/or (v), a Suzuki cross-coupling reaction preceeded if necessary by a boronic ester formation reaction. Typical Suzuki cross-coupling conditions comprise a palladium catalyst containing suitable phosphine ligands, in the presence of an inorganic base, in aqueous dioxane, at elevated temperatures either thermally or under microwave irradiation. Preferred conditions comprise Pd(dppf)Cl₂ or Pd(PPh₃)₄ with either sodium, cesium or potassium carbonate or cesium fluoride in aqueous dioxane or methanol at from room temperature to 120° C. Typical boronic ester formation conditions comprise Pd(dppf)Cl₂ and potassium acetate with bispinacolatodiboron with compounds of formula (IV) or (VI) in dioxane at reflux.

According to a second process, compounds of formula (IA) may be prepared in an alternative sequence from compounds of formulae (IV) as illustrated by Scheme 2.

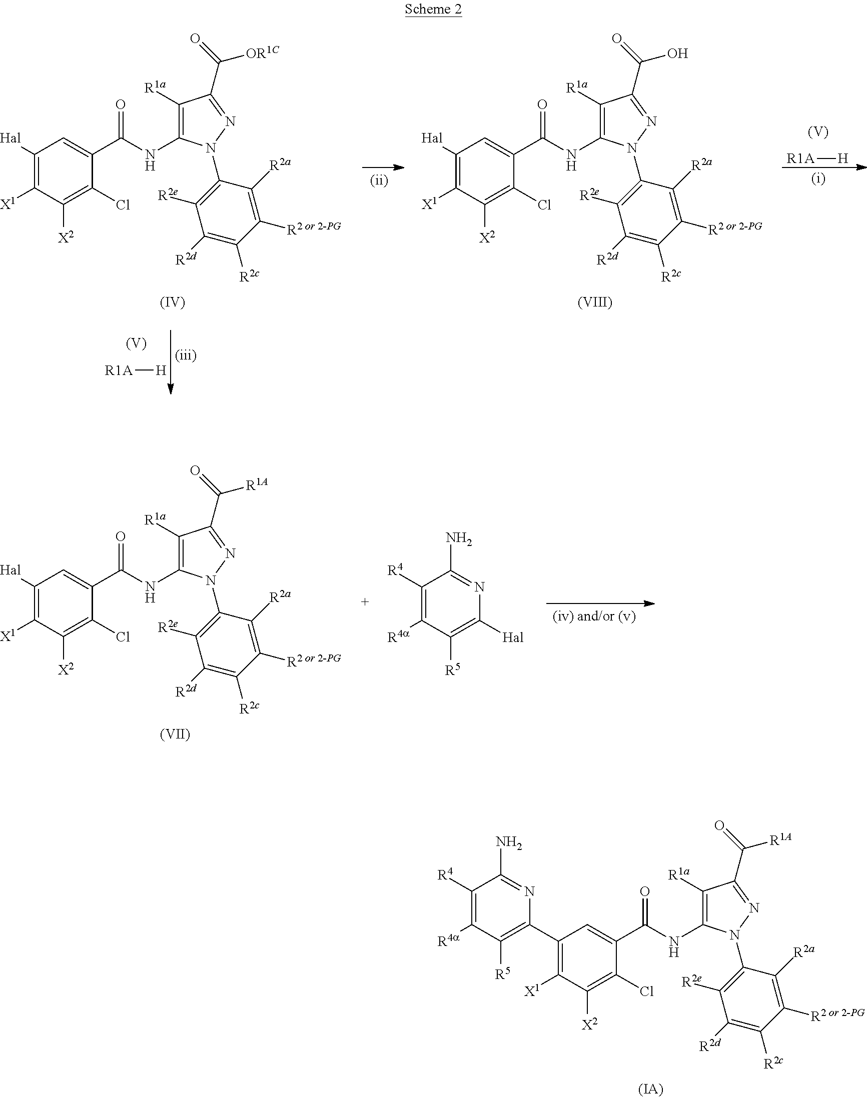

Wherein compounds of formula (IA) include a protecting group such as tert-butyldimethylsilyl, benzyl, tert-butoxycarbonyl or tert-butyl, deprotection may occur as necessary as described in Scheme 1.

Compounds of formula (IA) may be prepared from compounds of formula (VII) and (VI) according to process steps (iv) and/or (v) as described in Scheme 1.

Compounds of formula (VII) may be prepared from compounds of formulae (VIII) and (V) according to process step (i) as described in Scheme 1.

Compounds of formula (VIII) may be prepared from compounds of formula (IV) according to process step (ii) a hydrolysis step as described in Scheme 1.

Alternatively compounds of formula (VII) may be prepared from compound of formula (IV) according to process step (iii) a nucleophilic displacement reaction of an ester with amines of formula (V) as described in Scheme 1.

According to a third process, compounds of formula (ID; R¹ is CN) and (IE; R¹ is CONH₂) may be prepared from compounds of formulae (VI) and (X) as illustrated by Scheme 3,

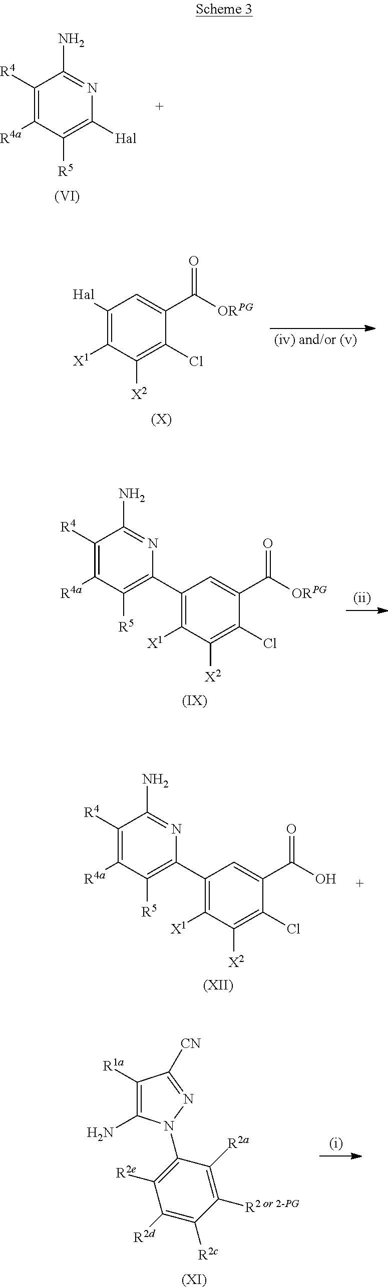

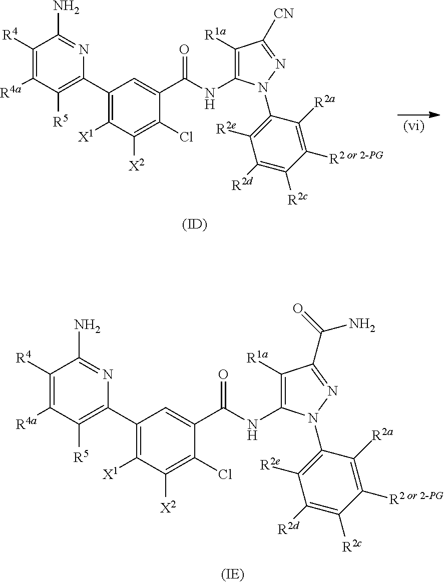

wherein R^{PG} is methyl or ethyl; Hal is chloro, bromo or iodo.

Compounds of formulae (X), (VI) and (XI) are commercially available or may be synthesized by those skilled in the art according to the literature or preparations described herein.

Introduction of a tert-butyldicarbonate protecting group may occur as necessary during Scheme 3, typically using di-tertbutyldicarbonate and triethylamine in DCM with catalytic DMAP at room temperature.

Wherein compounds of formulae (IX), (XII), (ID) and (IE) include a protecting group such as, benzyl, tert-butoxycarbonyl or tert-butyl, deprotection may occur as necessary as described in Scheme 1.

Compounds of formula (IE) may be prepared from compounds of formula (ID) according to process step (vi), an oxidative hydrolysis reaction. Preferred conditions comprise aqueous hydrogen peroxide with potassium carbonate in DMSO at room temperature. Compounds of formula (ID) may be prepared from compounds of formula (XI) and (XII) according to process step (i), an amide bond formation reaction as described in Scheme 1.

Compounds of formula (XII) may be prepared from compounds of formula (IX) according to process step (ii) as described in Scheme 1.

Compounds of formula (IX) may be prepared from compounds of formulae (X) and (VI) according to process steps (iv) and/or (v) as described in Scheme 1.

Compounds of formula (IX) may be further halogenated, for example by reaction with NCS in MeCN at 75° C.

According to a fourth process, compounds of formula (IE) may be prepared in an alternative sequence from compounds of formulae (XI) and (XIII) as illustrated by Scheme 4,

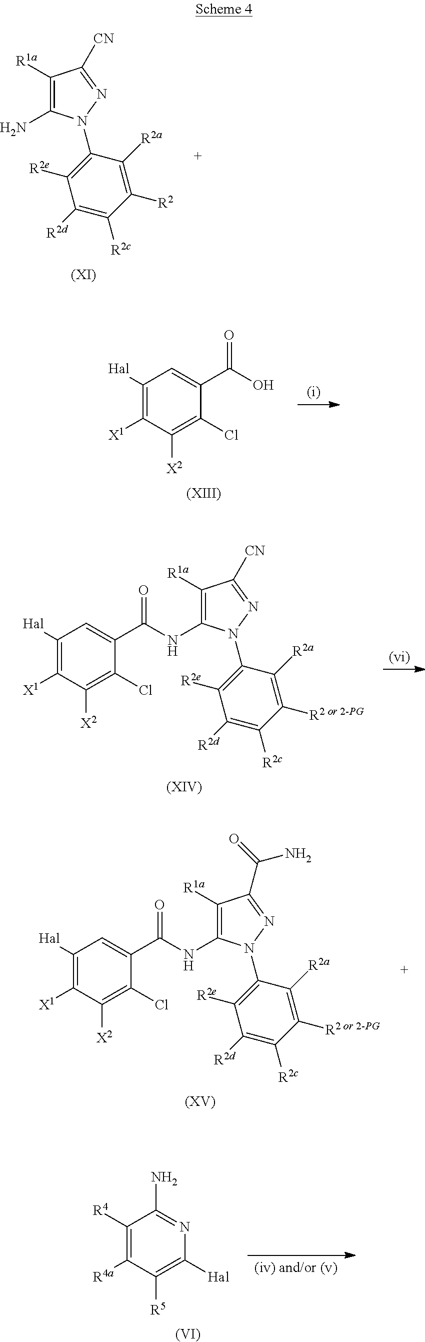

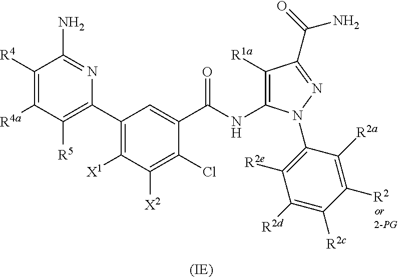

wherein Hal is chloro, bromo or iodo.

Compounds of formulae (XI), (XIII) and (VI) are commercially available or may be synthesized by those skilled in the art according to the literature or preparations described herein.

Wherein compounds of formulae (IE) include a protecting group such as benzyl, tert-butoxycarbonyl or tert-butyl, deprotection may occur as necessary as described in Scheme 1.

Compounds of formula (IE) may be prepared from compounds of formula (XV) and (VI) according to process steps (iv) and (v) as described in Scheme 1.

Compounds of formula (XV) may be prepared from compounds of formula (XIV) according to process step (vi) as described in Scheme 3.

Compounds of formula (XIV) may be prepared from compounds of formulae (XI) and (XIII) according to process step (i) as described in Scheme 1.

According to a fifth process, compounds of formula (IV) may be prepared from compounds of formulae (XIII) and (XVI) as illustrated by Scheme 5,

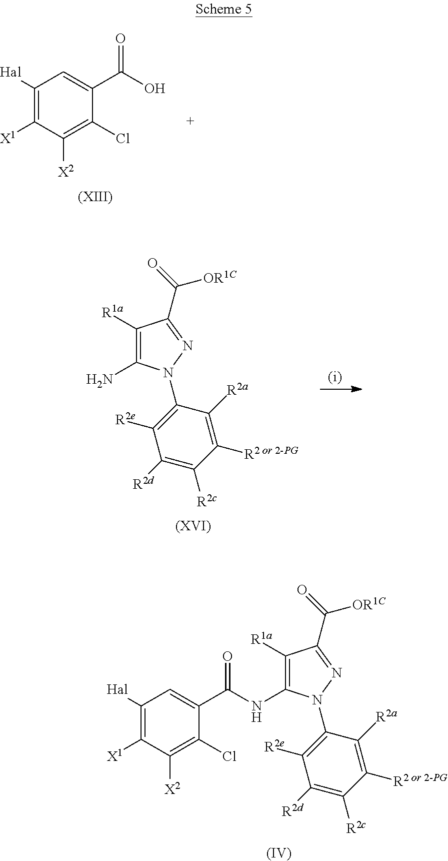

wherein Hal is chloro, bromo, iodo; R^{PG} is methyl or ethyl.

Compounds of formula (XIII) and (XVI) are commercially available or may be synthesized by those skilled in the art according to the literature or preparations described herein.

Compounds of formula (IV) may be prepared from compounds of formulae (XIII) and (XVI) according to process step (i) as described in Scheme 1.

According to a further embodiment the present invention provides novel intermediate compounds.

Pharmaceutically acceptable salts of a compound of formula (I) may be readily prepared by mixing together solutions of the compound of formula (I) and the desired acid or base, as appropriate. The salt may precipitate from solution and be collected by filtration or may be recovered by evaporation of the solvent. The degree of ionisation in the salt may vary from completely ionised to almost non-ionised.

The compounds of the invention intended for pharmaceutical use may be administered alone or in combination with one or more other compounds of the invention or in combination with one or more other drug agent (or as any combination thereof). Generally, they will be administered as a formulation in association with one or more pharmaceutically acceptable excipients. The term “excipient” is used herein to describe any biologically inactive ingredient other than the compounds and salts of the invention. The choice of excipient will to a large extent depend on factors such as the particular mode of administration, the effect of the excipient on solubility and stability, and the nature of the dosage form. For example, a compound of the formula I, or a pharmaceutically acceptable salt or solvate thereof, as defined above, may be administered simultaneously (e.g. as a fixed dose combination), sequentially or separately in combination with one or more other drug agent.

A compound of formula I may be usefully combined with another pharmacologically active compound, or with two or more other pharmacologically active compounds, particularly in the treatment of pain. The skilled person will appreciate that such combinations offer the possibility of significant advantages, including patient compliance, ease of dosing and synergistic activity.

In the combinations that follow the compound of the invention may be administered simultaneously, sequentially or separately in combination with the other therapeutic agent or agents.

A compound of formula (I), or a pharmaceutically acceptable salt thereof, as defined above, may be administered in combination with one or more agents selected from:
a selective Nav1.3 channel modulator, such as a compound disclosed in WO2008/118758;
a selective Nav1.7 channel modulator, such as a compound disclosed in WO2010/079443, e.g. 4-[2-(5-amino-1H-pyrazol-4-yl)-4-chlorophenoxy]-5-chloro-2-fluoro-N-1,3-thiazol-4-ylbenzenesulfonamide or 4-[2-(3-amino-1H-pyrazol-4-yl)-4-(trifluoromethyl)phenoxy]-5-chloro-2-fluoro-N-1,3-thiazol-4-ylbenzenesulfonamide, or a pharmaceutically acceptable salt of either;
a selective Nav1.8 channel modulator;
a selective Nav1.9 channel modulator;
a compound which modulates activity at more than one Nav channel, including a non-selective modulator such as bupivacaine, carbamazepine, lamotrigine, lidocaine, mexiletine or phenytoin;
any inhibitor of nerve growth factor (NGF) signaling, such as: an agent that binds to NGF and inhibits NGF biological activity and/or downstream pathway(s) mediated by NGF signaling (e.g. tanezumab), a TrkA antagonist or a p75 antagoinsist, or an agent that inhibits downstream signaling in regard to NGF stimulated TrkA or P75 signalling;
an inhibitor of neurotrophic pathways, where such inhibition is achieved by: (a) an agent that binds to nerve growth factor (NGF) (e.g. tanezumab, fasinumab or fulranumab), brain-derived neurotrophic factor (BDNF), neurotrophin-3 (NT-3) or neurotrophin-4 (NT-4), or to more than one of the aforementioned neurotrophins (e.g. soluble P75); or (b) an agent that inhibits receptor function at one or more of TrKA, TrKB, TrKC or P75, either at the orthosteric site, an allosteric site or by inhibition of the catalytic activity of the receptor(s);
a compound which increases the levels of endocannabinoid, such as a compound with fatty acid amid hydrolase inhibitory (FAAH) or monoacylglycerol lipase (MAGL) activity;
an analgesic, in particular paracetamol;
an opioid analgesic, such as: buprenorphine, butorphanol, cocaine, codeine, dihydrocodeine, fentanyl, heroin, hydrocodone, hydromorphone, levallorphan levorphanol, meperidine, methadone, morphine, nalmefene, nalorphine, naloxone, naltrexone, nalbuphine, oxycodone, oxymorphone, propoxyphene or pentazocine;
an opioid analgesic which preferentially stimulates a specific intracellular pathway, for example G-protein as opposed to beta arrestin recruitment, such as TRV130; an opioid analgesic with additional pharmacology, such as: noradrenaline (norepinephrine) reuptake inhibitory (NRI) activity, e.g. tapentadol; serotonin and norepinephrine reuptake inhibitory (SNRI) activity, e.g. tramadol; or nociceptin receptor (NOP) agonist activity, such as GRT6005;
a nonsteroidal antiinflammatory drug (NSAID), such as a non-selective cyclooxygenase (COX) inhibitor, e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin or zomepirac; or a COX-2 selective inhibitor, e.g. celecoxib, deracoxib, etoricoxib, mavacoxib or parecoxib;
a prostaglandin E₂ subtype 4 (EP4) antagonist;
a microsomal prostaglandin E synthase type 1 (mPGES-1) inhibitor;
a sedative, such as glutethimide, meprobamate, methaqualone or dichloralphenazone;
a GABA_{A} modulator with broad subtype modulatory effects mediated via the benzodiazepine binding site, such as chlordiazepoxide, alprazolam, diazepam, lorazepam, oxazepam, temazepam, triazolam, clonazepam or clobazam;
a GABA_{A} modulator with subtype-selective modulatory effects mediated via the benzodiazepine binding site with reduced adverse effects, for example sedation, such as TPA023, TPA023B, L-838,417, CTP354 or NSD72;
a GABA_{A} modulator acting via alternative binding sites on the receptor, such as barbiturates, e.g. amobarbital, aprobarbital, butabital, mephobarbital, methohexital, pentobarbital, phenobartital, secobarbital, or thiopental; neurosteroids such as alphaxalone, alphadolone or ganaxolone; β-subunit ligands, such as etifoxine; or δ-preferring ligands, such as gaboxadol;
a GlyR3 agonist or positive allosteric modulator;
a skeletal muscle relaxant, e.g. baclofen, carisoprodol, chlorzoxazone, cyclobenzaprine, metaxolone, methocarbamol or orphrenadine;
a glutamate receptor antagonist or negative allosteric modulator, such as an NMDA receptor antagonist, e.g. dextromethorphan, dextrorphan, ketamine or, memantine; or an mGluR antagonist or modulator;
an alpha-adrenergic, such as clonidine, guanfacine or dexmetatomidine;
a beta-adrenergic such as propranolol;
a tricyclic antidepressant, e.g. desipramine, imipramine, amitriptyline or nortriptyline;
a tachykinin (NK) antagonist, such as aprepitant or maropitant; a muscarinic antagonist, e.g oxybutynin, tolterodine, propiverine, tropsium chloride, darifenacin, solifenacin, temiverine and ipratropium;
a Transient Receptor Potential V1 (TRPV1) receptor agonist (e.g. resinferatoxin or capsaicin) or antagonist (e.g. capsazepine or mavatrap);
a Transient Receptor Potential A1 (TRPA1) receptor agonist (e.g. cinnamaldehyde or mustard oil) or antagonist (e.g. GRC17536 or CB-625);
a Transient Receptor Potential M8 (TRPM8) receptor agonist (e.g. menthol or icilin) or antagonist;
a Transient Receptor Potential V3 (TRPV3) receptor agonist or antagonist (e.g. GRC-15300);
a corticosteroid such as dexamethasone;
a 5-HT receptor agonist or antagonist, particularly a 5-HT_{1B/1D} agonist, such as eletriptan, sumatriptan, naratriptan, zolmitriptan or rizatriptan;
a 5-HT_{2A} receptor antagonist;
a cholinergic (nicotinic) analgesic, such as ispronicline (TC-1734), vareniclineor nicotine;
a PDEV inhibitor, such sildenafil, tadalafilor vardenafil;
an alpha-2-delta ligand such as gabapentin, gabapentin enacarbil or pregabalin;
a serotonin reuptake inhibitor (SRI) such as sertraline, demethylsertraline, fluoxetine, norfluoxetine, fluvoxamine, paroxetine, citalopram, desmethylcitalopram, escitalopram, d,l-fenfluramine, femoxetine, ifoxetine, cyanodothiepin, litoxetine, dapoxetine, nefazodone, cericlamine and trazodone;
anNRI, such as maprotiline, lofepramine, mirtazepine, oxaprotiline, fezolamine, tomoxetine, mianserin, buproprion, buproprion metabolite hydroxybuproprion, nomifensine and viloxazine, especially a selective noradrenaline reuptake inhibitor such as reboxetine;
an SNRI, such as venlafaxine, O-desmethylvenlafaxine, clomipramine, desmethylclomipramine, duloxetine, milnacipran and imipramine;
an inducible nitric oxide synthase (iNOS) inhibitor;
a leukotriene B4 antagonist;
a 5-lipoxygenase inhibitor, such as zileuton;
a potassium channel opener or positive modulator, such as an opener or positive modulator of KCNQ/Kv7 (e.g. retigabine or flupirtine), a G protein-coupled inwardly-rectifying potassium channel (GIRK), a calcium-activated potassium channel (Kca) or a potassium voltage-gated channel such as a member of subfamily A (e.g. Kv1.1), subfamily B (e.g. Kv2.2) or subfamily K (e.g. TASK, TREK or TRESK);
a P2X₃ receptor antagonist (e.g. AF219) or an antagonist of a receptor which contains as one of its subunits the P2X₃ subunit, such as a P2X_{2/3} heteromeric receptor;
a Ca_{V}2.2 calcium channel blocker (N-type), such as ziconotide; and
a Ca_{V}3.2 calcium channel blocker (T-type), such as ethosuximide.

Pharmaceutical compositions suitable for the delivery of compounds and salts of the present invention and methods for their preparation will be readily apparent to those skilled in the art. Such compositions and methods for their preparation may be found, for example, in ‘Remington's Pharmaceutical Sciences’, 19th Edition (Mack Publishing Company, 1995).

Compounds and salts of the invention intended for pharmaceutical use may be prepared and administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

### Oral Administration

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations, such as tablets, capsules containing particulates, liquids, or powders; lozenges (including liquid-filled), chews; multi- and nano-particulates; gels, solid solution, liposome, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavoring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tableting. The final formulation may comprise one or more layers and may be coated or uncoated; it may even be encapsulated.

The formulation of tablets is discussed in “Pharmaceutical Dosage Forms: Tablets, Vol. 1”, by H. Lieberman and L. Lachman, Marcel Dekker, N.Y., N.Y., 1980 (ISBN 0-8247-6918-X).

The foregoing formulations for the various types of administration discussed above may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Verma et al, Pharmaceutical Technology On-line, 25(2), 1-14 (2001). The use of chewing gum to achieve controlled release is described in WO 00/35298.

### Parenteral Administration

The compounds and salts of the invention may be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) and salts used in the preparation of parenteral solutions may be increased by the use of appropriate formulation techniques, such as the incorporation of solubility-enhancing agents.

Formulations for parenteral administration may be formulated to be immediate and/or modified release. Thus, compounds and salts of the invention may be formulated as a solid, semi-solid, or thixotropic liquid for administration as an implanted depot providing modified release of the active compound. An example of such formulations include drug-coated stents.

### Topical Administration

The compounds and salts of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated [see, for example, Finnin and Morgan, J Pharm Sci, 88 (10), 955-958 (October 1999).] Other means of topical administration include delivery by electroporation, iontophoresis, phonophoresis, sonophoresis and microneedle or needle-free (e.g. Powderject™, Bioject™, etc.) injection.

### Inhaled/Intranasal Administration

The compounds and salts of the invention may also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

A pressurised container, pump, spray, atomizer, or nebuliser may contain a solution or suspension of the compound(s) or salt(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or HPMC), blisters and cartridges for use in an inhaler or insufflator may be formulated to contain a powder mix of the compound or salt of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound or salt of the invention per actuation and the actuation volume may vary from 1 μl to 100 μl. A typical formulation may comprise a compound of formula (I) or salt thereof, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, poly(DL-lactic-coglycolic acid (PGLA). Modified release formulations include delayed-, sustained-, pulsed-, controlled-, targeted and programmed release.

In the case of dry powder inhalers and aerosols, the dosage unit is determined by a prefilled capsule, blister or pocket or by a system that utilises a gravimetrically fed dosing chamber. Units in accordance with the invention are typically arranged to administer a metered dose or “puff” containing from 1 to 5000 μg of the compound or salt. The overall daily dose will typically be in the range 1 μg to 20 mg which may be administered in a single dose or, more usually, as divided doses throughout the day.

### Rectal/Intravaginal Administration

The compounds and salts of the invention may be administered rectally or vaginally, for example, in the form of a suppository, pessary, or enema. Cocoa butter is a traditional suppository base, but various well known alternatives may be used as appropriate.

### Ocular and Aural Administration

The compounds and salts of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid; a cellulosic polymer, for example, hydroxypropylmethylcellulose, hydroxyethylcellulose, or methyl cellulose; or a heteropolysaccharide polymer, for example, gelan gum, may be incorporated together with a preservative, such as benzalkonium chloride. Such formulations may also be delivered by iontophoresis.

### Other Technologies

The compounds and salts of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

For administration to human patients, the total daily dose of the compounds and salts of the invention is typically in the range 0.1 mg to 200 mg depending, of course, on the mode of administration, preferred in the range 1 mg to 100 mg and more preferred in the range 1 mg to 50 mg. The total daily dose may be administered in single or divided doses.

These dosages are based on an average human subject having a weight of about 65 kg to 70 kg. The physician will readily be able to determine doses for subjects whose weight falls outside this range, such as infants and the elderly.

For the above-mentioned therapeutic uses, the dosage administered will, of course, vary with the compound or salt employed, the mode of administration, the treatment desired and the disorder indicated. The total daily dosage of the compound of formula (I)/salt/solvate (active ingredient) will, generally, be in the range from 1 mg to 1 gram, preferably 1 mg to 250 mg, more preferably 10 mg to 100 mg. The total daily dose may be administered in single or divided doses. The present invention also encompasses sustained release compositions.

The pharmaceutical composition may, for example, be in a form suitable for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefor, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regiments for administration of the chemotherapeutic agent are well-known in the relevant art and would be understood to be encompassed by the skilled artisan once provided the teachings disclosed herein.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

For parenteral dosages, this may conveniently be prepared as a solution or as a dry powder requiring dissolution by a pharmacist, medical practitioner or the patient. It may be provided in a bottle or sterile syringe. For example it may be provided as a powder in a multicompartment syringe which allows the dry powder and solvent to be mixed just prior to administration (to aid long-term stability and storage). Syringes could be used which allow multiple doses to be administered from a single device.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations as discussed below. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

A composition of the present invention can be administered by a variety of methods known in the art. The route and/or mode of administration vary depending upon the desired results. The active compounds can be prepared with carriers that protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are described by e.g., Sustained and Controlled Release Drug Delivery Systems, J. R. Robinson, ed., Marcel Dekker, Inc., New York, (1978). Pharmaceutical compositions are preferably manufactured under GMP conditions.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

The precise dosage administered of each active ingredient will vary depending upon any number of factors, including but not limited to, the type of animal and type of disease state being treated, the age of the animal, and the route(s) of administration.

The following non-limiting Preparations and Examples illustrate the preparation of compounds and salts of the present invention. In the Examples and Preparations that are set out below, and in the aforementioned Schemes, the following the abbreviations, definitions and analytical procedures may be referred to. Other abbreviations common in the art are also used. Standard IUPAC nomenclature has been used.

AcOH is acetic acid; aq is aqueous; Bn is benzyl; Boc is tert-butoxycarbonyl; br is broad; tBu is tert-butyl; ° C. is degrees celcius; CDCl₃ is deutero-chloroform; Cs₂CO₃ is cesium carbonate; CsF is cesium fluoride; δ is chemical shift; d is doublet; DCM is dichloromethane/methylene chloride; DIPEA is N-ethyldiisopropylamine, N,N-diisopropylethylamine; DMF is N,N-dimethylformamide; DMSO is dimethyl sulphoxide; EDCI.HCl is 1-(3-dimethylaminopropyl)-3-ethylcarbodiimide hydrochloride; EtOAc is ethyl acetate; EtOH is ethanol; Et₃N is triethylamine; g is gram; HATU is 1-[Bis(dimethylamino)methylene]-1H-1,2,3-triazolo[4,5-b]pyridinium 3-oxid hexafluorophosphate; HBTU is N,N,N′,N′-Tetramethyl-O-(1H-benzotriazol-1-yl)uronium hexafluorophosphate, O-(Benzotriazol-1-yl)-N,N,N′,N′-tetramethyluronium hexafluorophosphate; HCl is hydrochloric acid; HPLC is high pressure liquid chromatography; H₂O is water; H₂O₂ is hydrogen peroxide; HOBt is hydroxybenzotriazole; IMS is industrial methylated spirit; K₂CO₃ is potassium carbonate; KHSO₄ is potassium hydrogen sulphate; L is litre; LCMS is liquid chromatography mass spectrometry (Rt=retention time); LiOH is lithium hydroxide; m is multiplet; M is molar; MeCN is acetonitrile; MeOH is methanol; mg is milligram; MgSO₄ is magnesium sulphate; MHz is mega Hertz; min is minutes; mL is millilitre; mmol is millimole; mol is mole; MS m/z is mass spectrum peak; NaHCO₃ is sodium hydrogencarbonate; NaOH is sodium hydroxide; NH₃ is ammonia; NH₄Cl is ammonium chloride; NH₄HCO₃ is ammonium hydrogen carbonate; NH₄OH is ammonium hydroxide; NMR is nuclear magnetic resonance; Pd/C is palladium on carbon; Pd(dppf)Cl₂ is 1,1-bis(diphenylphosphino)ferrocene-palladium(II)dichloride; Pd(PPh₃)₄ is tetrakis(triphenylphosphine)palladium; pH is power of hydrogen; ppm is parts per million; q is quartet; Rt is retention time; s is singlet; SCX is strong cation exchange; t is triplet; T3P is propylphosphonic anhydride; TBAF is tert-butyl ammonium fluoride; TBDMS is tertbutyldimethylsilyl; TBME is tert-butyl dimethyl ether; TEA is triethylamine; TFA is trifluoroacetic acid; THF is tetrahydrofuran; μL is microlitre and μmol is micromol.

¹H and ¹⁹F Nuclear magnetic resonance (NMR) spectra were in all cases consistent with the proposed structures. Characteristic chemical shifts (δ) are given in parts-per-million downfield from tetramethylsilane (for ¹H-NMR) and upfield from trichloro-fluoro-methane (for ¹⁹F NMR) using conventional abbreviations for designation of major peaks: e.g. s, singlet; d, doublet; t, triplet; q, quartet; m, multiplet; br, broad. The following abbreviations have been used for common solvents: CDCl₃, deuterochloroform; d₆-DMSO, deuterodimethylsulphoxide; and CD₃OD, deuteromethanol. Where appropriate, tautomers may be recorded within the NMR data; and some exchangeable protons may not be visible.

Mass spectra, MS (m/z), were recorded using either electrospray ionisation (ESI) or atmospheric pressure chemical ionisation (APCI).

Where relevant and unless otherwise stated the m/z data provided are for isotopes ¹⁹F, ³⁵Cl, ⁷⁹Br and ¹²⁷I.

Wherein preparative TLC or silica gel chromatography have been used, one skilled in the art may choose any combination of solvents to purify the desired compound. Wherein an SCX-2 column has been used, the eluant conditions are MeOH followed by 7N NH₃ in MeOH.

Wherein reverse phase column chromatography has been used, either acid or basic conditions were employed using a Biotage SNAP KP-C18 silica cartridge:

Acidic conditions: between 0 and 100% of acetonitrile (with 0.1% formic acid) in water (with 0.1% formic acid).
Basic conditions: between 0 and 100% of acetonitrile (with 0.1% ammonia) in water (with 0.1% ammonia, 33% aqueous ammonia used).

Wherein Preparative HPLC has been used, one of the following methods was employed:

### Preparative HPLC Using Acid:

Column: Gemini 5 u C18 110 A 100*21.2 mm 5 micron
Mobile phase A: Water
Mobile phase B: Acetonitrile
Modifier: 0.1% formic acid
Room temperature; 10 minute run time; Initial: 95% A, 5% B to 5% A and 95% B at 7 minutes, hold time 2 minutes, then back to 95% A, 5% B at 9.1 minutes. Flow rate 18 mL/min.

### Detectors

ELSD=Polymer labs PL-ELS 2100
UV=Waters 2487 detector at 225 and 255 nm
Mass spectrometer=Waters ZQ using electrospray ionisation

### Preparative HPLC Using Base:

Column: Gemini 5 u C18 110 A 100*21.2 mm 5 micron
Mobile phase A: Water
Mobile phase B: Acetonitrile
Modifier: 0.1% diethylamine
Room temperature; 10 minute run time; Initial: 95% A, 5% B to 5% A and 95% B at 7 minutes, hold time 2 minutes, then back to 95% A, 5% B at 9.1 minutes. Flow rate 18 mL/min.

### Detectors

ELSD=Polymer labs PL-ELS 2100
UV=Waters 2487 detector at 225 and 255 nm
Mass spectrometer=Waters ZQ using electrospray ionisation

Following Preparative HPLC the following analytical conditions were employed:

### Acidic Analytical Conditions:

Column: Gemini 3 u C18 110 A 50*4.6 mm 3 micron
Mobile phase A: Water
Mobile phase B: Acetonitrile
Modifier: 0.1% formic acid
Room temperature; 5 minute run time; Initial: 95% A, 5% B to 5% A and 95% B at 3 minutes, hold time 1 minutes, then back to 95% A, 5% B at 4.1 minutes. Flow rate 1.5 mL/min.

### Detectors

ELSD=Polymer labs PL-ELS 2100
UV=Waters 2487 detector at 225 and 255 nm
Mass spectrometer=Waters ZQ using electrospray ionisation

### Basic Analytical Conditions:

Column: Gemini 3 u C18 110 A 50*4.6 mm 3 micron
Mobile phase A: Water
Mobile phase B: Acetonitrile
Modifier: 0.1% ammonia
Room temperature; 5 minute run time; Initial: 95% A, 5% B to 5% A and 95% B at 3 minutes, hold time 1 minutes, then back to 95% A, 5% B at 4.1 minutes. Flow rate 1.5 mL/min.

### Detectors

ELSD=Polymer labs PL-ELS 2100
UV=Waters 2487 detector at 225 and 255 nm

Wherein LCMS was employed, LCMS Agilent 1100 with column XBridge analytical C18 5 um 4.6×50 mm was used with one of the four following conditions at 25° C.:

### System 1 Conditions:

Mobile phase A1: 0.05% formic acid in water
Mobile phase B1: 0.05% formic acid in acetonitrile

| Time | A1 | B1 | Flow |
|---|---|---|---|
| (mins) | (%) | (%) | (mL/min) |
| 0.00 | 95 | 5 | 2.00 |
| 3.50 | 5 | 95 | 2.00 |
| 4.50 | 5 | 95 | 2.00 |
| 4.60 | 95 | 5 | 2.00 |

### System 2 Conditions:

Mobile phase A2: 10 mmol ammonia formate in water
Mobile phase B2: acetonitrile

| Time | A2 | B2 | Flow |
|---|---|---|---|
| (mins) | (%) | (%) | (mL/min) |
| 0.00 | 95 | 5 | 2.00 |
| 3.50 | 5 | 95 | 2.00 |
| 4.50 | 5 | 95 | 2.00 |
| 4.60 | 95 | 5 | 2.00 |

### System 3 Conditions:

Mobile phase A: water
Mobile phase B: acetonitrile
Mobile phase C: 10 mmol ammonia formate in water
Mobile phase D: 0.05% formic acid in acetonitrile

| Time | A | B | C | D | Flow |
|---|---|---|---|---|---|
| (mins) | (%) | (%) | (%) | (%) | (mL/min) |
| 0 | 95 | 0 | 0 | 5 | 2 |
| 3.5 | 0 | 95 | 0 | 5 | 2 |
| 4.5 | 0 | 95 | 0 | 5 | 2 |
| 4.6 | 95 | 0 | 0 | 5 | 2 |

### System 4 Conditions:

Mobile phase A: water
Mobile phase B: acetonitrile
Mobile phase C: 10 mmol ammonium formate in water
Mobile phase D: 0.05% formic acid in acetonitrile

| Time | A | B | C | D | Flow |
|---|---|---|---|---|---|
| (mins) | (%) | (%) | (%) | (%) | (mL/min) |
| 0 | 0 | 0 | 100 | 0 | 2 |
| 3.5 | 0 | 95 | 5 | 0 | 2 |
| 4.5 | 0 | 95 | 5 | 0 | 2 |
| 4.6 | 0 | 0 | 100 | 0 | 2 |

### Example 1

### 5-(5-(6-amino-3,5-difluoropyridin-2-yl)-2-chlorobenzamido)-N-cyclopropyl-1-phenyl-1H-pyrazole-3-carboxamide

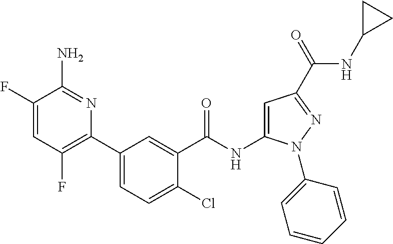 [SMILES: Nc1nc(-c2ccc(Cl)c(C(=O)Nc3cc(C(=O)NC4CC4)nn3-c3ccccc3)c2)c(F)cc1F]

To a solution of 5-(5-(6-amino-3,5-difluoropyridin-2-yl)-2-chlorobenzamido)-1-phenyl-1H-pyrazole-3-carboxylic acid (Example 139, 122 mg, 0.25 mmol) in DMF (2 mL) was added diisopropylethylamine (160 μL, 0.92 mmol) followed by 1-[bis(dimethylamino)methylene]-1H-1,2,3-triazolo[4,5-b]pyridinium 3-oxid hexafluorophosphate (110 mg, 0.30 mmol) and cyclopropylamine (20 μL, 0.29 mmol). The reaction was stirred at room temperature for 1.5 hours. The reaction was partitioned between ethyl acetate (40 mL) and saturated aqueous sodium hydrogen carbonate solution (10 mL). The organic layer was washed with saturated aqueous sodium chloride solution (10 mL), dried over anhydrous sodium sulphate and concentrated in vacuo. The residue was purified using silica gel column chromatography eluting with 50-75% ethyl acetate in heptanes, then dissolved in ethyl acetate (40 mL) and washed with dilute aqueous citric acid (20 mL, 0.01M). The organic layer was then washed with dilute aqueous sodium hydrogen carbonate (20 mL), washed with saturated aqueous sodium chloride solution, dried over anhydrous sodium sulphate and concentrated in vacuo. The residue was dissolved in methanol (2 mL) and water was added to precipitate a solid that was filtered and collected as the title compound (24 mg, 19%).

¹H NMR (400 MHz, DMSO-d₆): δ ppm 0.60-0.66 (m, 4H), 2.84 (m, 1H), 6.38 (br s, 2H), 6.88 (s, 1H), 7.44-7.63 (m, 6H), 7.73 (t, 1H), 7.89-7.90 (m, 2H), 8.27 (m, 1H), 10.77 (s, 1H).

LCMS Rt=2.80 minutes MS m/z 509 [M+H]⁺

### Example 2

### 5-(5-(6-aminopyridin-2-yl)-2-chlorobenzamido)-N-cyclopropyl-1-phenyl-1H-pyrazole-3-carboxamide

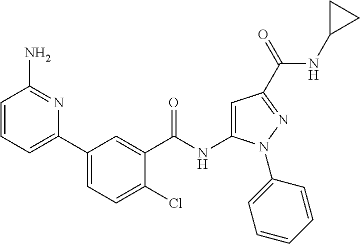 [SMILES: Nc1cccc(-c2ccc(Cl)c(C(=O)Nc3cc(C(=O)NC4CC4)nn3-c3ccccc3)c2)n1]

To a stirred solution of 5-(5-(6-aminopyridin-2-yl)-2-chlorobenzamido)-1-phenyl-1H-pyrazole-3-carboxylic acid (Example 138, 200 mg, 0.46 mmol), cyclopropanamine (262 mg, 4.6 mmol), and triethylamine (209 mg, 2.03 mmol) in DMF (10 mL) was added (O-(7-azabenzotriazol-1-yl)-N,N,N′,N′-tetramethyluronium hexafluorophosphate (354 mg, 0.92 mmol) and the reaction stirred at room temperature for 16 hours. The reaction was quenched with water (10 mL) and extracted into ethyl acetate (3×10 mL). The combined organic layers were dried over magnesium sulphate and concentrated in vacuo. The residue was purified by preparative HPLC to afford the title compound as a colourless solid (19 mg, 9%).

¹H NMR (400 MHz, DMSO-d₆): δ ppm 0.56-0.68 (m, 4H), 2.78-2.88 (m, 1H), 6.05-6.10 (br s, 1H), 6.42-6.47 (d, 1H), 6.89 (s, 1H), 7.06-7.14 (d, 1H), 7.42-7.64 (m, 7H), 8.02-8.09 (m, 2H), 8.27-8.32 (m, 1H).

LCMS Rt=2.90 minutes MS m/z 473 [M+H]⁺

The following Examples were prepared according to the Method described by Example 1 or Example 2 using 5-(5-(6-aminopyridin-2-yl)-2-chlorobenzamido)-1-phenyl-1H-pyrazole-3-carboxylic acid (Example 138) and the appropriate amine as described below. The crude residues were purified as above or according to the purification method described below:

Purification Method A: Silica gel column chromatography eluting with 5% MeOH in DCM.
Purification Method B: Silica gel column chromatography eluting with EtOAc.
Purification Method C: Silica gel column chromatography eluting with 60% heptanes in acetone.

| Example | | | |
|---|---|---|---|
| Number | Name | Structure | Data/Amine/PM |
| 3 | 5-(5-(6-aminopyridin-2- yl)-2-chlorobenzamido)- N,N-dimethyl-1-phenyl- 1H-pyrazole-3- carboxamide | 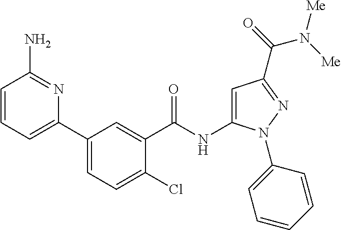 [SMILES: CN(C)C(=O)c1cc(NC(=O)c2cc(-c3cccc(N)n3)ccc2Cl)n(-c2ccccc2)n1] | ¹H NMR (400 MHz, DMSO- d₆): δ ppm 3.00 (s, 3H), 3.35 (s, 3H), 6.80 (m, 1H), 7.00 (m, 1H), 7.25 (m, 1H), 7.40-7.60 (m, 5H), 7.80 (m, 1H), 7.95 (m, 3H), 10.80 (s, 1H). LCMS Rt = 1.83 minutes MS m/z 461 [M + H]⁺ Using dimethylamine. |
| 4 | 5-(5-(6-aminopyridin-2- yl)-2-chlorobenzamido)- N-(2-hydroxyethyl)-1- phenyl-1H-pyrazole-3- carboxamide | 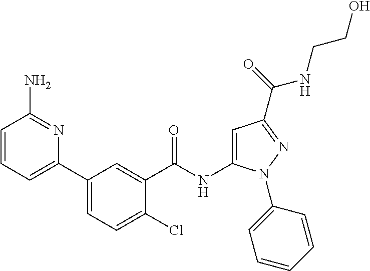 [SMILES: Nc1cccc(-c2ccc(Cl)c(C(=O)Nc3cc(C(=O)NCCO)nn3-c3ccccc3)c2)n1] | ¹H NMR (400 MHz, CDCl₃): δ ppm 3.30 (m, 2H), 3.50 (m, 2H), 4.75 (m, 1H). 6.10 (m, 1H), 6.45 (m, 1H), 6.90 (m, 1H), 7.10 (m, 1H), 7.25 (m, 2H), 7.40-7.80 (m, 7H), 8.00-8.20 (m, 3H), 10.80 (s, 1H). LCMS Rt = 1.83 minutes MS m/z 477 [M + H]⁺ Using ethanolamine. |
| 5 | 5-(5-(6-aminopyridin-2- yl)-2-chlorobenzamido)- N-((3-hydroxyoxetan-3- yl)methyl)-1-phenyl-1H- pyrazole-3-carboxamide | 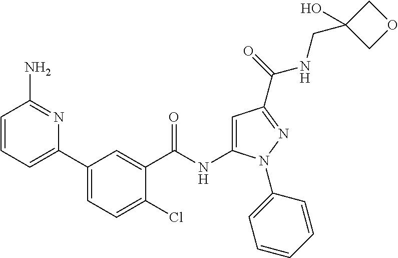 [SMILES: Nc1cccc(-c2ccc(Cl)c(C(=O)Nc3cc(C(=O)NCC4(O)COC4)nn3-c3ccccc3)c2)n1] | ¹H NMR (400 MHz, MeOH- d₄): δ ppm 3.75-3.81 (m, 2H), 4.54-4.59 (m. 2H), 4.60-4.64 (m, 2H), 6.61- 6.66 (d, 1H), 7.03-7.09 (m, 2H), 7.43-7.67 (m, 9H), 7.92-7.98 (m, 1H), 7.98- 8.02 (m, 1H), 8.33-8.42 (m, 1H), 8.66-8.71 (m, 1H). LCMS Rt = 2.42 minutes MS m/z 519 [M + H]⁺ Using 3- (aminomethyl)oxetan-3-ol. PM A. |
| 6 | 5-(5-(6-aminopyridin-2- yl)-2-chlorobenzamido)- N-(oxetan-3-yl)-1-phenyl- 1H-pyrazole-3- carboxamide | 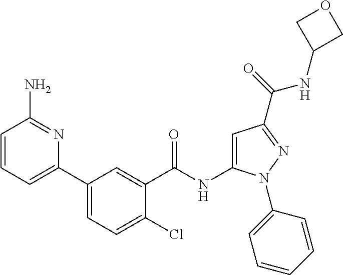 [SMILES: Nc1cccc(-c2ccc(Cl)c(C(=O)Nc3cc(C(=O)NC4COC4)nn3-c3ccccc3)c2)n1] | ¹H NMR (400 MHz, CDCl₃): δ ppm 4.63 (t, 2H), 4.72 (t, 1H), 5.02 (m, 1H), 6.06 (s, 2H), 6.45 (d, 1H), 6.93 (s, 1H), 7.08 (d, 1H), 7.45-7.65 (m, 7H), 8.03-8.08 (m, 2H), 9.02 (d, 1H), 10.77 (s, 1H). LCMS Rt = 2.52 minutes MS m/z 489 [M + H]⁺ Using oxetan-3-amine. PM B. |
| 7 | Racemic-5-(5-(6- aminopyridin-2-yl)-2- chlorobenzamido)-N- (oxetan-2-ylmethyl)-1- phenyl-1H-pyrazole-3- carboxamide | 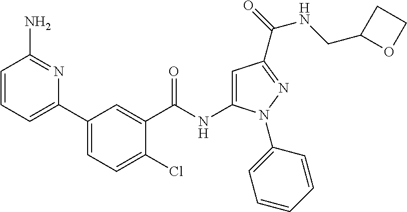 [SMILES: Nc1cccc(-c2ccc(Cl)c(C(=O)Nc3cc(C(=O)NCC4CCO4)nn3-c3ccccc3)c2)n1] | ¹H NMR (400 MHz, CDCl₃): δ ppm 2.50 (m, 1H). 2.70 (m. 1H), 3.63 (dd, 1H), 3.72 (dd, 1H), 4.57 (m, 1H), 4.68 (m, 1H), 5.02 (m, 1H), 6.57 (d. 1H), 7.05 (s, 2H). 7.50- 7.59 (m, 5H), 7.66 (d, 2H), 7.99 (m, 2H). LCMS Rt = 2.56 minutes MS m/z 503 [M + H]+ Using oxetan-2- ylmethanamine. PMA. |
| 8 | 5-(5-(6-aminopyridin-2- yl)-2-chlorobenzamido)- N-(oxetan-3-ylmethyl)-1- phenyl-1H-pyrazole-3- carboxamide | 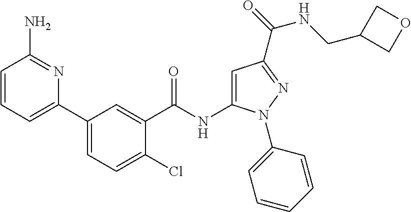 [SMILES: Nc1cccc(-c2ccc(Cl)c(C(=O)Nc3cc(C(=O)NCC4COC4)nn3-c3ccccc3)c2)n1] | ¹H NMR (400MHz, DMSO- d₆): δ ppm 3.20-3.13(m, 1H), 3.55 (t, 2H), 4.37 (t, 2H), 4.63 (q, 2H), 6.08 (s, 2H), 6.47 (d, 1H), 6.92 (s, 1H), 7.11 (d, 1H), 7.64-7.46 (m, 7H), 8.06 (d, 1H), 8.09 (s, 1H), 8.55 (t, 1H), 10.78 (s, 1H). LCMS Rt = 2.74 minutes MS m/z 503 [M + H]⁺ Using oxetan-3- ylmethanamine. PM C. |

The following Examples were prepared according to the Method described by Example 1 or Example 2 using 5-(5-(6-amino-3,5-difluoropyridin-2-yl)-2-chlorobenzamido)-1-phenyl-1H-pyrazole-3-carboxylic acid (Example 139) or 5-(5-(6-amino-3-fluoropyridin-2-yl)-2-chlorobenzamido)-1-phenyl-1H-pyrazole-3-carboxylic acid (Example 140) and the appropriate amine as described below. The crude residues were purified as above or according to the purification method described below:

Purification Method A: Elution through an SCX-2 column using ammonia in MeOH followed by silica gel column chromatography eluting with 30-60% or 50-100% EtOAc in heptanes.
Purification Method B: Elution through an SCX-2 column using ammonia in MeOH followed by silica gel column chromatography eluting with 5% MeOH in DCM.
Purification Method C: Elution through an SCX-2 column using ammonia in MeOH followed by trituration with MeCN or DCM.
Purification Method D: Reverse phase column chromatography

| Example | | | |
|---|---|---|---|
| Number | Name | Structure | Data/Amine/PM |
| 9 | 5-{[5-(6-amino-3,5- difluoropyridin-2-yl)-2- chlorobenzoyl]amino}-N- (2-hydroxy-2- methylpropyl)-1-phenyl- 1H-pyrazole-3- carboxamide | 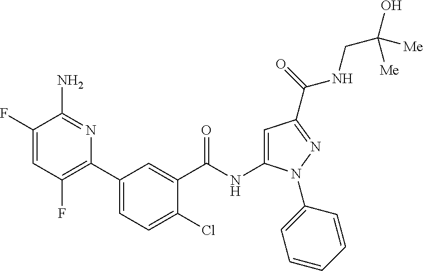 [SMILES: CC(C)(O)CNC(=O)c1cc(NC(=O)c2cc(-c3nc(N)c(F)cc3F)ccc2Cl)n(-c2ccccc2)n1] | ¹H NMR (400 MHz, MeOH- d₄): δ ppm 1.18 (s, 6H), 3.41 (s, 2H), 7.03 (s, 1H), 7.42 (t, 1H), 7.47-7.57 (m, 4H), 7.63-7.64 (m, 2H), 7.97-8.00 (m, 2H). LCMS Rt = 2.73 minutes MS m/z 541 [M + H]⁺ Using 1-amino-2- methylpropan-2-ol. PM A. |
| 10 | (R)-5-(5-(6-amino-3,5- difluoropyridin-2-yl)-2- chlorobenzamido)-N-(2- hydroxypropyl)-1-phenyl- 1H-pyrazole-3- carboxamide | 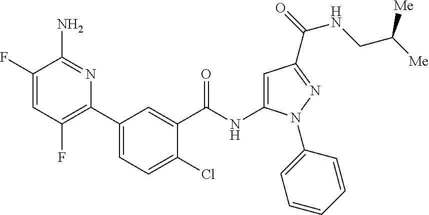 | ¹H NMR (400 MHz, DMSO- d₆): δ ppm 1.05 (d, 3H), 3.12-3.18 (m, 1H), 3.22-3.30 (m, 1H), 3.74-3.79 (m, 1H), 6.38 (br s, 2H), 6.90 (s, 1H), 7.46-7.63 (m, 6H), 7.73 (t, 1H), 7.89-7.91 (m, 2H), 8.05 (t, 1H), 10.79 (s, 1H). LCMS Rt = 2.55 minutes MS m/z 527 [M + H]⁺ Using (R)-1-aminopropan-2- ol. PM B. |
| 11 | 5-(5-(6-amino-3,5- difluoropyridin-2-yl)-2- chlorobenzamido)-N- (oxetan-3-yl)-1-phenyl- 1H-pyrazole-3- carboxamide | 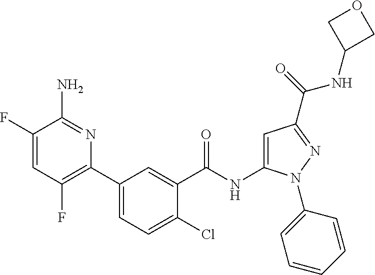 [SMILES: Nc1nc(-c2ccc(Cl)c(C(=O)Nc3cc(C(=O)NC4COC4)nn3-c3ccccc3)c2)c(F)cc1F] | ¹H NMR (400 MHz, DMSO- d6): δ ppm 3.43-3.48 (m, 1H), 3.55-3.60 (m, 1H), 4.22-4.31 (m, 2H), 4.40 (m, 1H), 4.83 (t, 1H), 6.38 (br s, 2H), 6.89 (s, 1H), 7.44-7.76 (m, 6H), 7.73 (t, 1H), 7.89- 7.91 (m, 2H), 10.82 (s, 1H). MS m/z 525 [M + H]⁺ Using oxetan-3-amine. PM C. |
| 12 | 5-(5-(6-amino-3,5- difluoropyridin-2-yl)-2- chlorobenzamido)-N- ((1s,4s)-4- hydroxycyclohexyl)-1- phenyl-1H-pyrazole-3- carboxamide | 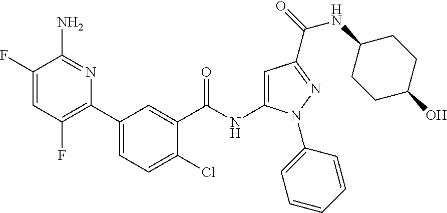 [SMILES: Nc1nc(-c2ccc(Cl)c(C(=O)Nc3cc(C(=O)N[C@H]4CC[C@@H](O)CC4)nn3-c3ccccc3)c2)c(F)cc1F] | ¹H NMR (400 MHz, MeOH- d₄): δ ppm 1.66-1.87 (m, 8H), 3.89 (m, 1H), 3.94-3.98 (m, 1H), 7.02 (s, 1H), 7.41 (t, 1H), 7.49-7.56 (m, 4H), 7.62-7.64 (m, 2H), 7.97-7.99 (m, 2H). LCMS Rt = 2.77 minutes MS m/z 567 [M + H]⁺ Using (1s,4s)-cyclohexane- 1,4-diamine. PM A. |
| 13 | 5-(5-(6-amino-3,5- difluoropyridin-2-yl)-2- chlorobenzamido)-N-(3- hydroxypropyl)-1-phenyl- 1H-pyrazole-3- carboxamide | 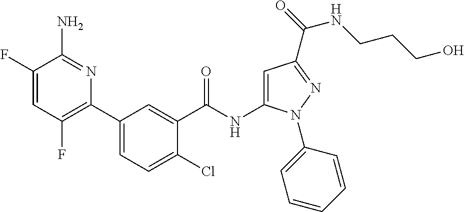 [SMILES: Nc1nc(-c2ccc(Cl)c(C(=O)Nc3cc(C(=O)NCCCO)nn3-c3ccccc3)c2)c(F)cc1F] | ¹H NMR (400 MHz, CDCl₃): δ ppm 1.73 (t, 2H), 3.56 (q, 2H), 3.64 (t, 2H), 7.29 (s, 1H), 7.40 (d, 1H), 7.48-7.55 (m, 6H), 7.92 (d, 1H), 8.34 (s, 1H), 8.56 (s, 1H). LCMS Rt = 2.71 minutes MS m/z 527 [M + H]⁺ Using 3-amino-propan-1-ol. PM B. |
| 14 | (S)-5-(5-(6-amino-3,5- difluoropyridin-2-yl)-2- chlorobenzamido)-1- phenyl-N- (tetrahydrofuran-3-yl)- 1H-pyrazole-3- carboxamide | 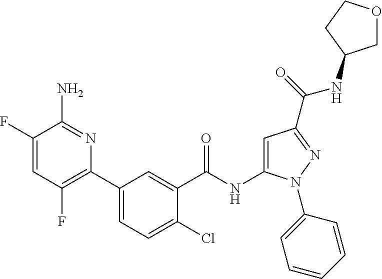 [SMILES: Nc1nc(-c2ccc(Cl)c(C(=O)Nc3cc(C(=O)N[C@H]4CCOC4)nn3-c3ccccc3)c2)c(F)cc1F] | ¹H NMR (400 MHz, MeOH- d₄): δ ppm 1.98-12.05 (m, 1H), 2.26-2.35 (m, 1H), 3.74 (dd, 1H), 3.81-3.86 (m, 1H), 3.94-4.01 (m, 2H), 4.60-4.64 (m, 1H), 7.03 (s, 1H), 7.41 (t, 1H), 7.47-7.57 (m, 4H), 7.62-7.64 (m, 2H), 7.97-7.99 (m, 2H). LCMS Rt = 2.94 minutes MS m/z 539 [M + H]⁺ Using (S)-tetrahydrofuran-3- amine. PM A. |
| 15 | (S)-5-(5-(6-amino-3,5- difluoropyridin-2-yl)-2- chlorobenzamido)-N-(2- hydroxypropyl)-1-phenyl- 1H-pyrazole-3- carboxamide | 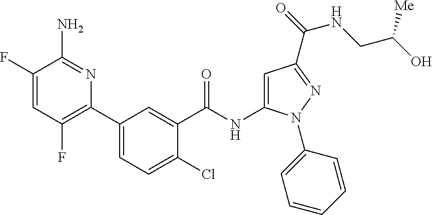 [SMILES: C[C@H](O)CNC(=O)c1cc(NC(=O)c2cc(-c3nc(N)c(F)cc3F)ccc2Cl)n(-c2ccccc2)n1] | ¹H NMR (400 MHz, MeOH- d₄): δ ppm 1.20 (d, 3H), 3.36 (m, 1H), 3.47 (m, 1H), 3.96 (m, 1H), 7.02 (s, 1H), 7.41 (t, 1H), 7.47-7.57 (m, 4H), 7.62-7.64 (m, 2H), 7.97-8.00 (m, 2H). LCMS Rt = 2.65 minutes MS m/z 527 [M + H]⁺ Using (S)-1-aminopropan-2- ol. PM A. |
| 16 | 5-(5-(6-amino-3- fluoropyridin-2-yl)-2- chlorobenzamido)-N-(2- hydroxy-2-methylpropyl)- 1-phenyl-1H-pyrazole-3- carboxamide | 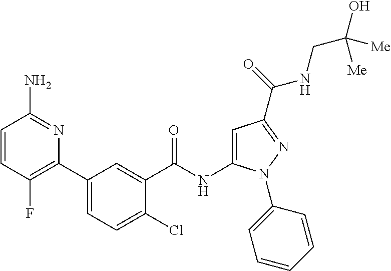 [SMILES: CC(C)(O)CNC(=O)c1cc(NC(=O)c2cc(-c3nc(N)ccc3F)ccc2Cl)n(-c2ccccc2)n1] | ¹H NMR (400 MHz, DMSO- d₆): δ ppm 1.11 (s, 6H), 3.26 (d, 2H), 4.64 (s, 1H), 6.07 (s, 2H), 6.51 (dd, 1H), 6.93 (s, 1H), 7.44-7.64 (m, 7H), 7.85 (t, 1H), 7.95-7.96 (m, 2H), 10.82 (s, 1H). LCMS Rt = 2.62 minutes MS m/z 523 [M + H]⁺ Using 1-amino-2- methylpropan-2-ol. PM C. |
| 17 | 5-(5-(6-amino-3,5- difluoropyridin-2-yl)-2- chlorobenzamido)-N- ((1r,4r)-4- hydroxycyclohexyl)-1- phenyl-1H-pyrazole-3- carboxamide | 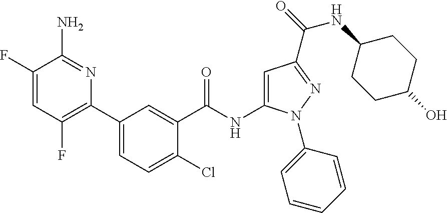 | ¹H NMR (400 MHz, MeOH- d₄): δ ppm 1.36-1.52 (m, 4H), 1.98-2.03 (m, 4H), 3.54-3.59 (m, 1H), 3.84-3.89 (m, 1H), 7.00 (s, 1H), 7.41 (t, 1H), 7.46-7.57 (m, 4H), 7.61-7.63 (m, 2H), 7.97-7.99 (m, 2H). LCMS Rt = 2.78 minutes MS m/z 567 [M + H]⁺ Using (1r,4r)-4-amino cyclohexan-1-ol. PM A. |
| 18 | Racemic-5-(5-(6-amino- 3,5-difluoropyridin-2-yl)- 2-chlorobenzamido)-N- (1-hydroxypropan-2-yl)- 1-phenyl-1H-pyrazole-3- carboxamide | 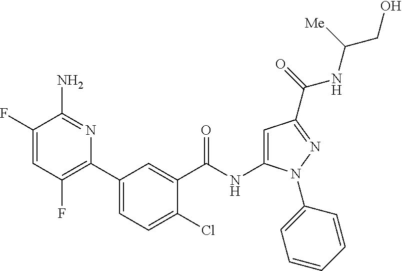 [SMILES: CC(CO)NC(=O)c1cc(NC(=O)c2cc(-c3nc(N)c(F)cc3F)ccc2Cl)n(-c2ccccc2)n1] | ¹H NMR (400 MHz, DMSO- d₆): δ ppm 1.14 (d, 3H), 3.35-3.50 (m, 2H), 3.97-4.04 (m, 1H), 4.76-4.79 (t, 1H), 6.40 (br s, 2H), 6.92 (s, 1H), 7.40-7.90 (m, 9H), 10.80 (s, 1H). MS m/z 527 [M + H]⁺ Using racemic-2- aminopropan-1-ol. PM D. |
| 19 | (R)-5-(5-(6-amino-3,5- difluoropyridin-2-yl)-2- chlorobenzamido)-N-(1- hydroxypropan-2-yl)-1- phenyl-1H-pyrazole-3- carboxamide | 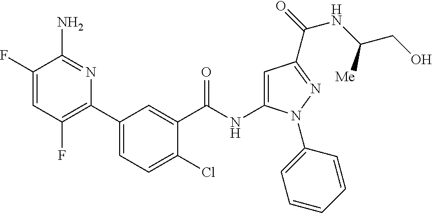 [SMILES: C[C@H](CO)NC(=O)c1cc(NC(=O)c2cc(-c3nc(N)c(F)cc3F)ccc2Cl)n(-c2ccccc2)n1] | ¹H NMR (400 MHz, MeOH- d₄): δ ppm 1.23 (d, 3H), 3.60 (m, 2H), 4.15-4.22 (m, 1H), 7.01 (s, 1H), 7.40-7.63 (m, 7H), 7.98-8.01 (m, 2H), LCMS Rt = 2.66 minutes MS m/z 527 [M + H]⁺ Using (R)-2-aminopropan-1- ol. PM A. |
| 20 | (S)-5-(5-(6-amino-3,5- difluoropyridin-2-yl)-2- chlorobenzamido)-N-(1- hydroxypropan-2-yl)-1- phenyl-1H-pyrazole-3- carboxamide | 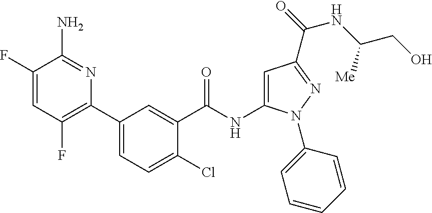 [SMILES: C[C@@H](CO)NC(=O)c1cc(NC(=O)c2cc(-c3nc(N)c(F)cc3F)ccc2Cl)n(-c2ccccc2)n1] | ¹H NMR (400 MHz, MeOH- d₄): δ ppm 1.23 (d, 3H), 3.60 (m, 2H), 4.15-4.22 (m, 1H), 7.01 (s, 1H), 7.40-7.63 (m, 7H), 7.98-8.01 (m, 2H), LCMS Rt = 2.67 minutes MS m/z 527 [M + H]⁺ Using (S)-2-aminopropan-1- ol. PM A. |
| 21 | 5-{[5-(6-amino-3,5- difluoropyridin-2-yl)-2- chlorobenzoyl]amino}-N- (oxetan-3-ylmethyl)-1- phenyl-1H-pyrazole-3- carboxamide | 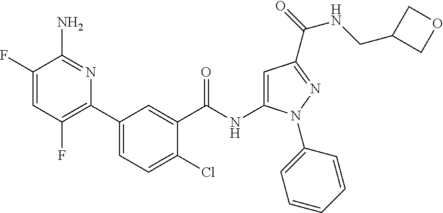 [SMILES: Nc1nc(-c2ccc(Cl)c(C(=O)Nc3cc(C(=O)NCC4COC4)nn3-c3ccccc3)c2)c(F)cc1F] | ¹H NMR (400 MHz, MeOH- d₄): δ ppm 2.17-2.26 (m, 1H), 3.35-3.40 (m, 1H), 3.57-3.66 (m, 3H), 4.19-4.24 (m, 1H), 4.48-4.51 (m, 1H), 6.94 (s, 1H), 7.39-7.61 (m, 7H), 7.96-7.99 (m, 2H). LCMS Rt = 2.65 minutes MS m/z 539 [M + H]⁺ Using oxetan-3- ylmethanamine. PM B. |
| 22 | 5-{[5-(6-amino-3,5- difluoropyridin-2-yl)-2- chlorobenzoyl]amino}-N- (1-hydroxy-2- methylpropan-2-yl)-1- phenyl-1H-pyrazole-3- carboxamide | 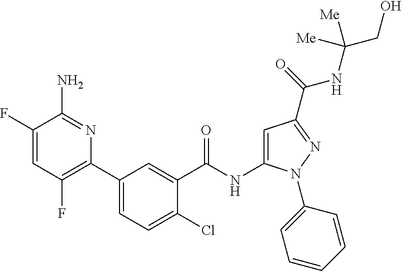 [SMILES: CC(C)(CO)NC(=O)c1cc(NC(=O)c2cc(-c3nc(N)c(F)cc3F)ccc2Cl)n(-c2ccccc2)n1] | ¹H NMR (400 MHz, MeOH- d₄): δ ppm 1.43 (s, 6H), 3.65 (s, 2H), 6.98 (s, 1H), 7.39- 7.61 (m, 7H), 7.96-7.99 (m, 2H). MS m/z 541 [M + H]⁺ Using 2-amino-2- methylpropan-1-ol. PM B. |
| 23 | (S)-5-(5-(6-amino-3,5- difluoropyridin-2-yl)-2- chlorobenzamido)-N-(2- hydroxypropyl)-1-phenyl- 1H-pyrazole-3- carboxamide | 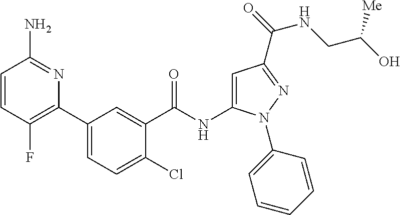 [SMILES: C[C@H](O)CNC(=O)c1cc(NC(=O)c2cc(-c3nc(N)ccc3F)ccc2Cl)n(-c2ccccc2)n1] | ¹H NMR (400 MHz, MeOH- d₄): δ ppm 1.21 (d, 3H), 3.31 (m, 1H), 3.47 (dd, 1H), 3.96 (m, 1H), 6.58 (dd, 1H), 7.03 (s, 1H), 7.38 (dd, 1H), 7.47- 7.61 (m, 4H), 7.62-7.65 (m, 2H), 7.98-8.00 (m, 2H). LCMS Rt = 2.60 minutes MS m/z 509 [M + H]⁺ Using (S)-1-aminopropan-2- ol at 50° C. PM A. |
| 24 | 5-(5-(6-amino-3,5- difluoropyridin-2-yl)-2- chlorobenzamido)-N- ((1r,3r)-3- (hydroxymethyl)cyclo- butyl)-1-phenyl-1H- pyrazole-3-carboxamide | 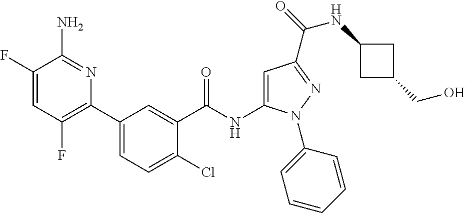 [SMILES: Nc1nc(-c2ccc(Cl)c(C(=O)Nc3cc(C(=O)N[C@H]4C[C@H](CO)C4)nn3-c3ccccc3)c2)c(F)cc1F] | ¹H NMR (400 MHz, MeOH- d₄): δ ppm 2.20-2.30 (m, 4H), 2.35-2.45 (m, 1H), 3.30 (m, 2H), 4.60 (m ,1H), 7.00 (s, 1H), 7.40-7.65 (m, 7H), 8.00 (m, 2H). LCMS Rt = 2.70 minutes MS m/z 553 [M + H]⁺ Using ((1r,3r)-3- aminocyclobutyl)methanol. |
| 25 | 5-(5-(6-amino-3,5- difluoropyridin-2-yl)-2- chlorobenzamido)-N- ((1s,3s)-3- (hydroxymethyl)cyclo- butyl)-1-phenyl-1H- pyrazole-3-carboxamide | 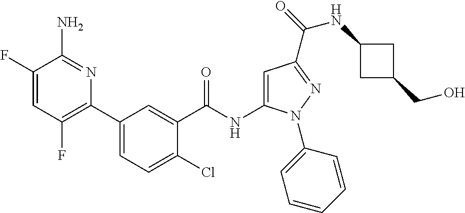 [SMILES: Nc1nc(-c2ccc(Cl)c(C(=O)Nc3cc(C(=O)N[C@H]4C[C@@H](CO)C4)nn3-c3ccccc3)c2)c(F)cc1F] | ¹H NMR (400 MHz, MeOH- d₄): δ ppm 1.80 (m, 2H), 2.20 (m, 1H), 2.40 (m, 2H), 3.55 (m, 2H), 4.40 (m, 1H), 7.00 (s, 1H), 7.40-7.65 (m, 7H), 8.00 (m, 2H). LCMS Rt = 2.69 minutes MS m/z 553 [M + H]⁺ Using ((1s,3s)-3- aminocyclobutyl)methanol. |

The following Examples were prepared according to the Method described by Example 1 using the appropriate carboxylic acid and the appropriate amine as described below. The crude residues were purified as above or according to the purification method described below:

| Example | | | |
|---|---|---|---|
| Number | Name | Structure | Data/Amine/PM |
| 26 | 5-(5-(6-amino-3- chloropyridin-2-yl)-2- chlorobenzamido)-N- (2-hydroxy-2- methylpropyl)-1- phenyl-1H-pyrazole-3- carboxamide | 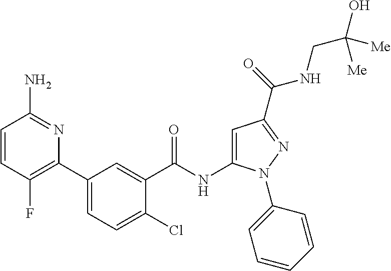 [SMILES: CC(C)(O)CNC(=O)c1cc(NC(=O)c2cc(-c3nc(N)ccc3F)ccc2Cl)n(-c2ccccc2)n1] | ¹H NMR (400 MHz, MeOH-d₄): δ ppm 1.24 (s, 6H), 3.41 (s, 2H), 6.57 (d, 1H), 7.02 (s, 1H), 7.46- 7.56 (m, 5H), 7.61-7.63 (m, 2H), 7.71-7.75 (m, 2H). LCMS Rt = 2.54 minutes MS m/z 539 [M + H]⁺ Using 5-(5-(6-amino-3- chloropyridin-2-yl)-2- chlorobenzamido)-1-phenyl-1H- pyrazole-3-carboxylic acid (Example 141) and 1-amino-2- methylpropan-2-ol. PM A. |
| 27 | 5-(5-(6-amino-5- fluoropyridin-2-yl)-2- chlorobenzamido)-N- cyclopropyl-1-phenyl- 1H-pyrazole-3- carboxamide | 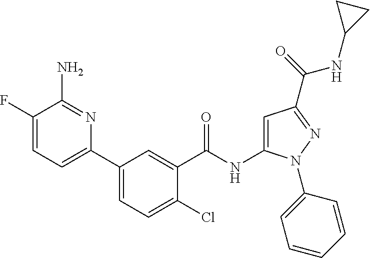 [SMILES: Nc1nc(-c2ccc(Cl)c(C(=O)Nc3cc(C(=O)NC4CC4)nn3-c3ccccc3)c2)ccc1F] | ¹H NMR (400 MHz, DMSO-d₆): δ ppm 0.67 (m, 2H), 0.81 (q, 2H), 2.86 (m, 1H), 7.02 (s, 1H), 7.05 (dd, 1H), 7.34 (t, 1H), 7.50 (d, 2H), 7.55 (t, 2H), 7.64 (d, 2H), 7.97 (d, 1H), 8.02 (s, 1H). LCMS Rt = 2.84 minutes MS m/z 491 [M + H]⁺ Using 5-(5-(6-amino-5- fluoropyridin-2-yl)-2- chlorobenzamido)-1-phenyl-1H- pyrazole-3-carboxylic acid (Example 142) and cylcopropylamine. PM D. |

### Example 28

### Racemic-5-(5-(6-amino-3,5-difluoropyridin-2-yl)-2-chlorobenzamido)-N-(2-hydroxypropyl)-1-phenyl-1H-pyrazole-3-carboxamide

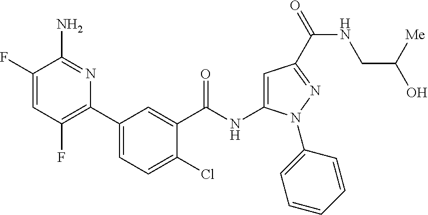 [SMILES: CC(O)CNC(=O)c1cc(NC(=O)c2cc(-c3nc(N)c(F)cc3F)ccc2Cl)n(-c2ccccc2)n1]

To a solution of 5-(5-(6-amino-3,5-difluoropyridin-2-yl)-2-chlorobenzamido)-1-phenyl-1H-pyrazole-3-carboxylic acid (Example 139, 100 mg, 0.21 mmol) in DMF (1.5 mL) was added N,N-diisopropylethylamine (0.18 mL, 1.05 mmol), 1-[bis(dimethylamino)methylene]-1H-1,2,3-triazolo[4,5-b]pyridinium 3-oxid hexafluorophosphate (104 mg, 0.27 mmol) and racemic-1-aminopropan-2-ol (17 mg, 0.22 mmol). The reaction was stirred at room temperature for 3 hours. Water (5 mL) was added followed by EtOAc (10 mL). The organic layer was washed with water (3×5 mL), dried over MgSO₄ and concentrated in vacuo. The residue was purified using reverse phase column chromatography to afford the title compound (60 mg, 54%).

¹H NMR (400 MHz, DMSO-d₆): δ ppm 1.06 (d, 3H), 3.17 (m, 1H), 3.26 (m, 1H), 3.78 (m, 1H), 4.78 (d, 1H), 6.40 (s, 2H), 6.92 (s, 1H), 7.48 (d, 1H), 7.56 (m, 5H), 7.75 (t, 1H), 7.91 (s, 2H), 8.07 (s, 1H), 10.81 (s, 1H).

LCMS Rt=2.65 minutes MS m/z 527 [M+H]⁺

### Library Protocol 1

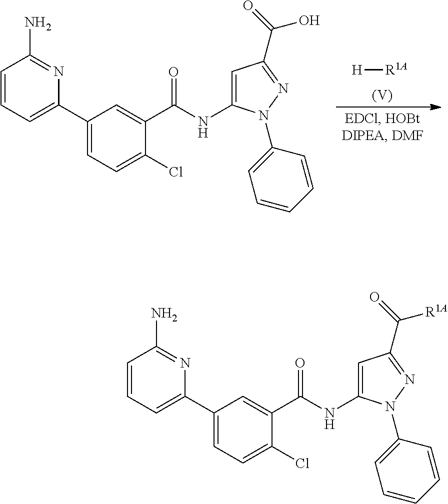

To a 0.5M solution of amines of formula (V) in DMF (500 uL, 250 umol) was added a 0.25M solution of 5-(5-(6-aminopyridin-2-yl)-2-chlorobenzamido)-1-phenyl-1H-pyrazole-3-carboxylic acid (Example 138, 500 uL, 125 umol) in DMF followed by EDCI (60 mg, 312.5 umol), HOBt (26 mg, 187.5 umol) and DIPEA (57 uL, 312.5 umol). The reaction was shaken at 80° C. for 16 hours. The reaction was cooled and purified using preparative HPLC as described below:

### Preparative HPLC:

Column: YMC Triart C18 (250 mm×20 mm, 5 u); using acetonitrile-water (20 mM NH₄HCO₃) from between 10-75% organic; gradient time 18 minutes; hold time 2 minutes; flow rate 20 mL/min.

### LCMS QC:

Column: RESTEK C18 (30 mm×2.1 mm, 3 u); mobile phase A: 0.05% formic acid in water, mobile phase B: MeCN; from 2% B to 10% B at 1 minute, to 98% B at 2 minutes then back to 2% B at 2.90 minutes, flow rate 1.5 mL/min.

| Example | | | |
|---|---|---|---|
| Number | Name | Structure | Data/Amine |
| 29 | 5-{[5-(6-aminopyridin-2- yl)-2- chlorobenzoyl]amino}-N- ethyl-1-phenyl-1H- pyrazole-3-carboxamide | 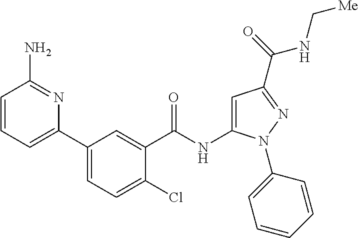 [SMILES: CCNC(=O)c1cc(NC(=O)c2cc(-c3cccc(N)n3)ccc2Cl)n(-c2ccccc2)n1] | LCMS Rt = 1.38 minutes MS m/z 461 [M + H]⁺ Ethylamine |
| 30 | 5-{[5-(6-aminopyridin-2- yl)-2- chlorobenzoyl]amino}-N- [3- (hydroxymethyl)oxetan- 3-yl]-1-phenyl-1H- pyrazole-3-carboxamide | 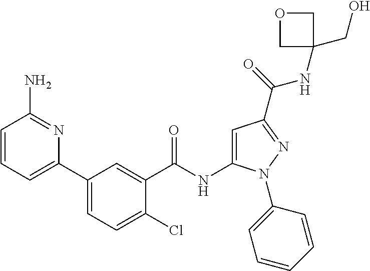 [SMILES: Nc1cccc(-c2ccc(Cl)c(C(=O)Nc3cc(C(=O)NC4(CO)COC4)nn3-c3ccccc3)c2)n1] | LCMS Rt = 1.32 minutes MS m/z 519 [M + H]⁺ (3-Amino-oxetan-3- yl)methanol |
| 31 | 5-{[5-(6-aminopyridin-2- yl)-2- chlorobenzoyl]amino}-1- phenyl-N-[2-(1H-1,2,4- triazol-1-yl)ethyl]-1H- pyrazole-3-carboxamide | 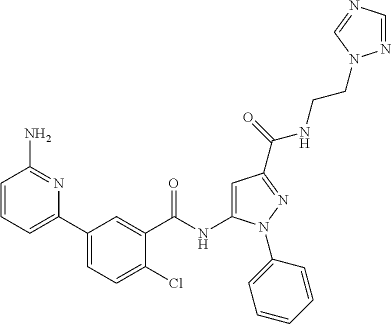 [SMILES: Nc1cccc(-c2ccc(Cl)c(C(=O)Nc3cc(C(=O)NCCn4cncn4)nn3-c3ccccc3)c2)n1] | LCMS Rt = 1.35 minutes MS m/z 528 [M + H]⁺ 2-(1H-1,2,4-Triazol-1- yl)ethan-1-amine |
| 32 | (R)-5-{[5-(6- aminopyridin-2-yl)-2- chlorobenzoyl]amino}-N- (2-hydroxypropyl)-1- phenyl-1H-pyrazole-3- carboxamide | 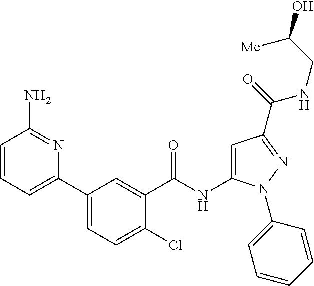 [SMILES: C[C@@H](O)CNC(=O)c1cc(NC(=O)c2cc(-c3cccc(N)n3)ccc2Cl)n(-c2ccccc2)n1] | LCMS Rt = 1.34 minutes MS m/z 491 [M + H]⁺ (R)-1-Aminopropan-2-ol |
| 33 | 5-{[5-(6-aminopyridin-2- yl)-2- chlorobenzoyl]amino}-N- (1H-imidazol-2-ylmethyl)- 1-phenyl-1H-pyrazole-3- carboxamide | 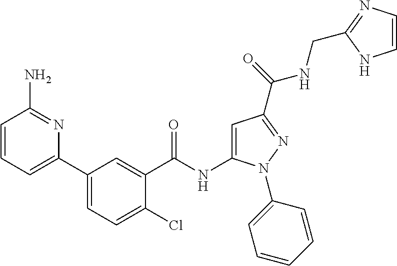 [SMILES: Nc1cccc(-c2ccc(Cl)c(C(=O)Nc3cc(C(=O)NCc4ncc[nH]4)nn3-c3ccccc3)c2)n1] | LCMS Rt = 1.24 minutes MS m/z 511 [M − H]⁻ (1H-imidazol-2- yl)methanamine |
| 34 | 5-{[5-(6-aminopyridin-2- yl)-2- chlorobenzoyl]amino}-N- (3-methyloxetan-3-yl)-1- phenyl-1H-pyrazole-3- carboxamide | 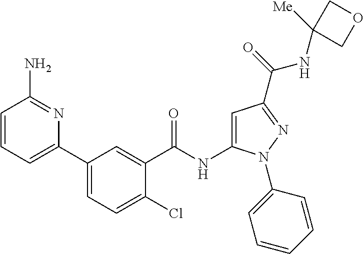 [SMILES: CC1(NC(=O)c2cc(NC(=O)c3cc(-c4cccc(N)n4)ccc3Cl)n(-c3ccccc3)n2)COC1] | LCMS Rt = 1.37 minutes MS m/z 503 [M + H]⁺ 3-Methyloxetan-3-amine |
| 35 | 5-(6-aminopyridin-2-yl)- 2-chloro-N-{3-[(3- hydroxyazetidin-1- yl)carbonyl]-1-phenyl- 1H-pyrazol-5- yl}benzamide | 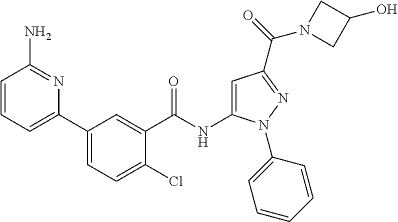 [SMILES: Nc1cccc(-c2ccc(Cl)c(C(=O)Nc3cc(C(=O)N4CC(O)C4)nn3-c3ccccc3)c2)n1] | LCMS Rt = 1.33 minutes MS m/z 489 [M + H]⁺ 3-Hydroxyazetidine |
| 36 | (S)-5-{[5-(6- aminopyridin-2-yl)-2- chlorobenzoyl]amino}-N- (1-hydroxypropan-2-yl)- 1-phenyl-1H-pyrazole-3- carboxamide | 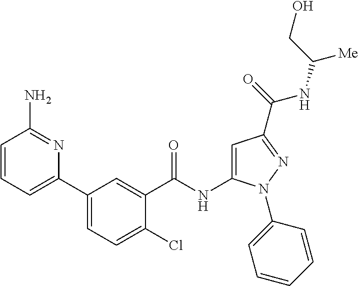 | LCMS Rt = 1.34 minutes MS m/z 491 [M + H]⁺ (S)-2-Aminopropan-1-ol |
| 37 | 5-{[5-(6-aminopyridin-2- yl)-2- chlorobenzoyl]amino}-N- [2-(1H-imidazol-2- yl)ethyl]-1-phenyl-1H- pyrazole-3-carboxamide | 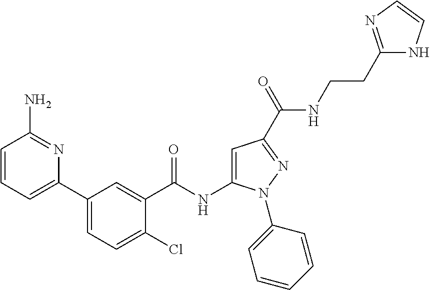 [SMILES: Nc1cccc(-c2ccc(Cl)c(C(=O)Nc3cc(C(=O)NCCc4ncc[nH]4)nn3-c3ccccc3)c2)n1] | LCMS Rt = 1.25 minutes MS m/z 527 [M + H]⁺ 2-(1H-imidazol-2- yl)ethan-1-amine |
| 38 | 5-{[5-(6-aminopyridin-2- yl)-2- chlorobenzoyl]amino}-N- [1- (hydroxymethyl)cyclo- butyl]-1-phenyl-1H- pyrazole-3-carboxamide | 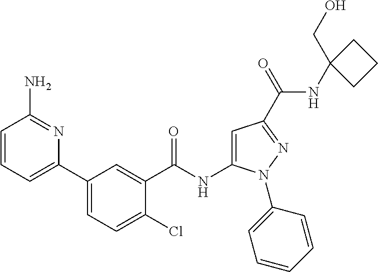 [SMILES: Nc1cccc(-c2ccc(Cl)c(C(=O)Nc3cc(C(=O)NC4(CO)CCC4)nn3-c3ccccc3)c2)n1] | LCMS Rt = 1.39 minutes MS m/z 517 [M + H]⁺ (1- Aminocyclobutyl)meth- anol |
| 39 | Racemic 5-{[5-(6- aminopyridin-2-yl)-2- chlorobenozyl]amino}-N- [(5-oxopyrrolidin-2- yl)methyl]-1-phenyl-1H- pyrazole-3-carboxamide | 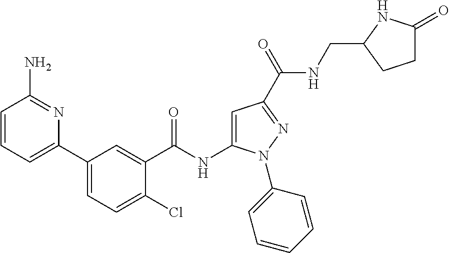 [SMILES: Nc1cccc(-c2ccc(Cl)c(C(=O)Nc3cc(C(=O)NCC4CCC(=O)N4)nn3-c3ccccc3)c2)n1] | LCMS Rt = 1.34 minutes MS m/z 530 [M + H]⁺ Racemic-5- (Aminomethyl)pyrrolidin- 2-one |
| 40 | 5-(6-aminopyridin-2-yl)- N-[3-(azetidin-1- ylcarbonyl)-1-phenyl-1H- pyrazol-5-yl]-2- chlorobenzamide | 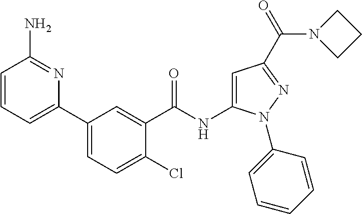 [SMILES: Nc1cccc(-c2ccc(Cl)c(C(=O)Nc3cc(C(=O)N4CCC4)nn3-c3ccccc3)c2)n1] | LCMS Rt = 1.41 minutes MS m/z 473 [M + H]⁺ Azetidine |
| 41 | 5-{[5-(6-aminopyridin-2- yl)-2- chlorobenzoyl]amino}-N- (3-methyl-1H-pyrazol-4- yl)-1-phenyl-1H- pyrazole-3-carboxamide | 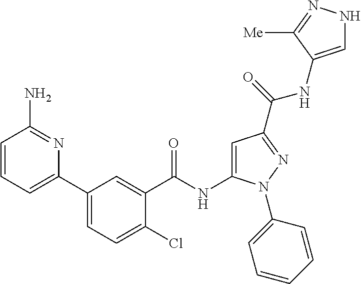 [SMILES: Cc1n[nH]cc1NC(=O)c1cc(NC(=O)c2cc(-c3cccc(N)n3)ccc2Cl)n(-c2ccccc2)n1] | LCMS Rt = 1.37 minutes MS m/z 513 [M + H]⁺ 3-Methyl-1H-pyrazol-4- amine |
| 42 | 5-{[5-(6-aminopyridin-2- yl)-2- chlorobenzoyl]amino}-N- [(3-methyl-1H-1,2,4- triazol-5-yl)methyl]-1- phenyl-1H-pyrazole-3- carboxamide | 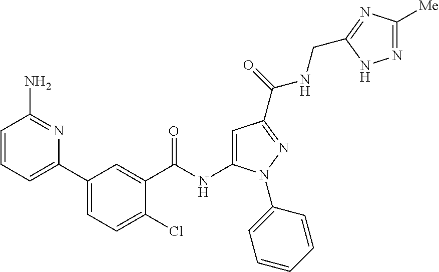 [SMILES: Cc1n[nH]c(CNC(=O)c2cc(NC(=O)c3cc(-c4cccc(N)n4)ccc3Cl)n(-c3ccccc3)n2)n1] | LCMS Rt = 1.33 minutes MS m/z 528 [M + H]⁺ (3-Methyl-1H-1,2,4- triazol-5-yl)methanamine |
| 43 | 5-{[5-(6-aminopyridin-2- yl)-2- chlorobenzoyl]amino}-N- [2-(dimethylamino)-2- oxoethyl]-1-phenyl-1H- pyrazole-3-carboxamide | 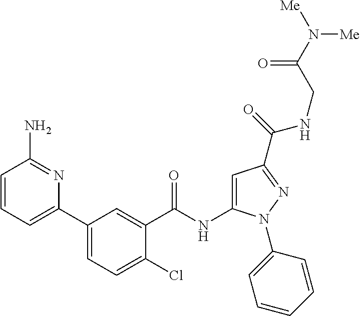 [SMILES: CN(C)C(=O)CNC(=O)c1cc(NC(=O)c2cc(-c3cccc(N)n3)ccc2Cl)n(-c2ccccc2)n1] | LCMS Rt = 1.36 minutes MS m/z 518 [M + H]⁺ 2-Amino-N,N- dimethylacetamide |
| 44 | (S)-5-(5-(6-amino-3,5- difluoropyridin-2-yl)-2- chlorobenzamido)-N-(1- hydroxypropan-2-yl)-1- phenyl-1H-pyrazole-3- carboxamide | 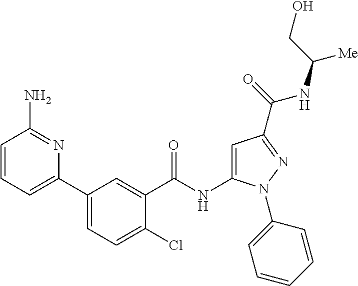 [SMILES: C[C@H](CO)NC(=O)c1cc(NC(=O)c2cc(-c3cccc(N)n3)ccc2Cl)n(-c2ccccc2)n1] | LCMS Rt = 1.34 minutes MS m/z 491 [M + H]⁺ (R)-2-Aminopropan-1-ol |
| 45 | 5-{[5-(6-aminopyridin-2- yl)-2- chlorobenzoyl]amino}-1- phenyl-N-(pyrimidin-2- ylmethyl)-1H-pyrazole-3- carboxamide | 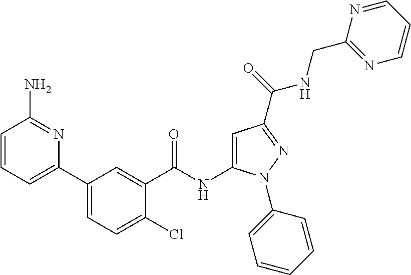 [SMILES: Nc1cccc(-c2ccc(Cl)c(C(=O)Nc3cc(C(=O)NCc4ncccn4)nn3-c3ccccc3)c2)n1] | LCMS Rt = 1.38 minutes MS m/z 525 [M + H]⁺ Pyrimidin-2- ylmethanamine |
| 46 | 5-{[5-(6-aminopyridin-2- yl)-2- chlorobenzoyl]amino}-N- [(3-methyl-1H-pyrazol-5- yl)methyl]-1-phenyl-1H- pyrazole-3-carboxamide | 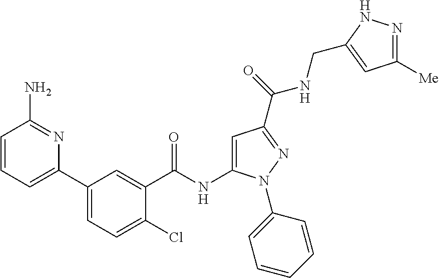 [SMILES: Cc1cc(CNC(=O)c2cc(NC(=O)c3cc(-c4cccc(N)n4)ccc3Cl)n(-c3ccccc3)n2)[nH]n1] | LCMS Rt = 1.38 minutes MS m/z 527 [M + H]⁺ (3-Methyl-1H-pyrazol-5- yl)methanamine |
| 47 | 5-{[5-(6-aminopyridin-2- yl)-2- chlorobenzoyl]amino}-N- (3-ethyloxetan-3-yl)-1- phenyl-1H-pyrazole-3- carboxamide | 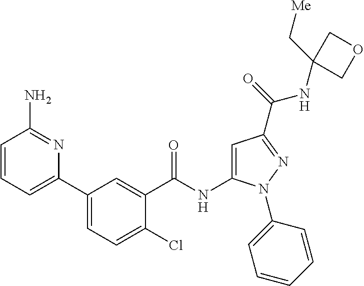 [SMILES: CCC1(NC(=O)c2cc(NC(=O)c3cc(-c4cccc(N)n4)ccc3Cl)n(-c3ccccc3)n2)COC1] | LCMS Rt = 1.40 minutes MS m/z 517 [M + H]⁺ 3-Ethyloxetan-3-amine |
| 48 | 5-{[5-(6-aminopyridin-2- yl)-2- chlorobenzoyl]amino}-N- cyclobutyl-1-phenyl-1H- pyrazole-3-carboxamide | 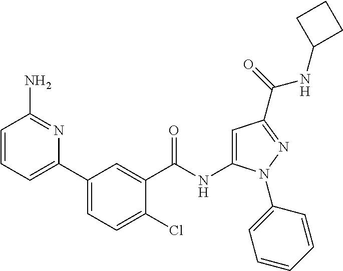 [SMILES: Nc1cccc(-c2ccc(Cl)c(C(=O)Nc3cc(C(=O)NC4CCC4)nn3-c3ccccc3)c2)n1] | LCMS Rt = 1.44 minutes MS m/z 487 [M + H]⁺ Cyclobutylamine |
| 49 | 5-{[5-(6-aminopyridin-2- yl)-2- chlorobenzoyl]amino}-N- (2,2-difluoropropyl)-1- phenyl-1H-pyrazole-3- carboxamide | 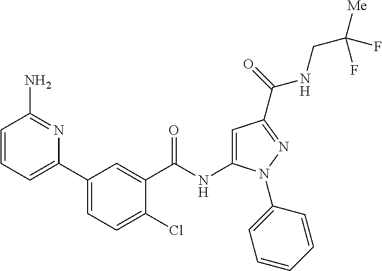 [SMILES: CC(F)(F)CNC(=O)c1cc(NC(=O)c2cc(-c3cccc(N)n3)ccc2Cl)n(-c2ccccc2)n1] | LCMS Rt = 1.43 minutes MS m/z 511 [M + H]⁺ 2,2-Difluoropropan-1- amine |
| 50 | Racemic-5-(6- aminopyridin-2-yl)-2- chloro-N-{3-[(3- fluoropyrrolidin-1- yl)carbonyl]-1-phenyl- 1H-pyrazol-5- yl)benzamide | 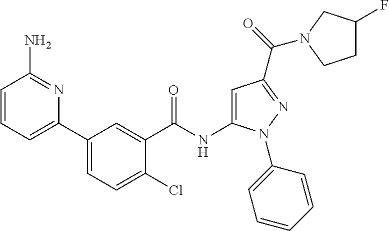 [SMILES: Nc1cccc(-c2ccc(Cl)c(C(=O)Nc3cc(C(=O)N4CCC(F)C4)nn3-c3ccccc3)c2)n1] | LCMS Rt = 1.42 minutes MS m/z 505 [M + H]⁺ Racemic-3- fluorocyclopentan-1- amine |
| 51 | 5-{5-(6-aminopyridin-2- yl)-2- chlorobenozyl]amino}-N- [3-(1H-imidazol-4- yl)propyl]-1-phenyl-1H- pyrazole-3-carboxamide | 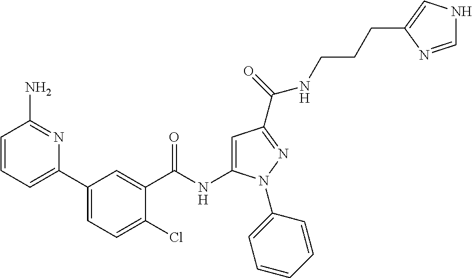 [SMILES: Nc1cccc(-c2ccc(Cl)c(C(=O)Nc3cc(C(=O)NCCCc4c[nH]cn4)nn3-c3ccccc3)c2)n1] | LCMS Rt = 1.27 minutes MS m/z 539 [M − H]⁻ 3-(1H-imidazol-4- yl)propan-1-amine |
| 52 | 5-{[5-(6-aminopyridin-2- yl)-2- chlorobenzoyl]amino}-1- phenyl-N-(1H-pyrazol-4- ylmethyl)-1H-pyrazole-3- carboxamide | 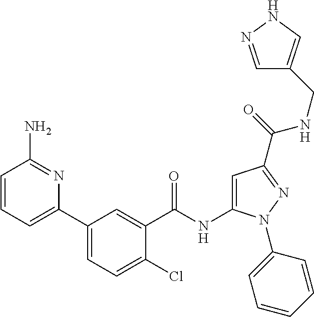 [SMILES: Nc1cccc(-c2ccc(Cl)c(C(=O)Nc3cc(C(=O)NCc4cn[nH]c4)nn3-c3ccccc3)c2)n1] | LCMS Rt = 1.35 minutes MS m/z 513 [M + H]⁺ (1H-Pyrazol-4- yl)methanamine |
| 53 | 5-{[5-(6-aminopyridin-2- yl)-2- chlorobenzoyl]amino}-1- phenyl-N-(1H-pyrazol-5- yl)-1H-pyrazole-3- carboxamide | 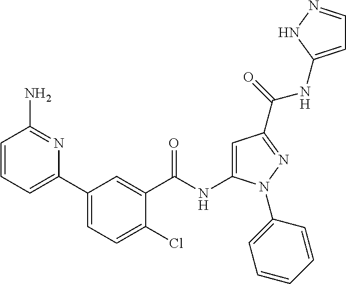 [SMILES: Nc1cccc(-c2ccc(Cl)c(C(=O)Nc3cc(C(=O)Nc4ccn[nH]4)nn3-c3ccccc3)c2)n1] | LCMS Rt = 1.37 minutes MS m/z 499 [M + H]⁺ 1H-Pyrazol-5-amine |
| 54 | 5-(6-aminopyridin-2-yl)- 2-chloro-N-{3-[(3- oxopiperazin-1- yl)carbonyl]-1-phenyl- 1H-pyrazol-5- yl}benzamide | 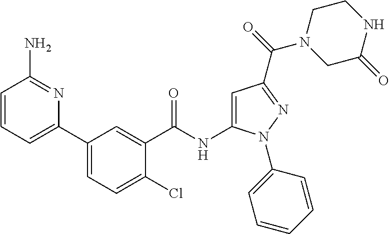 [SMILES: Nc1cccc(-c2ccc(Cl)c(C(=O)Nc3cc(C(=O)N4CCNC(=O)C4)nn3-c3ccccc3)c2)n1] | LCMS Rt = 1.32 minutes MS m/z 516 [M + H]⁺ Piperazin-2-one |
| 55 | 5-{[5-(6-aminopyridin-2- yl)-2- chlorobenzoyl]amino}-N- [(3-ethyloxetan-3- yl)methyl]-1-phenyl-1H- pyrazole-3-carboxamide | 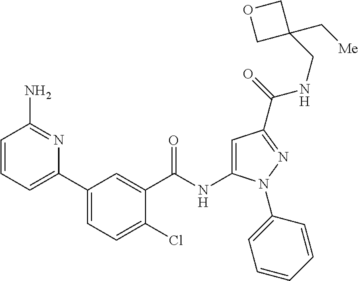 [SMILES: CCC1(CNC(=O)c2cc(NC(=O)c3cc(-c4cccc(N)n4)ccc3Cl)n(-c3ccccc3)n2)COC1] | LCMS Rt = 1.41 minutes MS m/z 531 [M + H]⁺ (3-Ethyloxetan-3- yl)methanamine |
| 56 | 5-{[5-(6-aminopyridin-2- yl)-2- chlorobenzoyl]amino}-1- phenyl-N-(pyridin-4- ylmethyl)-1H-pyrazole-3- carboxamide | 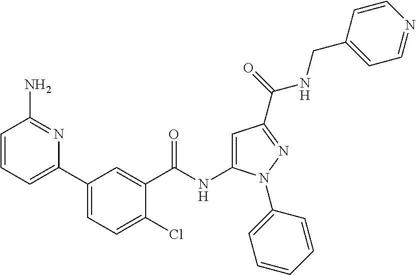 [SMILES: Nc1cccc(-c2ccc(Cl)c(C(=O)Nc3cc(C(=O)NCc4ccncc4)nn3-c3ccccc3)c2)n1] | LCMS Rt = 1.27 minutes MS m/z 524 [M + H]⁺ 4-Aminomethylpyridine |
| 57 | Racemic-5-{[5-(6- aminopyridin-2-yl)-2- chlorobenzoyl]amino}-1- phenyl-N-[1-(pyridin-2- yl)ethyl]-1H-pyrazole-3- carboxamide | 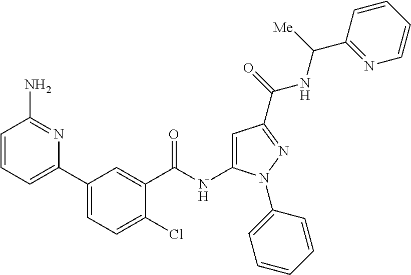 [SMILES: CC(NC(=O)c1cc(NC(=O)c2cc(-c3cccc(N)n3)ccc2Cl)n(-c2ccccc2)n1)c1ccccn1] | LCMS Rt = 1.37 minutes MS m/z 538 [M + H]⁺ Racemic-1-(pyridin-2- yl)ethan-1-amine |
| 58 | 5-{[5-(6-aminopyridin-2- yl)-2- chlorobenozyl]amino}-N- [(3-methyloxetan-3- yl)methyl]-1-phenyl-1H- pyrazole-3-carboxamide | 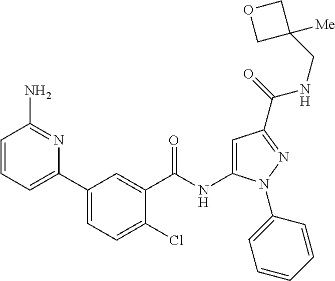 [SMILES: CC1(CNC(=O)c2cc(NC(=O)c3cc(-c4cccc(N)n4)ccc3Cl)n(-c3ccccc3)n2)COC1] | LCMS Rt = 1.38 minutes MS m/z 517 [M + H]⁺ (3-Methyloxetan-3- yl)methanamine |
| 59 | Racemic-1-[(5-{[5-(6- aminopyridin-2-yl)-2- chlorobenzoyl]amino}-1- phenyl-1H-pyrazol-3- yl)carbonyl]piperidine-3- carboxamide | 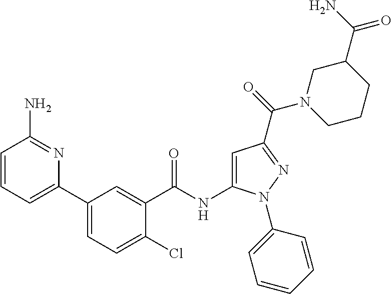 [SMILES: NC(=O)C1CCCN(C(=O)c2cc(NC(=O)c3cc(-c4cccc(N)n4)ccc3Cl)n(-c3ccccc3)n2)C1] | LCMS Rt = 1.34 minutes MS m/z 544 [M + H]⁺ Racemic-piperidine-3- carboxamide |
| 60 | 5-{[5-(6-aminopyridin-2- yl)-2- chlorobenozyl]amino}-N- [(1-methyl-1H-pyrazol-4- yl)methyl]-1-phenyl-1H pyrazole-3-carboxamide | 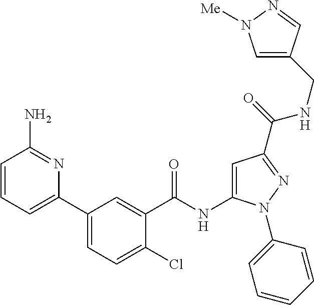 [SMILES: Cn1cc(CNC(=O)c2cc(NC(=O)c3cc(-c4cccc(N)n4)ccc3Cl)n(-c3ccccc3)n2)cn1] | LCMS Rt = 1.38 minutes MS m/z 527 [M + H]⁺ (1-Methyl-1H-pyrazol-4- yl)methanamine |
| 61 | 5-{[5-(6-aminopyridin-2- yl)-2- chlorobenozyl]amino}-N- (1,3-oxazol-4-ylmethyl)- 1-phenyl-1H-pyrazole-3- carboxamide | 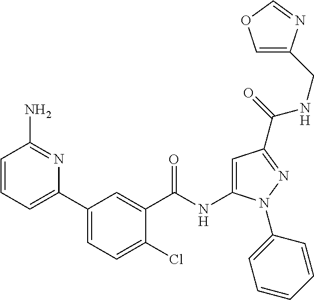 [SMILES: Nc1cccc(-c2ccc(Cl)c(C(=O)Nc3cc(C(=O)NCc4cocn4)nn3-c3ccccc3)c2)n1] | LCMS Rt = 1.37 minutes MS m/z 514 [M + H]⁺ Oxazol-4-ylmethanamine |
| 62 | 5-{[5-(6-aminopyridin-2- yl)-2- chlorobenzoyl]amino}-1- phenyl-N-(1H-1,2,4- triazol-3-yl)-1H-pyrazole- 3-carboxamide | 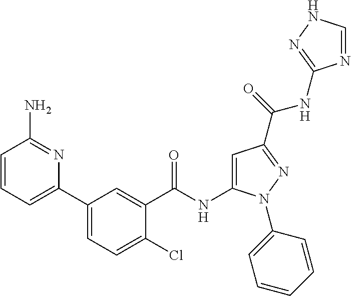 [SMILES: Nc1cccc(-c2ccc(Cl)c(C(=O)Nc3cc(C(=O)Nc4nc[nH]n4)nn3-c3ccccc3)c2)n1] | LCMS Rt = 1.34 minutes MS m/z 500 [M + H]⁺ 1H-1,2,4-Triazol-3-amine |
| 63 | 5-{[5-(6-aminopyridin-2- yl)-2- chlorobenzoyl]amino}-N- {[3- (hydroxymethyl)oxetan- 3-yl]methyl}-1-phenyl- 1H-pyrazole-3- carboxamide | 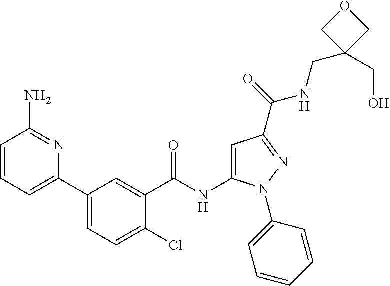 [SMILES: Nc1cccc(-c2ccc(Cl)c(C(=O)Nc3cc(C(=O)NCC4(CO)COC4)nn3-c3ccccc3)c2)n1] | LCMS Rt = 1.33 minutes MS m/z 533 [M + H]⁺ (3-(Aminomethyl)oxetan- 3-yl)methanol |
| 64 | 5-{[5-(6-aminopyridin-2- yl)-2- chlorobenzoyl]amino}-N- (3,3-difluorocyclobutyl)- 1-phenyl-1H-pyrazole-3- carboxamide | 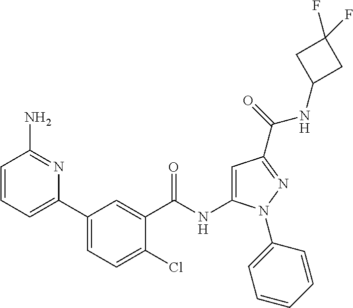 [SMILES: Nc1cccc(-c2ccc(Cl)c(C(=O)Nc3cc(C(=O)NC4CC(F)(F)C4)nn3-c3ccccc3)c2)n1] | LCMS Rt = 1.45 minutes MS m/z 523 [M + H]⁺ 3,3-Difluorocyclobutan-1- amine |
| 65 | 5-(6-aminopyridin-2-yl)- 2-chloro-N-{3-[(3- hydroxy-3- methylazetidin-1- yl)carbonyl]-1-phenyl- 1H-pyrazol-5- yl}benzamide | 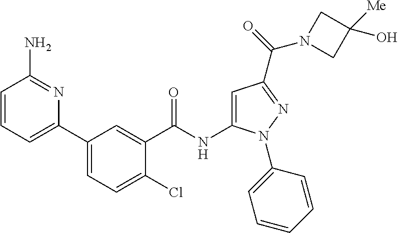 [SMILES: CC1(O)CN(C(=O)c2cc(NC(=O)c3cc(-c4cccc(N)n4)ccc3Cl)n(-c3ccccc3)n2)C1] | LCMS Rt = 1.35 minutes MS m/z 503 [M + H]⁺ 3-Methylazetidin-3-ol |
| 66 | 5-{[5-(6-aminopyridin-2- yl)-2- chlorobenzoyl]amino}-N- (1-methyl-1H-imidazol-2- yl)-1-phenyl-1H- pyrazole-3-carboxamide | 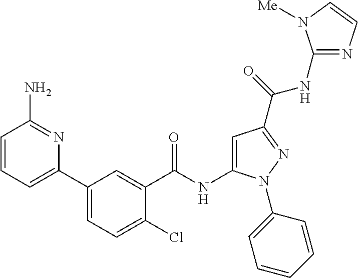 [SMILES: Cn1ccnc1NC(=O)c1cc(NC(=O)c2cc(-c3cccc(N)n3)ccc2Cl)n(-c2ccccc2)n1] | LCMS Rt = 1.28 minutes MS m/z 513 [M + H]⁺ 1-Methyl-1H-imidazol-2- amine |
| 67 | 5-{[5-(6-aminopyridin-2- yl)-2- chlorobenzoyl]amino}-1- phenyl-N-(4H-1,2,4- triazol-3-ylmethyl)-1H- pyrazole-3-carboxamide | 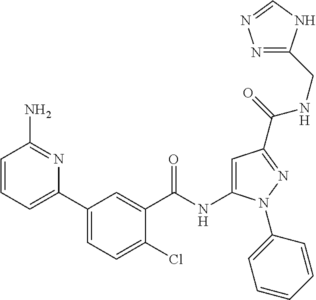 [SMILES: Nc1cccc(-c2ccc(Cl)c(C(=O)Nc3cc(C(=O)NCc4nnc[nH]4)nn3-c3ccccc3)c2)n1] | LCMS Rt = 1.32 minutes MS m/z 514 [M + H]⁺ (4H-1,2,4-Triazol-3- yl)methanamine |
| 68 | 5-(5-(6-amino-3,5- difluoropyridin-2-yl)-2- chlorobenzamido)-N- ((1R,3S)-3- hydroxycyclopentyl)-1- phenyl-1H-pyrazole-3- carboxamide | 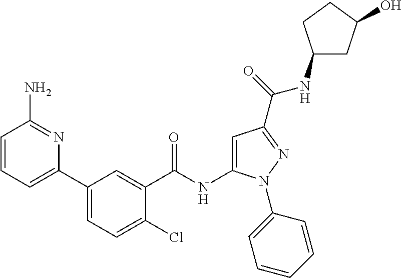 [SMILES: Nc1cccc(-c2ccc(Cl)c(C(=O)Nc3cc(C(=O)N[C@H]4CC[C@@H](O)C4)nn3-c3ccccc3)c2)n1] | LCMS Rt = 1.36 minutes MS m/z 517 [M + H]⁺ (1R,3S)-3- Aminocyclopentan-1-ol |
| 69 | Racemic 5-{[5-(6- aminopyridin-2-yl)-2- chlorobenzoyl]amino}-N- (1-methylpyrrolidin-3-yl)- 1-phenyl-1H-pyrazole-3- carboxamide | 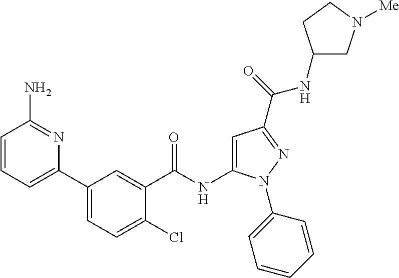 [SMILES: CN1CCC(NC(=O)c2cc(NC(=O)c3cc(-c4cccc(N)n4)ccc3Cl)n(-c3ccccc3)n2)C1] | LCMS Rt = 1.26 minutes MS m/z 516 [M + H]⁺ Racemic-1- methylpyrrolidin-3-amine |
| 70 | (S)-5-{[5-(6- aminopyridin-2-yl)-2- chlorobenzoyl]amino}-N- (2-hydroxypropyl)-1- phenyl-1H-pyrazole-3- carboxamide | 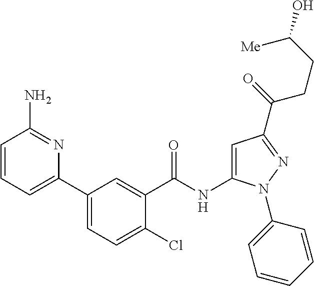 [SMILES: C[C@H](O)CCC(=O)c1cc(NC(=O)c2cc(-c3cccc(N)n3)ccc2Cl)n(-c2ccccc2)n1] | LCMS Rt = 1.33 minutes MS m/z 491 [M + H]⁺ (S)-1-Aminopropan-2-ol |
| 71 | 5-{[5-(6-aminopyridin-2- y)-2- chlorobenzoyl]amino}-N- (cyclopropylmethyl)-1- phenyl-1H-pyrazole-3- carboxamide | 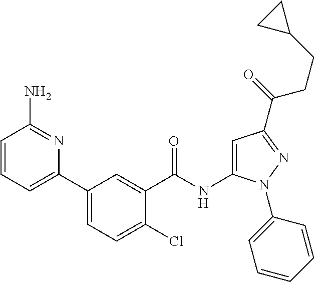 [SMILES: Nc1cccc(-c2ccc(Cl)c(C(=O)Nc3cc(C(=O)CCC4CC4)nn3-c3ccccc3)c2)n1] | LCMS Rt = 1.44 minutes MS m/z 487 [M + H]⁺ Cyclopropylmethylamine |
| 72 | 5-{[5-(6-aminopyridin-2- yl)-2- chlorobenozyl]amino}-1- phenyl-N-(pyridin-3- ylmethyl)-1H-pyrazole-3- carboxamide | 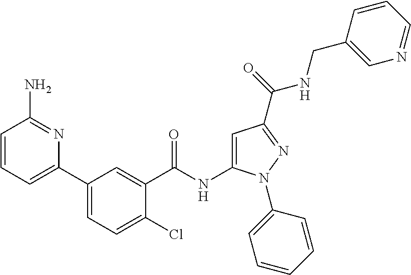 [SMILES: Nc1cccc(-c2ccc(Cl)c(C(=O)Nc3cc(C(=O)NCc4cccnc4)nn3-c3ccccc3)c2)n1] | LCMS Rt = 1.29 minutes MS m/z 524 [M + H]⁺ Pyridin-3-ylmethanamine |
| 73 | 5-{[5-(6-aminopyridin-2- yl)-2- chlorobenzoyl]amino}-N- [(1-methyl-1H-imidazol- 4-yl)methyl]-1-phenyl- 1H-pyrazole-3- carboxamide | 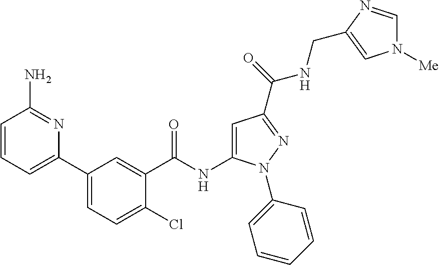 [SMILES: Cn1cnc(CNC(=O)c2cc(NC(=O)c3cc(-c4cccc(N)n4)ccc3Cl)n(-c3ccccc3)n2)c1] | LCMS Rt = 1.26 minutes MS m/z 527 [M + H]⁺ (1-Methyl-1H-imidazol-4- yl)methanamine |
| 74 | 5-{[5-(6-aminopyridin-2- yl)-2- chlorobenzoyl]amino}-N- (1,2-oxazol-3-ylmethyl)- 1-phenyl-1H-pyrazole-3- carboxamide | 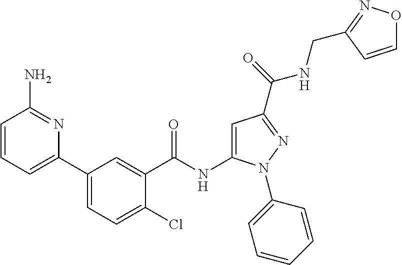 [SMILES: Nc1cccc(-c2ccc(Cl)c(C(=O)Nc3cc(C(=O)NCc4ccon4)nn3-c3ccccc3)c2)n1] | LCMS Rt = 1.39 minutes MS m/z 514 [M + H]⁺ isoxazol-3- ylmethanamine |
| 75 | 5-{[5-(6-aminopyridin-2- yl)-2- chlorobenzoyl]amino}-N- [2-(dimethylamino)ethyl]- 1-phenyl-1H-pyrazole-3- carboxamide | 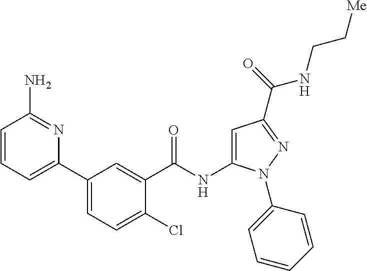 [SMILES: CCCNC(=O)c1cc(NC(=O)c2cc(-c3cccc(N)n3)ccc2Cl)n(-c2ccccc2)n1] | LCMS Rt = 1.25 minutes MS m/z 504 [M + H]⁺ N1,N1-Dimethylethane- 1,2-diamine |
| 76 | 5-{[5-(6-aminopyridin-2- yl)-2- chlorobenzoyl]amino}-1- phenyl-N-(1H-pyrazol-3- ylmethyl)-1H-pyrazole-3- carboxamide | 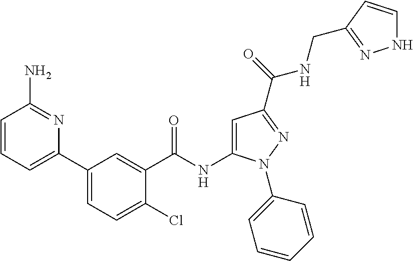 [SMILES: Nc1cccc(-c2ccc(Cl)c(C(=O)Nc3cc(C(=O)NCc4cc[nH]n4)nn3-c3ccccc3)c2)n1] | LCMS Rt = 1.36 minutes MS m/z 513 [M + H]⁺ (1H-Pyrazol-3- yl)methanamine |
| 77 | 5-{[5-(6-aminopyridin-2- yl)-2- chlorobenzoyl]amino}-N- [(6-methylpyridin-3- yl)methyl]-1-phenyl-1H- pyrazole-3-carboxamide | 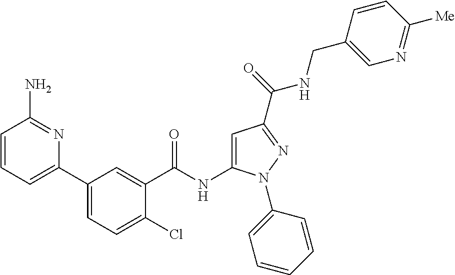 [SMILES: Cc1ccc(CNC(=O)c2cc(NC(=O)c3cc(-c4cccc(N)n4)ccc3Cl)n(-c3ccccc3)n2)cn1] | LCMS Rt = 1.28 minutes MS m/z 536 [M − H]⁻ (6-Methylpyridin-3- yl)methanamine |
| 78 | 5-{[5-(6-aminopyridin-2- yl)-2- chlorobenzoyl]amino}-1- phenyl-N-[2-(1H-1,2,3- triazol-1-yl)ethyl]-1H- pyrazole-3-carboxamide | 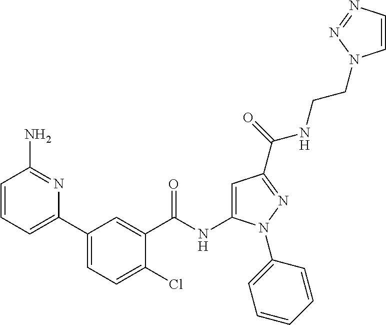 [SMILES: Nc1cccc(-c2ccc(Cl)c(C(=O)Nc3cc(C(=O)NCCn4ccnn4)nn3-c3ccccc3)c2)n1] | LCMS Rt = 1.35 minutes MS m/z 528 [M + H]⁺ 2-(1H-1,2,3-Triazol-1- yl)ethan-1-amine |
| 79 | Racemic 5-{[5-(6- aminopyridin-2-yl)-2- chlorobenzyl]amino}-1- phenyl-N- (tetrahydrofuran-3- ylmethyl)-1H-pyrazole-3- carboxamide | 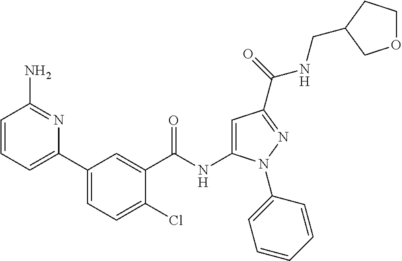 [SMILES: Nc1cccc(-c2ccc(Cl)c(C(=O)Nc3cc(C(=O)NCC4CCOC4)nn3-c3ccccc3)c2)n1] | LCMS Rt = 1.38 minutes MS m/z 417 [M + H]⁺ (Tetrahydrofuran-3- yl)methanamine |
| 80 | (S)-N-(1-acetylpyrrolidin- 3-yl)-5-{[5-(6- aminopyridin-2-yl)-2- chlorobenzoyl]amino}-1- phenyl-1H-pyrazole-3- carboxamide | 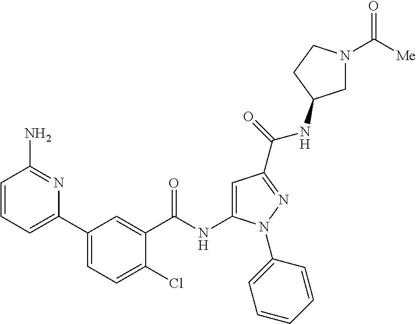 [SMILES: CC(=O)N1CC[C@H](NC(=O)c2cc(NC(=O)c3cc(-c4cccc(N)n4)ccc3Cl)n(-c3ccccc3)n2)C1] | LCMS Rt = 1.36 minutes MS m/z 544 [M + H]⁺ (S)-1-(3-Aminopyrrolidin- 1-yl)ethan-1-one |
| 81 | 5-{[5-(6-aminopyridin-2- yl)-2- chlorobenozyl]amino}-N- [(1-methyl-1H-imidazol- 2-yl)methyl]-1-phenyl- 1H-pyrazole-3- carboxamide | 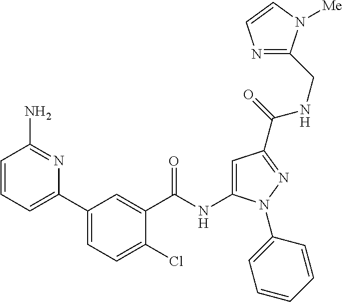 [SMILES: Cn1ccnc1CNC(=O)c1cc(NC(=O)c2cc(-c3cccc(N)n3)ccc2Cl)n(-c2ccccc2)n1] | LCMS Rt = 1.26 minutes MS m/z 527 [M + H]⁺ (1-Methyl-1H-imidazol-2- yl)methanamine |
| 82 | 5-{[5-(6-aminopyridin-2- yl)-2- chlorobenzoyl]amino}-1- phenyl-N-(pyrazin-2- ylmethyl)-1H-pyrazole-3- carboxamide | 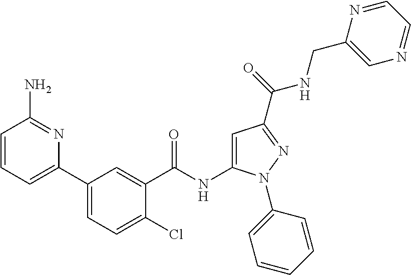 [SMILES: Nc1cccc(-c2ccc(Cl)c(C(=O)Nc3cc(C(=O)NCc4cnccn4)nn3-c3ccccc3)c2)n1] | LCMS Rt = 1.37 minutes MS m/z 525 [M + H]⁺ Pyrazin-2-ylmethanamine |
| 83 | 5-{[5-(6-aminopyridin-2- yl)-2- chlorobenzoyl]amino}-1- phenyl-N-(pyrimidin-5- yl)-1H-pyrazole-3- carboxamide | 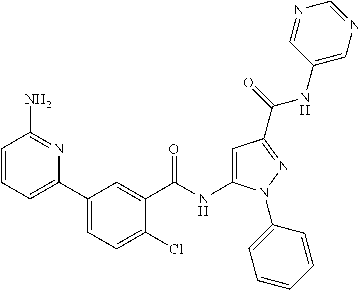 [SMILES: Nc1cccc(-c2ccc(Cl)c(C(=O)Nc3cc(C(=O)Nc4cncnc4)nn3-c3ccccc3)c2)n1] | LCMS Rt = 1.40 minutes MS m/z 511 [M + H]⁺ Pyrimidin-5-amine |
| 84 | Racemic-5-(6- aminopyridin-2-yl)-2- chloro-N-{3-[(3-hydroxy- 3-methylpyrrolidin-1- yl)carbonyl]-1-phenyl- 1H-pyrazol-5- yl}benzamide | 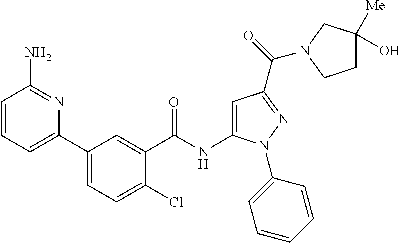 [SMILES: CC1(O)CCN(C(=O)c2cc(NC(=O)c3cc(-c4cccc(N)n4)ccc3Cl)n(-c3ccccc3)n2)C1] | LCMS Rt = 1.36 minutes MS m/z 517 [M + H]⁺ Racemic-3- methylpyrrolidin-3-ol |
| 85 | (S)-5-{[5-(6- aminopyridin-2-yl)-2- chlorobenozyl]amino}-N- (5-oxopyrrolidin-3-yl)-1- phenyl-1H-pyrazole-3- carboxamide | 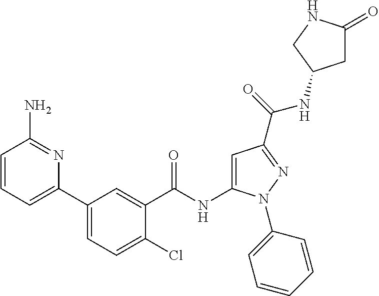 [SMILES: Nc1cccc(-c2ccc(Cl)c(C(=O)Nc3cc(C(=O)N[C@@H]4CNC(=O)C4)nn3-c3ccccc3)c2)n1] | LCMS Rt = 1.32 minutes MS m/z 516 [M + H]⁺ (S)-4-Aminopyrrolidin-2- one |
| 86 | 5-{[5-(6-aminopyridin-2- yl)-2- chlorobenzyl]amino}-1- phenyl-N-(pyridin-2- ylmethyl)-1H-pyrazole-3- carboxamide | 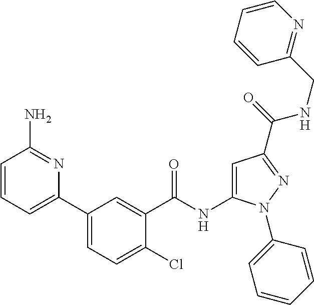 [SMILES: Nc1cccc(-c2ccc(Cl)c(C(=O)Nc3cc(C(=O)NCc4ccccn4)nn3-c3ccccc3)c2)n1] | LCMS Rt = 1.37 minutes MS m/z 524 [M + H]⁺ Pyridin-2-ylmethanamine |
| 87 | 5-{[5-(6-aminopyridin-2- yl)-2- chlorobenzoyl]amino}-1- phenyl-N-(1H-1,2,3- triazol-4-ylmethyl)-1H- pyrazole-3-carboxamide | 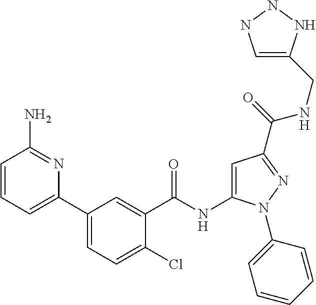 [SMILES: Nc1cccc(-c2ccc(Cl)c(C(=O)Nc3cc(C(=O)NCC4=CNNN4)nn3-c3ccccc3)c2)n1] | LCMS Rt = 1.33 minutes MS m/z 514 [M + H]⁺ (1H-1,2,3-Triazol-5- yl)methanamine |
| 88 | (S)-1-[(5-{[5-(6- aminopyridin-2-yl)-2- chlorobenozyl]amino}-1- phenyl-1H-pyrazol-3- yl)carbonyl]piperidine-3- carboxamide | 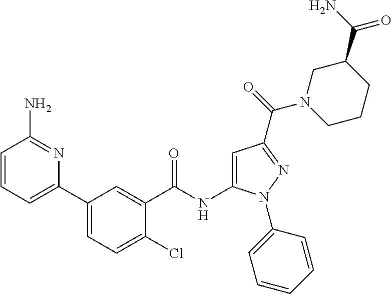 [SMILES: NC(=O)[C@H]1CCCN(C(=O)c2cc(NC(=O)c3cc(-c4cccc(N)n4)ccc3Cl)n(-c3ccccc3)n2)C1] | LCMS Rt = 1.34 minutes MS m/z 544 [M + H]⁺ (S)-Piperidine-3- carboxamide |
| 89 | 5-{[5-(6-aminopyridin-2- yl)-2- chlorobenzoyl]amino}-N- (1-methyl-1H-imidazol-4- yl)-1-phenyl-1H- pyrazole-3-carboxamide | 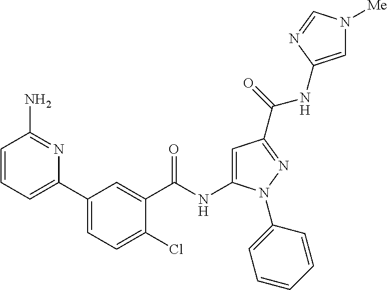 [SMILES: Cn1cnc(NC(=O)c2cc(NC(=O)c3cc(-c4cccc(N)n4)ccc3Cl)n(-c3ccccc3)n2)c1] | LCMS Rt = 1.32 minutes MS m/z 513 [M + H]⁺ 1-methyl-1H-imidazol-4- amine |
| 90 | 5-{[5-(6-aminopyridin-2- yl)-2- chlorobenzyl]amino}-N- (1,3-oxazol-2-ylmethyl)- 1-phenyl-1H-pyrazole-3- carboxamide | 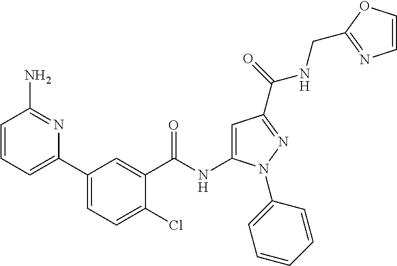 [SMILES: Nc1cccc(-c2ccc(Cl)c(C(=O)Nc3cc(C(=O)NCc4ncco4)nn3-c3ccccc3)c2)n1] | LCMS Rt = 1.37 minutes MS m/z 514 [M + H]⁺ Oxazol-2-ylmethanamine |
| 91 | 5-{[5-(6-aminopyridin-2- yl)-2- chlorobenzoyl]amino}-N- [(1-methyl-1H-imidazol- 5-yl)methyl]-1-phenyl- 1H-pyrazole-3- carboxamide | 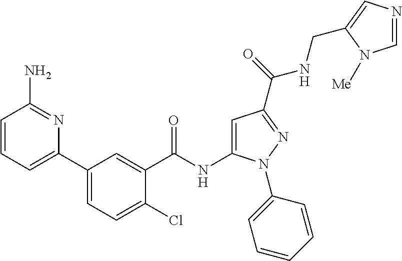 [SMILES: Cn1cncc1CNC(=O)c1cc(NC(=O)c2cc(-c3cccc(N)n3)ccc2Cl)n(-c2ccccc2)n1] | LCMS Rt = 1.25 minutes MS m/z 525 [M − H]⁻ (1-Methyl-1H-imidazol-5- yl)methanamine |
| 92 | 5-{[5-(6-aminopyridin-2- yl)-2- chlorobenozyl]amino}-N- [2-(oxetan-3-yl)ethyl]-1- phenyl-1H-pyrazole-3- carboxamide | 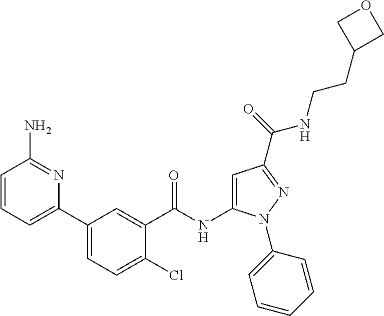 [SMILES: Nc1cccc(-c2ccc(Cl)c(C(=O)Nc3cc(C(=O)NCCC4COC4)nn3-c3ccccc3)c2)n1] | LCMS Rt = 1.38 minutes MS m/z 517 [M + H]⁺ 2-(Oxetan-3-yl)ethan-1- amine |
| 93 | 5-{[5-(6-aminopyridin-2- yl)-2- chlorobenzoyl]amino}-N- (1-methyl-1H-pyrazol-4- yl)-1-phenyl-1H- pyrazole-3-carboxamide | 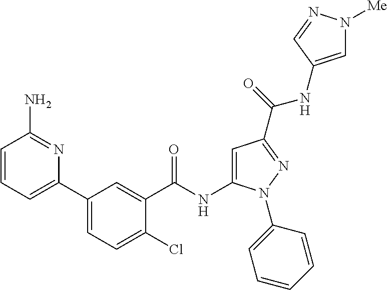 [SMILES: Cn1cc(NC(=O)c2cc(NC(=O)c3cc(-c4cccc(N)n4)ccc3Cl)n(-c3ccccc3)n2)cn1] | LCMS Rt = 1.40 minutes MS m/z 513 [M + H]⁺ 1-Methyl-1H-pyrazol-4- amine |
| 94 | 5-{[5-(6-aminopyridin-2- yl)-2- chlorobenzoyl]amino}-N- (1,2-oxazol-5-ylmethyl)- 1-phenyl-1H-pyrazole-3- carboxamide | 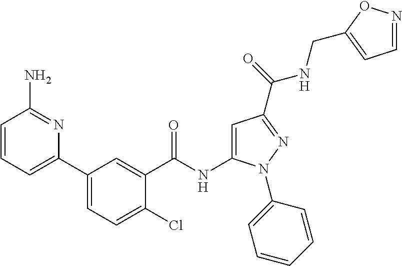 [SMILES: Nc1cccc(-c2ccc(Cl)c(C(=O)Nc3cc(C(=O)NCc4ccno4)nn3-c3ccccc3)c2)n1] | LCMS Rt = 1.38 minutes MS m/z 514 [M + H]⁺ Isoxazol-5- ylmethanamine |
| 95 | 5-{[5-(6-aminopyridin-2- yl)-2- chlorobenzoyl]amino}-N- [(1- hydroxycyclobutyl)methyl]- 1-phenyl-1H-pyrazole- 3-carboxamide | 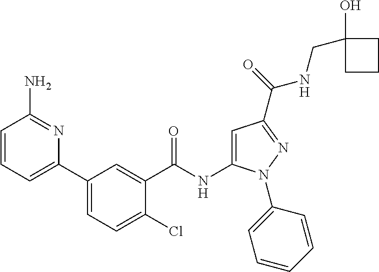 [SMILES: Nc1cccc(-c2ccc(Cl)c(C(=O)Nc3cc(C(=O)NCC4(O)CCC4)nn3-c3ccccc3)c2)n1] | LCMS Rt = 1.38 minutes MS m/z 517 [M + H]⁺ 1- (Aminomethyl)cyclobutan- 1-ol |
| 96 | 5-{[5-(6-aminopyridin-2- yl)-2- chlorobenzoyl]amino}-1- phenyl-N-[2-(1H-pyrazol- 4-yl)ethyl]-1H-pyrazole- 3-carboxamide | 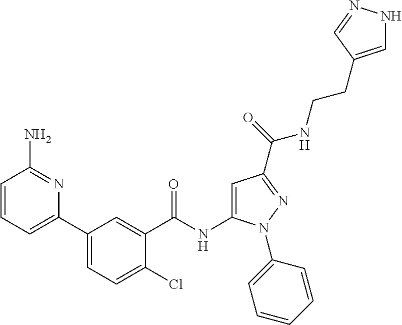 [SMILES: Nc1cccc(-c2ccc(Cl)c(C(=O)Nc3cc(C(=O)NCCc4cn[nH]c4)nn3-c3ccccc3)c2)n1] | LCMS Rt = 1.37 minutes MS m/z 527 [M + H]⁺ 2-(1H-Pyrazol-4-yl)ethan- 1-amine |
| 97 | 5-{[5-(6-aminopyridin-2- yl)-2- chlorobenzoyl]amino}-N- (1,3-oxazol-5-ylmethyl)- 1-phenyl-1H-pyrazole-3- carboxamide | 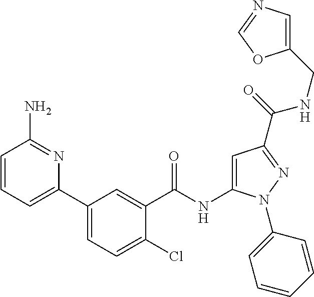 [SMILES: Nc1cccc(-c2ccc(Cl)c(C(=O)Nc3cc(C(=O)NCc4cnco4)nn3-c3ccccc3)c2)n1] | LCMS Rt = 1.37 minutes MS m/z 514 [M + H]⁺ Oxazol-5-ylmethanamine |
| 98 | (1S,2R)-5-{[5-(6- aminopyridin-2-yl)-2- chlorobenzoyl]amino}-N- (2-hydroxycyclopentyl)- 1-phenyl-1H-pyrazole-3- carboxamide | 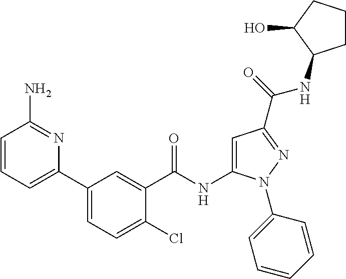 [SMILES: Nc1cccc(-c2ccc(Cl)c(C(=O)Nc3cc(C(=O)N[C@@H]4CCC[C@@H]4O)nn3-c3ccccc3)c2)n1] | LCMS Rt = 1.38 minutes MS m/z 517 [M + H]⁺ (1S,2R)-2- Aminocyclopentan-1-ol |
| 99 | 5-{[5-(6-aminopyridin-2- yl)-2- chlorobenzoyl]amino}-N- [2-(1H-imidazol-4- yl)ethyl]-1-phenyl-1H- pyrazole-3-carboxamide | 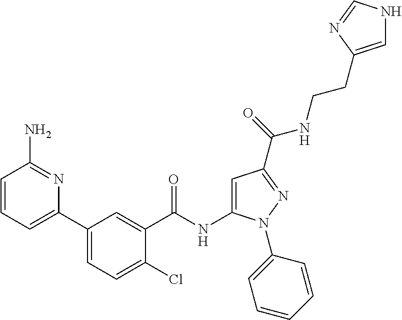 [SMILES: Nc1cccc(-c2ccc(Cl)c(C(=O)Nc3cc(C(=O)NCCc4c[nH]cn4)nn3-c3ccccc3)c2)n1] | LCMS Rt = 1.25 minutes MS m/z 527 [M + H]⁺ 2-(1H-imidazol-4- yl)ethan-1-amine |
| 100 | N-[2-(acetylamino)ethyl]- 5-{[5-(6-aminopyridin-2- yl)-2- chlorobenzoyl]amino}-1- phenyl-1H-pyrazole-3- carboxamide | 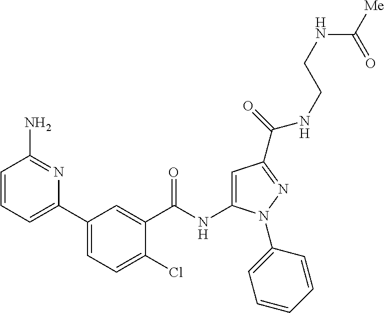 [SMILES: CC(=O)NCCNC(=O)c1cc(NC(=O)c2cc(-c3cccc(N)n3)ccc2Cl)n(-c2ccccc2)n1] | LCMS Rt = 1.33 minutes MS m/z 518 [M + H]⁺ N-(2- Aminoethyl)acetamide |

### Example 101

### 5-(5-(6-amino-3,5-difluoropyridin-2-yl)-2-chlorobenzamido)-N-((1r,4r)-4-hydroxy-4-methylcyclohexyl)-1-phenyl-1H-pyrazole-3-carboxamide

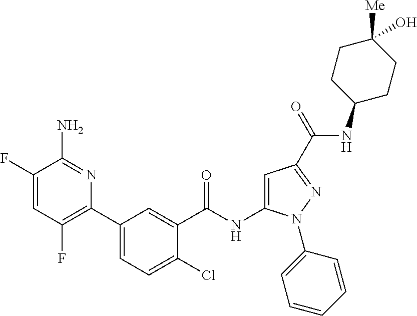 [SMILES: C[C@]1(O)CC[C@H](NC(=O)c2cc(NC(=O)c3cc(-c4nc(N)c(F)cc4F)ccc3Cl)n(-c3ccccc3)n2)CC1]

To a solution of 5-(5-(6-amino-3,5-difluoropyridin-2-yl)-2-chlorobenzamido)-1-phenyl-1H-pyrazole-3-carboxylic acid (Example 139, 80 mg, 0.170 mmol), (1r,4r)-4-amino-1-methylcyclohexanol hydrochloride salt (42 mg, 0.255 mmol) and DIPEA (148 μL, 0.851 mmol) in DMF (1 mL) was added HATU (97 mg, 0.255 mmol) and the reaction was stirred at room temperature for 1 hour. Saturated aqueous sodium hydrogen carbonate solution (30 mL) was added and the aqueous extracted with DCM (3×20 mL). The combined organic layers were washed with saturated aqueous sodium hydrogen carbonate solution (30 mL), filtered through a phase separation cartridge and concentrated in vacuo. The residue was purified by elution through an SCX-2 cartridge using MeOH followed by 7N NH₃ in MeOH followed by trituration with DCM to afford the title compound (41 mg, 41%).

¹H NMR (400 MHz, DMSO-d₆): δ ppm 1.13 (s, 3H), 1.38-1.56 (m, 6H), 1.67-1.71 (m, 2H), 3.77-3.79 (m, 1H), 4.26 (s, 1H), 6.38 (br s, 2H), 6.89 (s, 1H), 7.46-7.63 (m, 6H), 7.73 (t, 1H), 7.89 (br s, 2H), 7.91-7.95 (m, 1H), 10.77 (br s, 1H).

LCMS Rt=2.67 minutes MS m/z 581 [M+H]⁺

### Example 102

### 5-(5-(6-aminopyridin-2-yl)-2-chlorobenzamido)-N-methyl-1-phenyl-1H-pyrazole-3-carboxamide

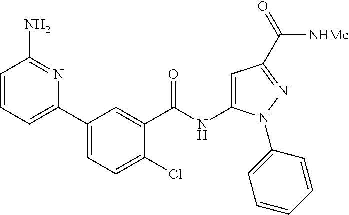 [SMILES: CNC(=O)c1cc(NC(=O)c2cc(-c3cccc(N)n3)ccc2Cl)n(-c2ccccc2)n1]

To a solution of 5-(5-(6-((tert-butoxycarbonyl)amino)pyridin-2-yl)-2-chlorobenzamido)-1-phenyl-1H-pyrazole-3-carboxylic acid (Preparation 1, 100 mg, 0.19 mmol) in DCM (2 mL) was added EDCI (43 mg, 0.24 mmol), HOBt (33 mg, 0.24 mmol) and methylamine (2M in THF, 93 mL, 0.19 mmol). The reaction was stirred at room temperature for 18 hours before concentrating in vacuo. The residue was purified using reverse phase column chromatography and suspended in DCM (3 mL). TFA (1 mL) was added and the reaction stirred at room temperature for 6 hours. The reaction was concentrated in vacuo and eluted through an SCX-2 column using MeOH followed by 7N NH₃ in MeOH to afford the title compound.

¹H NMR (400 MHz, DMSO-d₆): δ ppm 2.78 (d, 3H), 6.09 (br s, 2H), 6.47 (d, 1H), 6.90 (s, 1H), 7.11 (d, 1H), 7.46-7.67 (m, 7H), 8.06-8.09 (m, 2H), 8.28 (d, 1H), 10.78 (br s, 1H).

MS m/z 447 [M+H]⁺

### Example 103

### 5-(5-(6-aminopyridin-2-yl)-2-chlorobenzamido)-N-(2-methoxyethyl)-1-phenyl-1H-pyrazole-3-carboxamide

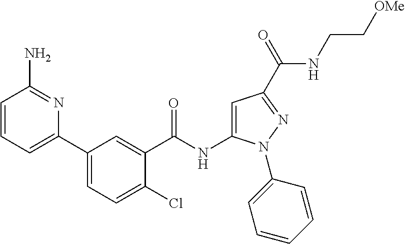 [SMILES: COCCNC(=O)c1cc(NC(=O)c2cc(-c3cccc(N)n3)ccc2Cl)n(-c2ccccc2)n1]

A solution of 5-(5-(6-((tert-butoxycarbonyl)amino)pyridin-2-yl)-2-chlorobenzamido)-1-phenyl-1H-pyrazole-3-carboxylic acid (Preparation 1, 76 mg, 0.142 mmol), 1-hydroxybenzotriazole hydrate (23 mg, 0.149 mmol), 2-methoxyethan-1-amine (16 mg, 0.213 mmol) and triethylamine (0.0590 mL, 0.424 mmol) in DMF (2 mL) was treated with 1-ethyl-3-(3-dimethylaminopropyl) carbodiimide (33 mg, 0.171 mmol) and stirred for 16 hours. Further 2-methoxyethan-1-amine (16 mg, 0.213 mmol), triethylamine (0.0390 mL, 0.284 mmol) and 1-ethyl-3-(3-dimethylaminopropyl) carbodiimide (13.6 mg, 0.071 mmol) was added and the reaction stirred for 23 hours. The reaction was diluted with ethyl acetate (10 mL) and washed with saturated aqueous NaHCO₃ solution (2×10 mL) followed by 10% aqueous citric acid solution (10 mL). The organic layer was collected, dried over sodium sulphate and concentrated in vacuo. The residue was purified using silica gel column chromatography eluting with 50-90% EtOAc in heptanes. The residue was dissolved in dichloromethane (0.3 mL), treated with trifluoroacetic acid (0.3 mL) and stirred at room temperature for 2 hours. The reaction was eluted through an SCX-2 column using MeOH followed by 2N NH₃ in MeOH. The resulting solution was concentrated in vacuo and purified using silica gel column chromatography eluting with 70% EtOAc in heptanes to afford the title compound.

¹H NMR (400 MHz, DMSO-d₆): δ ppm 2.26 (s, 3H), 3.38-3.48 (m, 4H), 6.08 (s, 2H), 6.47 (d, 1H), 6.90 (s, 1H), 7.10 (d, 1H), 7.45-7.63 (m, 6H), 8.03-8.12 (m, 2H), 8.22 (t, 1H), 10.77 (s, 1H).

LCMS Rt=2.63 minutes MS m/z 491 [M+H]⁺

The following Examples were prepared according to the Method described by Example 103 using 5-(5-(6-((tert-butoxycarbonyl)amino)pyridin-2-yl)-2-chlorobenzamido)-1-phenyl-1H-pyrazole-3-carboxylic acid (Preparation 1) and the appropriate amine as described below. The crude residues were purified as above or according to the purification method described below:

Purification Method (PM) A: Silica gel column chromatography eluting with 60% EtOAc in heptanes.
Purification Method B: Elution through an SCX-2 column using methanol followed by 7N NH₃ in MeOH.
Purification Method C: Reverse phase column chromatography.

| Example | | | |
|---|---|---|---|
| Number | Name | Structure | Data/Amine/PM |
| 104 | 5-(5-(6-aminopyridin-2- yl)-2-chlorobenzamido)- N-((1s,4s)-4- hydroxycyclohexyl)-1- phenyl-1H-pyrazole-3- carboxamide | 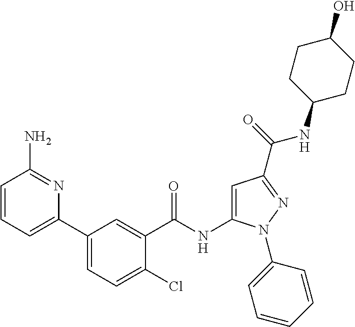 [SMILES: Nc1cccc(-c2ccc(Cl)c(C(=O)Nc3cc(C(=O)N[C@H]4CC[C@@H](O)CC4)nn3-c3ccccc3)c2)n1] | ¹H NMR (400 MHz, DMSO- d₆): δ ppm 1.43-1.82 (s, 8H), 3.71-3.85 (m, 2H), 4.34 (s, 1H), 6.06 (br s, 2H), 6.93 (s, 1H), 7.09 (d, 1H), 7.40-7.60 (s, 6H), 7.92 (d, 1H), 8.03-8.09 (m, 2H), 10.77 (s, 1H). LCMS Rt = 2.49 minutes MS m/z 531 [M + H]⁺ (1s,4s)-4-Aminocyclohexan- 1-ol. PM A. |
| 105 | Racemic-5-(5-(6- aminopyridin-2-yl)-2- chlorobenzamido)-N-(2- hydroxypropyl)-1-phenyl- 1H-pyrazole-3- carboxamide | 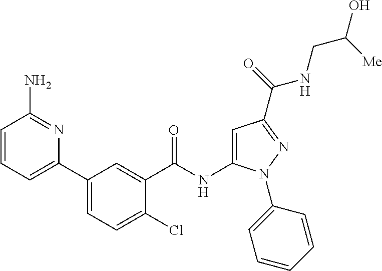 [SMILES: CC(O)CNC(=O)c1cc(NC(=O)c2cc(-c3cccc(N)n3)ccc2Cl)n(-c2ccccc2)n1] | ¹H NMR (400 MHz, MeOH- d₄): δ ppm 3.30-3.37 (m, 3H), 3.45-3.49 (dd, 1H), 3.92-4.00 (m, 1H), 6.54 (d, 1H), 6.89 (s, 1H), 7.03 (d, 1H), 7.37-7.40 (m, 1H), 7.43-7.53 (m, 5H), 7.76 (d, 1H), 7.85 (d, 1H), 8.00 (d, 1H). MS m/z 491 [M + H]⁺ Racemic-1-amino-2- propanol. PM B. |
| 106 | Cis + trans 5-(5-(6- aminopyridin-2-yl)-2- chlorobenzamido)-N-(3- hydroxycyclobutyl)-1- phenyl-1H-pyrazole-3- carboxamide | 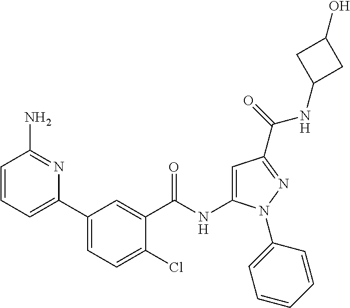 [SMILES: Nc1cccc(-c2ccc(Cl)c(C(=O)Nc3cc(C(=O)NC4CC(O)C4)nn3-c3ccccc3)c2)n1] | ¹H NMR (400 MHz, DMSO- d₆): δ ppm 1.95 (m, 2H), 2.13 (m, 1H), 2.32 (m, 1H), 3.85-4.40 (m, 2H), 5.05 (dd, 1H), 6.10 (s, 2H), 6.46 (d, 1H), 6.92 (s, 1H), 7.10 (d, 1H), 7.48-7.52 (m, 5H), 7.57 (m, 2H), 8.07 (d, 2H), 8.41 (dd, 1H), 10.78 (s, 1H). MS m/z 503 [M + H]⁺ 3-Aminocyclobutanol hydrochloride. PM C. |

### Example 107

### Racemic-5-(5-(6-aminopyridin-2-yl)-2-chlorobenzamido)-N-(1-hydroxy-3-methoxypropan-2-yl)-1-phenyl-1H-pyrazole-3-carboxamide

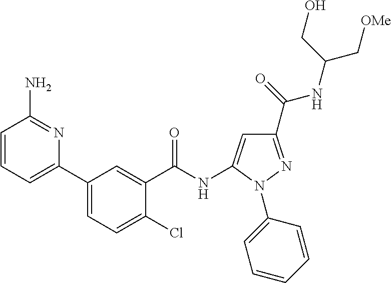 [SMILES: COCC(CO)NC(=O)c1cc(NC(=O)c2cc(-c3cccc(N)n3)ccc2Cl)n(-c2ccccc2)n1]

The title compound was prepared according to the method described for Example 1 using 5-(5-(6-((tert-butoxycarbonyl)amino)pyridin-2-yl)-2-chlorobenzamido)-1-phenyl-1H-pyrazole-3-carboxylic acid (Preparation 1) and racemic-2-amino-3-methoxypropan-1-ol with triethylamine. The residue was purified using silica gel column chromatography eluting with EtOAc, dissolved in DCM and treated with TFA with stirring for 18 hours. The reaction was concentrated in vacuo and dissolved in MeOH. Saturated aqueous NaHCO₃ solution was added with stirring for 1 hour. The solution was concentrated in vacuo and purified using silica gel column chromatography eluting with EtOAc to afford the title compound.

¹H NMR (400 MHz, MeOH-d₄): δ ppm 3.37-3.42 (m, 3H), 3.53-3.65 (m, 2H), 3.65-3.77 (m, 2H), 4.23-4.32 (m, 1H), 6.54-6.59 (d, 1H), 7.02-7.07 (m, 2H), 7.47-7.60 (m, 5H), 7.60-7.67 (m, 2H), 7.92-8.02 (m, 2H).

LCMS Rt=2.49 minutes MS m/z 521 [M+H]⁺

The following Examples were prepared according to the Method described by Example 103 using 5-(5-(6-((tert-butoxycarbonyl)amino)pyridin-2-yl)-2-chlorobenzamido)-1-phenyl-1H-pyrazole-3-carboxylic acid (Preparation 1) and the appropriate amine as described below. The crude residues were purified as above or according to the purification method described below:

Purification Method A: Trituration with TBME.
Purification Method B: Elution through an SCX-2 column using MeOH followed by 7N NH₃ in MeOH.
Purification Method C: The residue was treated with 7N ammonia in MeOH (2 mL), concentrated in vacuo and purified by silica gel column chromatography eluting with 40% heptane/acetone followed by elution through an SCX-2 column using MeOH followed by 7N NH₃ in MeOH.

| Example | | | |
|---|---|---|---|
| Number | Name | Structure | Data/Amine/PM |
| 108 | Racemic-5-(5-(6- aminopyridin-2-yl)-2- chlorobenzamido)-N- (2,3-dihydroxypropyl)-1- phenyl-1H-pyrazole-3- carboxamide | 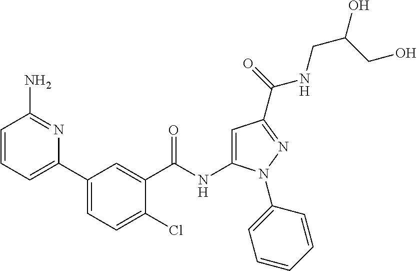 [SMILES: Nc1cccc(-c2ccc(Cl)c(C(=O)Nc3cc(C(=O)NCC(O)CO)nn3-c3ccccc3)c2)n1] | ¹H NMR (400 MHz, MeOH- d₄): δ ppm 3.25-3.40 (m, 2H), 3.55 (m, 2H), 3.80 (m, 1H), 6.60 (m, 1H), 7.00 (m, 2H), 7.45-7.70 (m, 6H), 8.00 (m, 2H), 8.25 (m, 1H). LCMS Rt = 2.53 minutes MS m/z 507 [M + H]⁺ Racemic-3-aminopropane- 1,2-diol |
| 109 | 5-(5-(6-aminopyridin-2- yl)-2-chlorobenzamido)- N-(1,3-dihydroxypropan- 2-yl)-1-phenyl-1H- pyrazole-3-carboxamide | 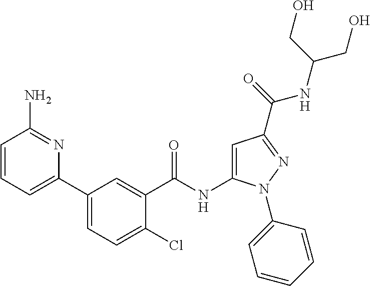 [SMILES: Nc1cccc(-c2ccc(Cl)c(C(=O)Nc3cc(C(=O)NC(CO)CO)nn3-c3ccccc3)c2)n1] | ¹H NMR (400 MHz, MeOH- d₄): δ ppm 3.74 (t, 4H), 4.16 (t, 1H), 6.56 (d, 1H), 7.05 (d, 2H), 7.47-7.53 (m, 5H), 7.63 (m, 2H), 7.95-8.00 (m, 2H). LCMS Rt = 2.31 minutes MS m/z 507 [M + H]⁺ Aminopropane-1,3-diol. PM A. |
| 110 | 5-(5-(6-aminopyridin-2- yl)-2-chlorobenzamido)- N-((3R,4S)-4- hydroxytetrahydrofuran- 3-yl)-1-phenyl-1H- pyrazole-3-carboxamide | 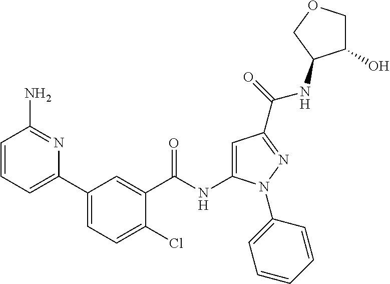 [SMILES: Nc1cccc(-c2ccc(Cl)c(C(=O)Nc3cc(C(=O)N[C@H]4COC[C@@H]4O)nn3-c3ccccc3)c2)n1] | ¹H NMR (400 MHz, MeOH- d₄): δ ppm 3.69 (d, 1H), 3.79 (dd, 1H), 4.05 (dd, 1H), 4.16 (dd, 1H), 4.36-4.39 (m, 2H), 6.56 (d, 1H), 7.02-7.06 (m, 2H), 7.47-7.56 (m, 5H), 7.62-7.64 (m, 2H), 7.94-8.00 (m, 2H). LCMS Rt = 2.02 minutes MS m/z 519 [M + H]⁺ (3R,4S)-4- Aminotetrahydrofuran-3-ol. PM B. |
| 111 | 5-(5-(6-aminopyridin-2- yl)-2-chlorobenzamido)- N-(2-hydroxy-2- methylpropyl)-1-phenyl- 1H-pyrazole-3- carboxamide | 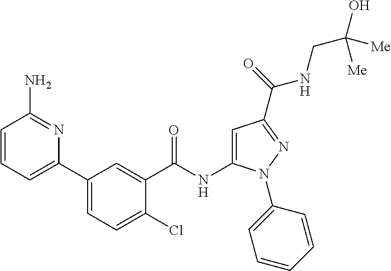 [SMILES: CC(C)(O)CNC(=O)c1cc(NC(=O)c2cc(-c3cccc(N)n3)ccc2Cl)n(-c2ccccc2)n1] | ¹H NMR (400 MHz, CDCl₃): δ ppm 1.26 (s, 6H), 2.56 (br. s, 1H), 3.43 (d, 2H), 4.51 (br s, 1H), 6.47 (d, 1H), 7.08 (d, 1H), 7.29 (t, 1H), 7.35-7.41 (m, 2H), 7.47-7.55 (m, 5H), 7.98 (d, 1H), 8.41 (br s, 2H). LCMS Rt = 2.13 minutes MS m/z 505 [M + H]⁺ 1-amino-2-methylpropan-2- ol. PM B. |
| 112 | Racemic-5-(5-(6- aminopyridin-2-yl)-2- chlorobenzamido)-N-(2- hydroxy-3- methoxypropyl)-1- phenyl-1H-pyrazole-3- carboxamide | 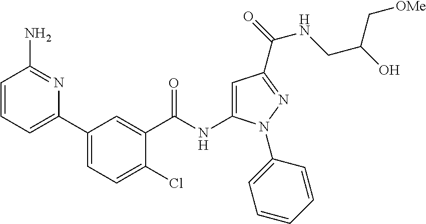 [SMILES: COCC(O)CNC(=O)c1cc(NC(=O)c2cc(-c3cccc(N)n3)ccc2Cl)n(-c2ccccc2)n1] | ¹H NMR (400 MHz, MeOH- d₄): δ ppm 3.39 (s, 3H), 3.42-3.48 (m, 3H), 3.56-3.61 (m, 1H), 3.91 (q, 1H), 6.57 (d, 1H), 7.04-7.05 (m, 2H), 7.48-7.58 (m, 5H), 7.63-7.65 (m, 2H), 7.95-8.01 (m, 2H). MS m/z 521 [M + H]⁺ Racemic-1-amino-3- methoxypropan-2-ol. PM B. |
| 113 | 5-(5-(6-aminopyridin-2- yl)-2-chlorobenzamido)- N-((1r,4r)-4- hydroxycyclohexyl)-1- phenyl-1H-pyrazole-3- carboxamide | 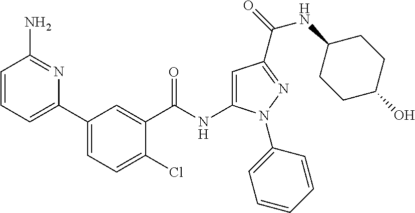 [SMILES: Nc1cccc(-c2ccc(Cl)c(C(=O)Nc3cc(C(=O)N[C@H]4CC[C@H](O)CC4)nn3-c3ccccc3)c2)n1] | ¹H NMR (400 MHz, MeOH- d₄): δ ppm 1.43 (m, 4H), 2.02 (m, 4H), 3.56 (s, 1H), 3.88 (m, 1H), 6.57 (d, 1H), 7.03 (d, 2H), 7.50-7.57 (m, 5H), 7.63 (d, 2H), 7.99 (m, 2H). LCMS Rt = 2.51 minutes MS m/z 531 [M + H]⁺ (1r,4r)-4-aminocyclohexan- 1-ol. |
| 114 | Racemic-5-(5-(6- aminopyridin-2-yl)-2- chlorobenzamido)-1- phenyl-N- (tetrahydrofuran-3-yl)- 1H-pyrazole-3- carboxamide | 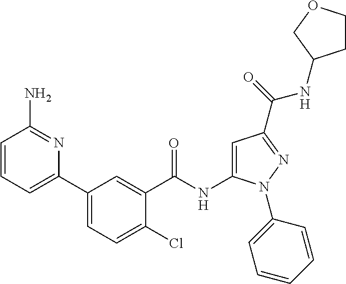 [SMILES: Nc1cccc(-c2ccc(Cl)c(C(=O)Nc3cc(C(=O)NC4CCOC4)nn3-c3ccccc3)c2)n1] | ¹H NMR (400 MHz, CDCl₃): δ ppm 2.00-1.92 (m, 1H), 2.18-2.09 (m, 1H), 3.60 (m, 1H), 3.74-3.68 (m, 1H), 3.87-3.81 (m, 2H), 4.55-4.43 (m, 1H), 6.09 (s, 2H), 6.48 (d, 1H), 6.95 (s, 1H), 7.11 (dd, 1H), 7.65-7.46 (m, 7H), 8.06 (d, 1H), 8.08 (s, 1H), 8.39 (d, 1H), 10.79 (s, 1H). LCMS Rt = 2.81 minutes MS m/z 503 [M + H]⁺ Racemic-tetrahydrofuran-3- amine. PM C. |

### Example 115

### Racemic-5-(5-(6-aminopyridin-2-yl)-2-chlorobenzamido)-N-(1-hydroxypropan-2-yl)-1-phenyl-1H-pyrazole-3-carboxamide

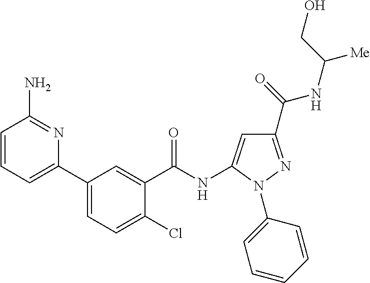 [SMILES: CC(CO)NC(=O)c1cc(NC(=O)c2cc(-c3cccc(N)n3)ccc2Cl)n(-c2ccccc2)n1]

The title compound was prepared according to the method described for Example 1 using 5-(5-(6-((tert-butoxycarbonyl)amino)pyridin-2-yl)-2-chlorobenzamido)-1-phenyl-1H-pyrazole-3-carboxylic acid (Preparation 1) and racemic-2-aminopropan-1-ol. The residue was dissolved in a solution of HCl in dioxane (0.2 mL, 0.84 mmol) and stirred at room temperature for 14 hours. The reaction was filtered, the solid was collected and dissolved in methanol. The solution was eluted through an SCX-2 column using MeOH followed by 7N NH₃ in MeOH.

to afford the title compound.

¹H NMR (400 MHz, MeOH-d₄): δ ppm 1.26 (d, 3H), 3.60 (d, 2H), 4.18 (q, 1H), 6.56 (d, 1H), 7.04 (d, 2H), 7.50-7.58 (m, 5H), 7.64 (d, 2H), 7.99 (t, 2H).

### Example 116

### 5-(5-(6-amino-3,5-difluoropyridin-2-yl)-2-chlorobenzamido)-N-(2-hydroxyethyl)-1-phenyl-1H-pyrazole-3-carboxamide

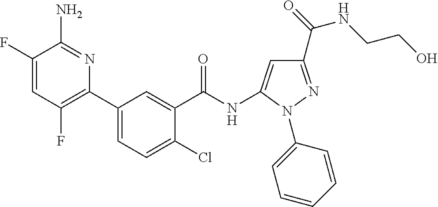 [SMILES: Nc1nc(-c2ccc(Cl)c(C(=O)Nc3cc(C(=O)NCCO)nn3-c3ccccc3)c2)c(F)cc1F]

Ethyl 5-(5-(6-amino-3,5-difluoropyridin-2-yl)-2-chlorobenzamido)-1-phenyl-1H-pyrazole-3-carboxylate (Example 144, 150 mg, 0.301 mmol) was stirred in ethanolamine (4 mL) at 60° C. for 18 hours. The reaction was concentrated in vacuo and partitioned between dichloromethane (25 mL) and water (15 mL). The aqueous layer was re-extracted with dichloromethane (15 mL). The combined organic layers were dried over anhydrous sodium sulphate and concentrated in vacuo. The residue was dissolved in MeOH (3 mL) and left standing to crystallise the title compound (72 mg, 47%).

¹H NMR (400 MHz, DMSO-d₆): δ ppm 3.30-3.34 (m, 2H), 3.47-3.51 (m, 2H), 4.71-4.74 (t, 1H), 6.38 (br s, 2H), 6.89 (s, 1H), 7.44-7.63 (m, 6H), 7.71-7.76 (t, 1H), 7.89-7.91 (m, 2H), 8.12-8.15 (m, 1H), 10.79 (br s, 1H).

LCMS Rt=2.80 minutes MS m/z 513 [M+H]⁺

### Example 117

### 4-(5-(6-amino-3,5-difluoropyridin-2-yl)-2-chlorobenzamido)-N-methyl-3-phenyl-1H-pyrazole-1-carboxamide

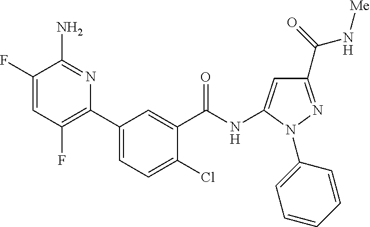 [SMILES: CNC(=O)c1cc(NC(=O)c2cc(-c3nc(N)c(F)cc3F)ccc2Cl)n(-c2ccccc2)n1]

A solution of ethyl 4-(5-(6-amino-3,5-difluoropyridin-2-yl)-2-chlorobenzamido)-3-phenyl-1H-pyrazole-1-carboxylate (Example 144, 0.6 g, 0.13 mmol) in methylamine (40% in MeOH) was stirred at 60° C. for 18 hours. The reaction was concentrated in vacuo and triturated with MeOH to afford the title compound (32 mg, 53%).

¹H NMR (400 MHz, DMSO-d₆): δ ppm 2.77 (d, 3H), 6.40 (br s, 2H), 6.89 (s, 1H), 7.46-7.49 (t, 1H), 7.54-7.58 (t, 2H), 7.60-7.65 (t, 3H), 7.73-7.78 (t, 1H), 7.90-7.94 (m, 2H), 8.25-8.28 (m, 1H), 10.79 (br s, 1H).

MS m/z 483 [M+H]⁺.

### Example 118

### 5-(5-(6-amino-3,5-difluoropyridin-2-yl)-2-chlorobenzamido)-N-(2-methoxyethyl)-1-phenyl-1H-pyrazole-3-carboxamide

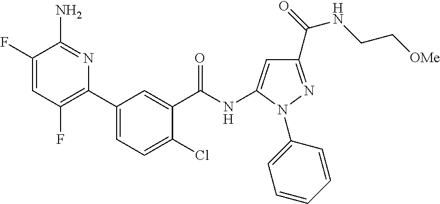 [SMILES: COCCNC(=O)c1cc(NC(=O)c2cc(-c3nc(N)c(F)cc3F)ccc2Cl)n(-c2ccccc2)n1]

The title compound was prepared according to the method described for Example 117 using 2-methoxyethanamine at 70° C. The residue was purified using preparative HPLC. ¹H NMR (400 MHz, DMSO-d₆): δ ppm 3.24 (s, 3H), 3.41 (m, 4H), 6.39 (s, 2H), 6.89 (s, 1H), 7.46 (m, 1H), 7.52-7.62 (m, 5H), 7.73 (t, 1H), 7.89 (m, 2H), 8.20 (t, 1H), 10.80 (s, 1H).

LCMS Rt=2.72 minutes MS m/z 527 [M+H]⁺

### Example 119

### (R)-5-{[5-(6-amino-3-fluoropyridin-2-yl)-2-chlorobenzoyl]amino}-N-(2-hydroxypropyl)-1-phenyl-1H-pyrazole-3-carboxamide

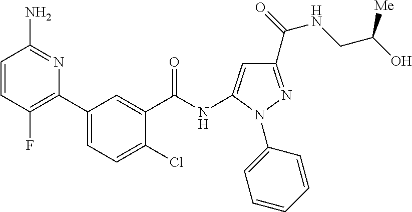 [SMILES: C[C@@H](O)CNC(=O)c1cc(NC(=O)c2cc(-c3nc(N)ccc3F)ccc2Cl)n(-c2ccccc2)n1]

To a suspension of ethyl 5-(5-(6-amino-3-fluoropyridin-2-yl)-2-chlorobenzamido)-1-phenyl-1H-pyrazole-3-carboxylate (Example 147, 47 mg, 0.1 mmol) and (R)-1-aminopropan-2-ol (22 mg, 0.29 mmol) in methanol (1 mL) was added DIPEA (0.05 mL, 0.29 mmol) and the reaction was heated to 50° C. for 18 hours. Lithium hydroxide (1.1 eq) was added and the reaction heated to 70° C. for 3 days. The reaction was partitioned between saturated aqueous sodium bicarbonate solution (100 mL) and ethyl acetate (2×50 mL). The organic layers were combined, washed with brine (100 mL), dried over magnesium sulphate and concentrated in vacuo. The resulting solid was triturated with TBME and pentane to afford the title compound.

¹H NMR (400 MHz, MeOH-d₄): δ ppm 1.21 (d, 3H), 3.31 (m, 1H), 3.47 (dd, 1H), 3.96 (m, 1H), 6.58 (dd, 1H), 7.03 (s, 1H), 7.38 (dd, 1H), 7.47-7.61 (m, 4H), 7.62-7.65 (m, 2H), 7.98-8.00 (m, 2H).

LCMS Rt=2.64 minutes MS m/z 509 [M+H]⁺

### Example 120

### 5-(5-(6-amino-3-fluoropyridin-2-yl)-2-chlorobenzamido)-N-cyclopropyl-1-phenyl-1H-pyrazole-3-carboxamide

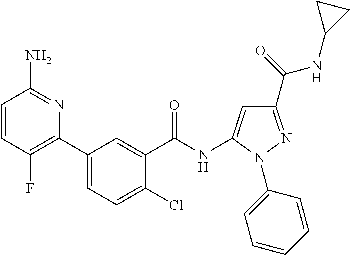 [SMILES: Nc1ccc(F)c(-c2ccc(Cl)c(C(=O)Nc3cc(C(=O)NC4CC4)nn3-c3ccccc3)c2)n1]

To a solution of 5-(5-bromo-2-chlorobenzamido)-N-cyclopropyl-1-phenyl-1H-pyrazole-3-carboxamide (Preparation 12, 150 mg, 0.32 mmol) degassed dioxane (2 mL) was added bis(pinacolato)diboron (91 mg, 0.35 mmol) and potassium acetate (94 mg, 0.96 mmol). The reaction was further degassed for 15 minutes before the addition of Pd(dppf)Cl₂ (13 mg, 5% mol) and heating to 100° C. for 18 hours. The reaction was filtered through arbocel and concentrated in vacuo. The residue was dissolved in dioxane (2 mL) and water (2 mL). To the solution was added 6-chloro-5-fluoropyridin-2-amine (93 mg, 0.64 mmol) and potassium carbonate (88 mg, 0.64 mmol). The reaction was degassed for 15 minutes followed by the addition of Pd(PPh₃)₄ (37 mg, 10% mol). The reaction was degassed for a further 5 minutes then heated at 100° C. for 3 hours. The reaction was cooled and partitioned between EtOAc (10 mL) and water (5 mL). The organic layer was collected, dried over MgSO₄ and concentrated in vacuo. The residue was purified using reverse phase preparative HPLC followed by elution through an SCX-2 column using MeOH followed by 7N NH₃ in MeOH.

to afford the title compound (30 mg, 20%).

¹H NMR (400 MHz, MeOH-d₄): δ ppm 0.66 (m, 2H), 0.81 (m, 2H), 2.86 (m, 1H), 6.58 (dd, 1H), 7.02 (s, 1H), 7.38 (t, 1H), 7.46-7.65 (m, 6H), 7.99 (s, 2H).

MS m/z 491 [M+H]⁺

### Example 121

### 5-(5-(6-amino-3-fluoropyridin-2-yl)-2-chlorobenzamido)-N-(2-hydroxyethyl)-1-phenyl-1H-pyrazole-3-carboxamide

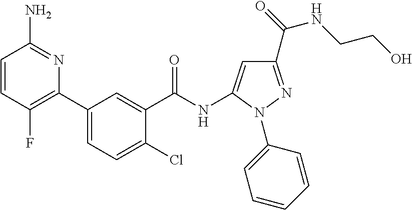 [SMILES: Nc1ccc(F)c(-c2ccc(Cl)c(C(=O)Nc3cc(C(=O)NCCO)nn3-c3ccccc3)c2)n1]

A solution of N-(2-((tert-butyldimethylsilyl)oxy)ethyl)-5-(2-chloro-5-(4,4,5,5-tetramethyl-1,3,2-dioxaborolan-2-yl)benzamido)-1-phenyl-1H-pyrazole-3-carboxamide (Preparation 7, 500 mg, 0.80 mmol), 6-chloro-5-fluoropyridin-2-amine (234 mg, 1.60 mmol) and potassium carbonate (221 mg, 1.60 mmol) in dioxane (10 mL) and water (5 mL) was degassed with nitrogen for 10 minutes before the addition of Pd(PPh₃)₄ (92 mg, 0.080 mmol). The reaction was heated to reflux for 5 hours. The reaction was diluted with water (20 mL) and EtOAc (20 mL). The aqueous phase was further extracted with EtOAc (3×20 mL) and the combined organic layers were dried over MgSO₄ and concentrated in vacuo. The residue was purified by silica gel column chromatography eluting with 1:1 EtOAc:heptanes and dissolved in THF (5 mL). TBAF (1M in THF, 0.36 mL, 0.36 mmol) was added and the reaction stirred at room temperature for 18 hours. The reaction was quenched by the addition of saturated aqueous ammonium chloride solution (20 mL) and extracted into EtOAc (3×10 mL). The combined organic extracts were dried over magnesium sulphate and concentrated in vacuo. The residue was purified using reverse phase chromatography eluting with 5-80% MeCN in water (with 0.1% ammonia) followed by trituration with MeCN to afford the title compound (12 mg, 10%).

¹H NMR (400 MHz, DMSO-d₆): δ ppm 3.31-3.36 (m, 3H), 3.50 (m, 2H), 4.76 (t, 1H), 6.09 (s, 2H), 6.52 (dd, 1H), 6.91 (s, 1H), 7.45-7.50 (m, 2H), 7.54-7.64 (m, 4H), 7.96-7.97 (m, 2H), 8.17 (t, 2H), 10.81 (s, 1H).

MS m/z 495 [M+H]⁺

### Example 122

### 5-(((5-(6-amino-3-chloropyridin-2-yl)-2-chlorophenyl)(hydroxy)methyl)amino)-N-(2-hydroxyethyl)-1-phenyl-1H-pyrazole-3-carboxamide

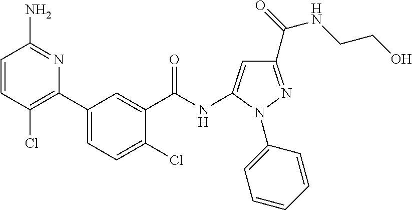 [SMILES: Nc1ccc(Cl)c(-c2ccc(Cl)c(C(=O)Nc3cc(C(=O)NCCO)nn3-c3ccccc3)c2)n1]

A solution of N-(2-((tert-butyldimethylsilyl)oxy)ethyl)-5-(2-chloro-5-(4,4,5,5-tetramethyl-1,3,2-dioxaborolan-2-yl)benzamido)-1-phenyl-1H-pyrazole-3-carboxamide (Preparation 7, 500 mg, 0.80 mmol), 6-bromo-5-chloropyridin-2-amine (332 mg, 1.60 mmol) and potassium carbonate (221 mg, 1.60 mmol) in dioxane (10 mL) and water (5 mL) was degassed with nitrogen for 10 minutes before the addition of Pd(PPh₃)₄ (92 mg, 0.80 mmol). The reaction was heated to reflux for 2 hours. The reaction was diluted with EtOAc (20 mL) and water (20 mL). The aqueous phase was extracted with EtOAc (3×20 mL) and the combined organic layers were dried over MgSO₄ and concentrated in vacuo. The residue was purified by silica gel column chromatography eluting with 40-60% EtOAc in heptanes and dissolved in THF (2 mL). TBAF (1M in THF, 96 uL, 0.096 mmol) was added and the reaction stirred at room temperature for 18 hours. The reaction was quenched by the addition of saturated aqueous NH₄Cl solution (10 mL) and extracted into EtOAc (3×10 mL). The combined organic layers were dried over MgSO₄ and concentrated in vacuo. The residue was purified using reverse phase chromatography eluting with 5-50% MeCN in water with 0.1% ammonia followed by trituration with MeCN to afford the title compound (8 mg, 8%).

¹H NMR (400 MHz, DMSO-d₆): δ ppm 3.31-3.35 (2H, m), 3.50 (2H, q), 4.75 (t, 2H), 6.33 (s, 2H), 6.50 (d, 1H), 6.90 (s, 1H), 7.45-7.49 (m, 1H), 7.53-7.62 (m, 5H), 7.70-7.76 (m, 2H), 8.16 (t, 1H), 10.79 (br s, 1H).

MS m/z 511 [M+H]⁺

### Example 123

### 5-(5-(6-amino-5-fluoropyridin-2-yl)-2-chlorobenzamido)-N-(2-hydroxyethyl)-1-phenyl-1H-pyrazole-3-carboxamide

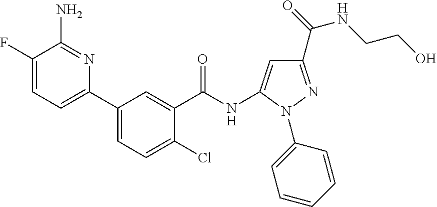 [SMILES: Nc1nc(-c2ccc(Cl)c(C(=O)Nc3cc(C(=O)NCCO)nn3-c3ccccc3)c2)ccc1F]

The title compound was prepared according to the method described for Example 122 using 6-bromo-3-fluoropyridin-2-amine. The residue was purified using silica gel column chromatography eluting with EtOAc followed by trituration with TBME.

¹H NMR (400 MHz, MeOH-d₄): δ ppm 3.53 (t, 2H), 3.72 (t, 2H), 7.03 (s, 1H), 7.07 (dd, 1H), 7.34-7.37 (m, 1H), 7.49-7.58 (m, 4H), 7.63-7.65 (m, 2H), 7.99 (dd, 1H), 8.03 (d, 1H).

MS m/z 495 [M+H]⁺

### Example 124

### 5-(5-(6-aminopyridin-2-yl)-2,4-dichlorobenzamido)-1-phenyl-1H-pyrazole-3-carboxamide

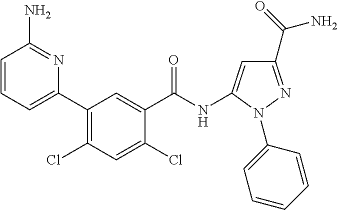 [SMILES: NC(=O)c1cc(NC(=O)c2cc(-c3cccc(N)n3)c(Cl)cc2Cl)n(-c2ccccc2)n1]

Ethyl 5-(5-(6-aminopyridin-2-yl)-2,4-dichlorobenzamido)-1-phenyl-1H-pyrazole-3-carboxylate (Example 145, 43 mg, 0.086 mmol) was dissolved in MeOH (1 mL) and NH₄OH solution (3 mL) and the reaction was heated at 50° C. for 9 hours. The reaction was cooled to room temperature and concentrated in vacuo. The residue was purified by reverse phase column chromatography eluting with 5-50% MeCN in water (0.1% NH₃) to afford the title compound (15 mg, 37%).

¹H NMR (400 MHz, MeOH-d₄): δ ppm 6.62 (d, 1H), 6.80 (d, 1H), 7.02 (s, 1H), 7.47-7.66 (m, 8H).

LCMS Rt=2.35 minutes MS m/z 467 [M+H]⁺

### Example 125

### 5-(5-(6-amino-3-cyano-5-fluoropyridin-2-yl)-2-chlorobenzamido)-1-phenyl-1H-pyrazole-3-carboxamide

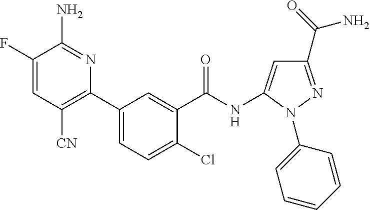 [SMILES: N#Cc1cc(F)c(N)nc1-c1ccc(Cl)c(C(=O)Nc2cc(C(N)=O)nn2-c2ccccc2)c1]

To a solution of ethyl 5-(5-(6-amino-3-cyano-5-fluoropyridin-2-yl)-2-chlorobenzamido)-1-phenyl-1H-pyrazole-3-carboxylate (Example 146, 100 mg, 0.198 mmol) in methanol (5 mL) was added ammonia in water (2 mL). The reaction was heated to 50° C. and stirred for 18 hours. The reaction was concentrated in vacuo and the resulting solid was triturated with MeOH to afford the title compound (42 mg, 45%).

¹H NMR (400 MHz, DMSO-d₆): δ ppm 6.87 (s, 1H), 7.37 (s, 1H), 7.45 (t, 1H), 7.50-7.70 (m, 8H), 7.78 (d, 1H), 7.86 (dd, 1H), 7.97 (d, 1H), 10.84 (br s, 1H).

LCMS Rt=2.64 minutes MS m/z 476 [M+H]⁺

### Example 126

### 5-(5-(6-aminopyridin-2-yl)-2-chlorobenzamido)-1-phenyl-1H-pyrazole-3-carboxamide

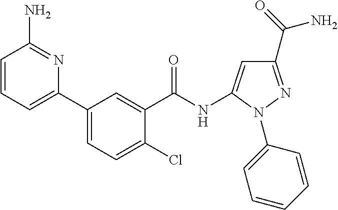 [SMILES: NC(=O)c1cc(NC(=O)c2cc(-c3cccc(N)n3)ccc2Cl)n(-c2ccccc2)n1]

To a solution of 5-(6-aminopyridin-2-yl)-2-chloro-N-(3-cyano-1-phenyl-1H-pyrazol-5-yl)benzamide (Example 151, 90 mg, 0.22 mmol) in dimethylsulfoxide (2.5 mL) was added potassium carbonate (60 mg, 0.44 mmol) followed by a 30% aqueous hydrogen peroxide solution (500 uL, 4.34 mmol), and the reaction was stirred at room temperature for 1 hour. The reaction was diluted with ethyl acetate (65 mL) and washed with brine.

The organic layer was dried over magnesium sulphate and concentrated in vacuo. The residue was purified using reverse phase column chromatography eluting with 0-60% acetonitrile in water to afford the title compound (28 mg, 30%).

¹H NMR (400 MHz, Acetone-d₆): δ ppm 5.58 (br s, 1H), 6.58 (m, 2H), 7.13 (m, 1H), 7.23 (br s, 1H), 7.40-7.60 (m, 5H), 7.67 (m, 2H), 8.07 (d, 1H), 8.22 (s, 1H), 9.75 (br s, 1H).

MS m/z 433 [M+H]⁺

### Example 127

### 5-(5-(6-amino-3,5-difluoropyridin-2-yl)-2-chlorobenzamido)-1-phenyl-1H-pyrazole-3-carboxamide

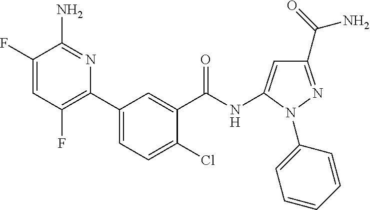 [SMILES: NC(=O)c1cc(NC(=O)c2cc(-c3nc(N)c(F)cc3F)ccc2Cl)n(-c2ccccc2)n1]

To a solution of 5-(6-amino-3,5-difluoropyridin-2-yl)-2-chloro-N-(3-cyano-1-phenyl-1H-pyrazol-5-yl)benzamide (Example 152, 100 mg, 0.221 mmol) in DMSO (2 mL) was added K₂CO₃ (61 mg, 0.443 mmol) followed by the addition of H₂O₂ (35% in water, 0.489 mL, 5.55 mmol). The reaction was stirred at room temperature for 2 hours. Water (10 mL) was added and the resulting solid extracted with EtOAc (25 mL), washed with water (3×2 mL), brine (10 mL), dried over sodium sulphate and concentrated in vacuo. The residue was triturated in MeOH to afford the title compound.

¹H NMR (400 MHz, DMSO-d₆): δ ppm 6.40 (s, 2H), 6.90 (s, 1H), 7.37-7.78 (m, 9H), 7.91 (s, 1H), 7.93 (s, 1H), 10.79 (s, 1H).

LCMS Rt=2.67 minutes MS m/z 469 [M+H]⁺

### Example 128

### 5-(5-(6-amino-5-chloropyridin-2-yl)-2-chlorobenzamido)-1-phenyl-1H-pyrazole-3-carboxamide

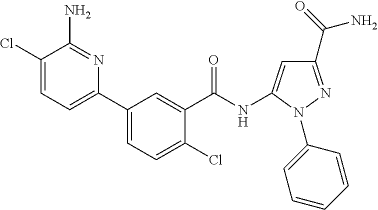 [SMILES: NC(=O)c1cc(NC(=O)c2cc(-c3ccc(Cl)c(N)n3)ccc2Cl)n(-c2ccccc2)n1]

The title compound was prepared according to the method described for Example 127 using tert-butyl (6-(3-((3-carbamoyl-1-phenyl-1H-pyrazol-5-yl)carbamoyl)-4-chlorophenyl)-3-chloropyridin-2-yl)carbamate (Preparation 4). The residue was dissolved in DCM (10 mL), treated with trifluoroacetic acid (0.572 mL) and stirred at room temperature for 3 hours. The reaction was concentrated in vacuo, azeotroped with DCM and eluted through an SCX-2 column using 7N NH₃ in MeOH to afford the title compound.

¹H NMR (400 MHz, DMSO-d₆): δ ppm 6.43 (s, 2H), 6.88 (s, 1H), 7.17 (d, 1H), 7.34 (s, 1H), 7.46-7.43 (m, 1H), 7.69-7.55 (m, 8H), 8.07 (br s, 1H).

LCMS Rt=2.87 minutes MS m/z 467 [M+H]⁺

### Example 129

### 5-(5-(6-amino-3-chloro-5-fluoropyridin-2-yl)-2-chlorobenzamido)-1-phenyl-1H-pyrazole-3-carboxamide

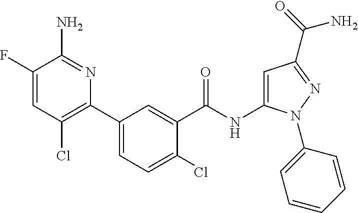 [SMILES: NC(=O)c1cc(NC(=O)c2cc(-c3nc(N)c(F)cc3Cl)ccc2Cl)n(-c2ccccc2)n1]

The title compound was prepared according to the method described for Example 127 using 5-(6-amino-3-chloro-5-fluoropyridin-2-yl)-2-chloro-N-(3-cyano-1-phenyl-1H-pyrazol-5-yl)benzamide (Example 150).

¹H NMR (400 MHz, MeOH-d₄): δ ppm 7.01 (s, 1H), 7.45-7.56 (m, 5H), 7.62 (m, 2H), 7.74 (m, 2H).

LCMS Rt=2.89 minutes MS m/z 485 [M+H]⁺

### Example 130

### 5-(5-(6-amino-5-fluoropyridin-2-yl)-2-chlorobenzamido)-1-phenyl-1H-pyrazole-3-carboxamide

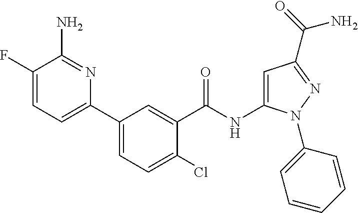 [SMILES: NC(=O)c1cc(NC(=O)c2cc(-c3ccc(F)c(N)n3)ccc2Cl)n(-c2ccccc2)n1]

To a solution of 5-(2-chloro-5-(4,4,5,5-tetramethyl-1,3,2-dioxaborolan-2-yl)benzamido)-1-phenyl-1H-pyrazole-3-carboxamide (Preparation 10, 150 mg, 0.32 mmol), 6-chloro-3-fluoropyridin-2-amine (47 mg, 0.32 mmol) and CsF (146 mg, 0.96 mmol) in methanol (5 mL) was added Pd(dppf)Cl₂ (13 mg, 0.02 mmol) and the reaction was heated to 120° C. for 1 hour under microwave irradiation. The reaction was cooled to room temperature and concentrated in vacuo. The residue was eluted through a silica plug using 10% methanol in DCM followed by elution through an SCX-2 column using 10% methanol in DCM followed by NH₃ in methanol. The residue was further purified using silica gel column chromatography eluting with 5% methanol in DCM followed by trituration with TBME to afford the title compound (9 mg, 6%).

¹H NMR (400 MHz, DMSO-d₆): δ ppm 6.40 (br s, 2H), 6.90 (s, 1H), 7.14 (dd, 1H), 7.39 (br s, 1H), 7.48 (m, 2H), 7.57 (m, 3H), 7.63 (m, 2H), 7.70 (br s, 1H), 8.04 (m, 2H), 10.78 (br s, 1H).

LCMS Rt=2.46 minutes MS m/z 451 [M+H]⁺

### Example 131

### 5-(5-(6-amino-3-chloropyridin-2-yl)-2-chlorobenzamido)-1-phenyl-1H-pyrazole-3-carboxamide

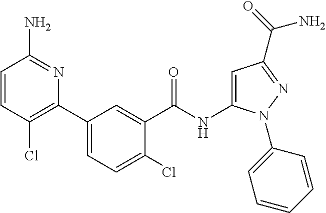 [SMILES: NC(=O)c1cc(NC(=O)c2cc(-c3nc(N)ccc3Cl)ccc2Cl)n(-c2ccccc2)n1]

To a solution of 5-(2-chloro-5-(4,4,5,5-tetramethyl-1,3,2-dioxaborolan-2-yl)benzamido)-1-phenyl-1H-pyrazole-3-carboxamide (Preparation 10, 222 mg, 0.48 mmol) in dioxane (5 mL was added potassium carbonate (131 mg, 0.95 mmol), 6-bromo-5-chloropyridin-2-amine (129 mg, 0.62 mmol) and water (3 mL). The reaction was degassed followed by the addition of Pd(dppf)Cl₂ (19 mg, 0.02 mmol) and heated to reflux for 18 hours. The reaction mixture was cooled and concentrated in vacuo. The residue was partitioned between ethyl acetate (20 mL) and brine (20 mL). The aqueous layer was extracted with ethyl acetate and the organic layers combined dried over sodium sulphate and concentrated in vacuo. The residue was purified using silica gel column chromatography eluting with 50-100% EtOAc in heptanes 1:1 followed by 5% MeOH in EtOAc. Final purification using preparative HPLC gave the title compound as a white solid (6 mg, 3%).

¹H NMR (400 MHz, MeOH-d₄): δ ppm 6.59 (d, 1H), 7.02 (s, 1H), 7.50-7.60 (m, 5H), 7.61-7.69 (m, 2H), 7.77-7.82 (m, 2H).

LCMS Rt=2.70 minutes MS m/z 467 [M+H]⁺

### Example 132

### 5-(5-(6-amino-3-methoxypyridin-2-yl)-2-chlorobenzamido)-1-phenyl-1H-pyrazole-3-carboxamide

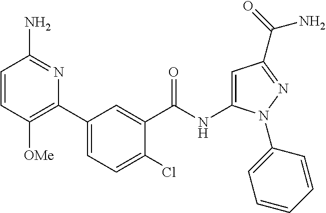 [SMILES: COc1ccc(N)nc1-c1ccc(Cl)c(C(=O)Nc2cc(C(N)=O)nn2-c2ccccc2)c1]

The title compound was prepared according to the method described for Example 131 using 6-bromo-5-methoxypyridin-2-amine. The residue was purified using silica gel column chromatography eluting with 3-4% MeOH in DCM followed by preparative HPLC.

¹H NMR (400 MHz, DMSO-d₆): δ ppm 3.71 (s, 3H), 5.66 (s, 2H), 6.53 (d, 1H), 6.89 (s, 1H), 7.55-7.38 (m, 9H), 8.02 (d, 2H) 10.73 (br s, 1H).

LCMS Rt=2.72 minutes MS m/z 463 [M+H]⁺

### Example 133

### 5-(5-(6-aminopyridin-2-yl)-2-chlorobenzamido)-1-(3-hydroxyphenyl)-1H-pyrazole-3-carboxamide

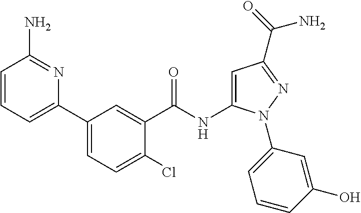 [SMILES: NC(=O)c1cc(NC(=O)c2cc(-c3cccc(N)n3)ccc2Cl)n(-c2cccc(O)c2)n1]

The title compound was prepared according to the method described for Example 122 using 1-(3-(benzyloxy)phenyl)-5-(2-chloro-5-(4,4,5,5-tetramethyl-1,3,2-dioxaborolan-2-yl)benzamido)-1H-pyrazole-3-carboxamide (Preparation 9) and 6-bromopyridin-2-amine. The residue (59 mg, 0.11 mmol) was dissolved in DCM (2 mL) and DMF (3 drops) and treated with boron tribromide (1M in dichloromethane; 1 mL, 1.09 mmol) dropwise. The reaction was stirred at room temperature for 20 hours then quenched with methanol and concentrated in vacuo. The residue was purified by preparative HPLC to afford the title compound as a colourless solid (6 mg, 10% yield).

¹H NMR (400 MHz, MeOH-d₄): δ ppm 6.56 (d, 1H), 6.91 (dd, 1H), 7.02-7.10 (m, 4H), 7.35 (t, 1H), 7.51-7.55 (m, 2H), 7.97 (dd, 1H), 8.01 (d, 1H).

MS m/z 449 [M+H]⁺

### Example 134

### 5-(5-(6-amino-5-methylpyridin-2-yl)-2-chlorobenzamido)-1-phenyl-1H-pyrazole-3-carboxamide

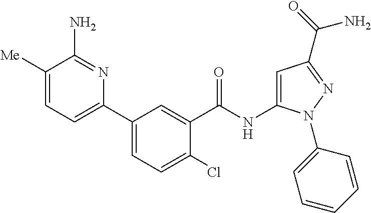 [SMILES: Cc1ccc(-c2ccc(Cl)c(C(=O)Nc3cc(C(N)=O)nn3-c3ccccc3)c2)nc1N]

To a solution of ethyl 5-(5-(6-(tert-butylamino)-5-methylpyridin-2-yl)-2-chlorobenzamido)-1-phenyl-1H-pyrazole-3-carboxylate (Preparation 2, 0.163 g, 0.31 mmol) in methanol was added 35% aqueous ammonia (40 mL) and the reaction was heated to 60° C. for 3 days. The reaction was concentrated in vacuo and dissolved in DCM (10 mL). The solution was treated with TFA (5 mL) and stirred at room temperature for 18 hours. The reaction was concentrated in vacuo and purified using preparative HPLC.

¹H NMR (400 MHz, MeOH-d₄): δ ppm 2.20 (s, 3H), 7.00 (m, 1H), 7.40-7.70 (m, 8H), 8.00 (m, 2H).

LCMS Rt=3.56 minutes MS m/z 447 [M+H]⁺

### Example 135

### 5-(5-(6-amino-3-fluoropyridin-2-yl)-2-chlorobenzamido)-1-phenyl-1H-pyrazole-3-carboxamide

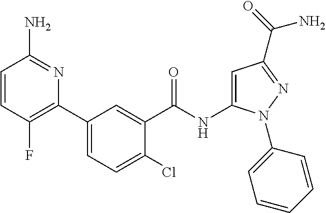 [SMILES: NC(=O)c1cc(NC(=O)c2cc(-c3nc(N)ccc3F)ccc2Cl)n(-c2ccccc2)n1]

A solution of 5-(2-chloro-5-(4,4,5,5-tetramethyl-1,3,2-dioxaborolan-2-yl)benzamido)-1-phenyl-1H-pyrazole-3-carboxamide (Preparation 10, 50 mg, 0.11 mmol), 6-chloro-5-fluoropyridin-2-amine (47 mg, 0.32 mmol), tetrakis(triphenylphosphine)palladium (12 mg, 0.01 mmol) and potassium carbonate (30 mg, 0.21 mmol) in dioxane (1.5 mL) and water (1.5 mL) was heated at 110° C. under microwave irradiation for 1 hour. The reaction was poured into water (25 mL) and extracted with ethyl acetate (2×25 mL). The organic layer was washed with brine (10 mL), dried over magnesium sulphate and concentrated in vacuo.

The residue was purified using silica gel column chromatography eluting with ethyl acetate followed by 10% MeOH in DCM. The resulting solid was triturated with EtOAc to afford the title compound (15 mg, 31%).

¹H NMR (400 MHz, DMSO-d₆): δ ppm 6.07 (s, 2H), 6.51 (m, 1H), 6.90 (s, 1H), 7.68-7.37 (m, 9H), 7.96 (s, 2H), 10.78 (s, 1H).

LCMS Rt=2.53 minutes MS m/z 451 [M+H]⁺

### Example 136

### 5-(5-(6-aminopyridin-2-yl)-2,4-dichlorobenzamido)-N-(2-hydroxyethyl)-1-phenyl-1H-pyrazole-3-carboxamide

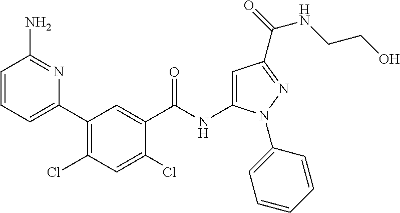 [SMILES: Nc1cccc(-c2cc(C(=O)Nc3cc(C(=O)NCCO)nn3-c3ccccc3)c(Cl)cc2Cl)n1]

A mixture of ethyl 5-(5-(6-((tert-butoxycarbonyl)amino)pyridin-2-yl)-2,4-dichlorobenzamido)-1-phenyl-1H-pyrazole-3-carboxylate (Preparation 5, 300 mg, 0.503 mmol) and 2-aminoethanol (921 mg, 15.1 mmol) was stirred at 50° C. for 2 hours. The reaction was cooled to room temperature and treated with water (15 mL). The solution was extracted with ethyl acetate (2×25 mL), the organic layers were combined, dried over sodium sulphate and concentrated in vacuo. The residue was purified using silica gel column chromatography eluting with 50-100% ethyl acetate in heptanes before dissolving in DCM (0.6 mL). The solution was treated with TFA (0.6 mL) and stirred at room temperature for 3 hours. The mixture was concentrated in vacuo and dissolved in methanol (2 mL). 2N aqueous NaOH was added and the solution was stirred for 1 hour before acidifying to pH=2 with 2N HCl. The solution was eluted through an SCX-2 column using 50% aqueous MeOH followed by 2N NH₃ in MeOH to afford the title compound as a white solid (76 mg, 83%).

¹H NMR (400 MHz, DMSO-d₆): δ ppm 3.31 (q, 2H), 3.48 (q, 2H), 4.73 (t, 1H), 6.08 (s, 2H), 6.48 (d, 1H), 6.74 (d, 1H), 6.88 (s, 1H), 7.41-7.70 (m, 7H), 7.77 (s, 1H), 8.11 (t, 1H), 10.77 (s, 1H).

LCMS Rt=2.54 minutes MS m/z 511 [M+H]⁺

### Example 137

### (R)-5-(5-(6-aminopyridin-2-yl)-2-chlorobenzamido)-N-(5-oxopyrrolidin-3-yl)-1-phenyl-1H-pyrazole-3-carboxamide

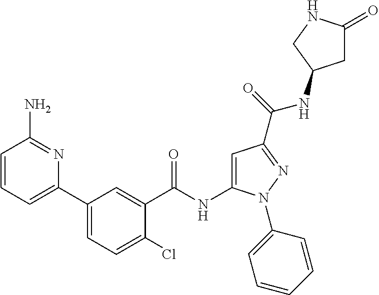 [SMILES: Nc1cccc(-c2ccc(Cl)c(C(=O)Nc3cc(C(=O)N[C@H]4CNC(=O)C4)nn3-c3ccccc3)c2)n1]

The title compound was prepared according to Library Protocol 1 using (R)-4-aminopyrrolidin-2-one.

Rt=1.32 minutes MS m/z 516 [M+H]⁺

### Example 138

### 5-(5-(6-aminopyridin-2-yl)-2-chlorobenzamido)-1-phenyl-1H-pyrazole-3-carboxylic Acid

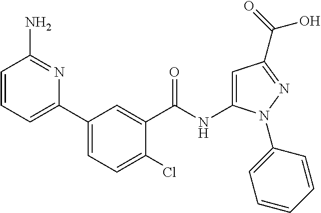 [SMILES: Nc1cccc(-c2ccc(Cl)c(C(=O)Nc3cc(C(=O)O)nn3-c3ccccc3)c2)n1]

To a solution of ethyl 5-(5-(6-aminopyridin-2-yl)-2-chlorobenzamido)-1-phenyl-1H-pyrazole-3-carboxylate (Example 143, 91 mg, 0.19 mmol) dissolved in methanol (5 mL) was added LiOH (20 mg, 0.83 mmol) and water (1 mL) and stirred at room temperature for 18 hours. Two drops of formic acid were added to neutralise the reaction and the reaction was concentrated in vacuo to afford the title compound as an oil (217 mg) that was used directly in the next step.

¹H NMR (400 MHz, DMSO-d₆): δ ppm 6.39 (d, 1H), 6.86 (br s, 1H), 7.00 (d, 1H), 7.07 (t, 1H), 7.29 (m, 3H), 7.43 (t, 1H), 7.73 (dd, 1H), 8.13 (m, 3H).

LCMS Rt=1.89 minutes MS m/z 432 [M−H]⁻

The following Examples were prepared according to the Method described by Example 138 using the appropriate ethyl ester as described below at room temperature for 18 hours or at 40° C. for 2 hours. The crude residues were purified as above or according to the purification method described below:

Purification Method A: The reaction was acidified with 2M HCl to precipitate a white solid that was filtered and washed with water and TBME or IMS.

| Ex | | | |
|---|---|---|---|
| Number | Name | Structure | Data/Ester/PM |
| 139 | 5-(5-(6-amino-3,5- difluoropyridin-2-yl)-2- chlorobenzamido)-1- phenyl-1H-pyrazole-3- carboxylic acid | 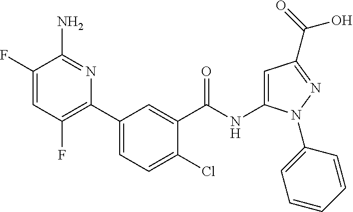 [SMILES: Nc1nc(-c2ccc(Cl)c(C(=O)Nc3cc(C(=O)O)nn3-c3ccccc3)c2)c(F)cc1F] | ¹H NMR (400 MHz, DMSO-d₆): δ ppm 6.38 (br s, 2H), 6.94 (s, 1H), 7.45-7.63 (m, 6H), 7.71 (t, 1H), 7.89-7.91 (m, 2H), 10.82 (s, 1H). MS m/z 470 [M + H]⁺ Ethyl 5-(5-(6-amino-3,5- difluoropyridin-2-yl)-2- chlorobenzamido)-1-phenyl-1H- pyrazole-3-carboxylate (Example 144). PM A. |
| 140 | 5-(5-(6-amino-3- fluoropyridin-2-yl)-2- chlorobenzamido)-1- phenyl-1H-pyrazole-3- carboxylic acid | 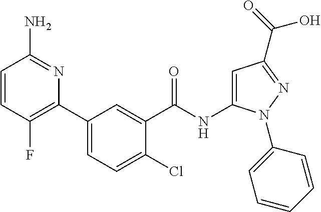 [SMILES: Nc1ccc(F)c(-c2ccc(Cl)c(C(=O)Nc3cc(C(=O)O)nn3-c3ccccc3)c2)n1] | ¹H NMR (400 MHz, DMSO-d₆): δ ppm 6.61 (d, 1H), 6.94 (s, 1H), 7.47-7.65 (m, 7H), 7.92- 7.94 (m, 2H), 10.84 (s, 1H). MS m/z 452 [M + H]⁺ ethyl 5-(5-(6-amino-3- fluoropyridin-2-yl)-2- chlorobenzamido)-1-phenyl-1H- pyrazole-3-carboxylate (Example 147). PM A. |
| 141 | 5-(5-(6-amino-3- chloropyridin-2-yl)-2- chlorobenzamido)-1- phenyl-1H-pyrazole-3- carboxylic acid | 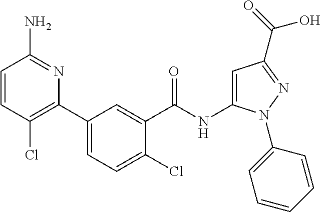 [SMILES: Nc1ccc(Cl)c(-c2ccc(Cl)c(C(=O)Nc3cc(C(=O)O)nn3-c3ccccc3)c2)n1] | ¹H NMR (400 MHz, DMSO-d₆): δ ppm 6.30 (br s, 1H), 6.49 (d, 1H), 6.93 (s, 1H), 7.45-7.48 (m, 1H), 7.51-7.59 (m, 6H), 7.69 (d, 1H), 7.73 (dd, 1H), 10.79 (br s, 1H), 12.96 (br s, 1H). MS m/z 468 [M + H]⁺ Ethyl 5-(5-(6-amino-3- chloropyridin-2-yl)-2- chlorobenzamido)-1-phenyl-1H- pyrazole-3-carboxylate (Example 148). PM A. |
| 142 | 5-(5-(6-amino-5- fluoropyridin-2-yl)-2- chlorobenzamido)-1- phenyl-1H-pyrazole-3- carboxylic acid | 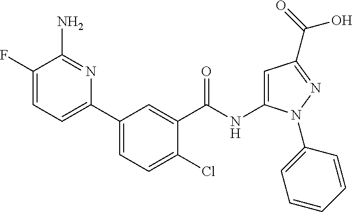 [SMILES: Nc1nc(-c2ccc(Cl)c(C(=O)Nc3cc(C(=O)O)nn3-c3ccccc3)c2)ccc1F] | ¹H NMR (400 MHz, DMSO-d₆): δ ppm 6.39 (s, 2H), 6.96 (s, 1H), 7.14 (dd, 1H), 7.45-7.48 (m, 2H), 7.58-7.61 (m, 5H), 8.06 (s, 2H), 10.81 (s, 1H), 13.00 (s, 1H). MS m/z 452 [M + H]⁺ Ethyl 5-(5-(6-amino-5- fluoropyridin-2-yl)-2- chlorobenzamido)-1-phenyl-1H- pyrazole-3-carboxylate (Example 149). |

### Example 143

### Ethyl 5-(5-(6-aminopyridin-2-yl)-2-chlorobenzamido)-1-phenyl-1H-pyrazole-3-carboxylate

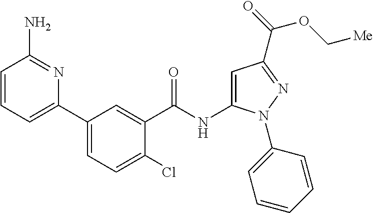 [SMILES: CCOC(=O)c1cc(NC(=O)c2cc(-c3cccc(N)n3)ccc2Cl)n(-c2ccccc2)n1]

6-amino-2-bromopyridine (835.5 mg, 4.83 mmol), sodium carbonate (1.39 g, 13.2 mmol) and water (0.5 mL) were added to the solution of ethyl 5-(2-chloro-5-(4,4,5,5-tetramethyl-1,3,2-dioxaborolan-2-yl)benzamido)-1-phenyl-1H-pyrazole-3-carboxylate in dioxane obtained in Preparation 6. The reaction was degassed with nitrogen for 30 minutes before the addition of Pd(dppf)Cl₂ with further degassing for 10 minutes. The reaction was heated to 100° C. for 16 hours. The reaction was cooled and eluted through a pad of silica, eluting with ethyl acetate (50 mL). The organic solution was washed with water (50 mL) dried over magnesium sulfate and concentrated in vacuo. The residue was purified using silica gel column chromatography eluting with 30-50% EtOAc in heptanes to afford the title compound as a pale yellow solid (1.02 g, 51%).

¹H NMR (400 MHz, CDCl₃): δ ppm 1.42 (t, 3H), 4.44 (q, 2H), 4.92 (br s, 2H), 6.55 (d, 1H), 7.08 (d, 1H), 7.38-7.57 (m, 7H), 7.97 (d, 1H), 8.43 (s, 1H), 8.66 (br s, 1H).

LCMS Rt=2.33 minutes MS m/z 462 [M+H]⁺

### Example 144

### Ethyl 5-(5-(6-amino-3,5-difluoropyridin-2-yl)-2-chlorobenzamido)-1-phenyl-1H-pyrazole-3-carboxylate

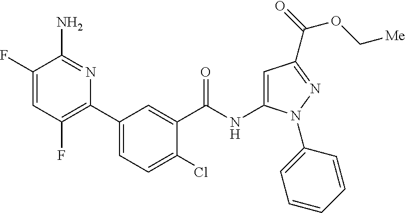 [SMILES: CCOC(=O)c1cc(NC(=O)c2cc(-c3nc(N)c(F)cc3F)ccc2Cl)n(-c2ccccc2)n1]

A solution of ethyl 5-(2-chloro-5-(4,4,5,5-tetramethyl-1,3,2-dioxaborolan-2-yl)benzamido)-1-phenyl-1H-pyrazole-3-carboxylate (Preparation 6, 1.5 g, 3.03 mmol), 6-bromo-3,5-difluoropyridin-2-amine (695 mg, 3.33 mmol), tetrakis(triphenylphosphine)palladium (350 mg, 0.30 mmol) and potassium carbonate (836 mg, 6.05 mmol) in dioxane (20 mL) and water (5 mL) was heated to 110° C. for 2 hours. The reaction was cooled to room temperature and partitioned between water (100 mL) and ethyl acetate (2×100 mL). The organic layer was washed with brine (100 mL), dried over magnesium sulphate and concentrated in vacuo. The solid residue was triturated with TBME and heptanes to afford the title compound as a white solid (1.10 g, 73%).

¹H NMR (400 MHz, DMSO-d₆): δ ppm 1.31 (t, 3H), 4.31 (q, 2H), 6.38 (s, 2H), 7.00 (s, 1H), 7.48-7.63 (m, 6H), 7.73 (t, 1H), 7.90 (s, 2H), 10.86 (s, 1H).

LCMS Rt=3.15 minutes MS m/z 498 [M+H]⁺

### Example 145

### Ethyl 5-(5-(6-aminopyridin-2-yl)-2,4-dichlorobenzamido)-1-phenyl-1H-pyrazole-3-carboxylate

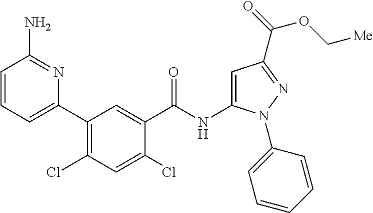 [SMILES: CCOC(=O)c1cc(NC(=O)c2cc(-c3cccc(N)n3)c(Cl)cc2Cl)n(-c2ccccc2)n1]

Ethyl 5-(2,4-dichloro-5-(4,4,5,5-tetramethyl-1,3,2-dioxaborolan-2 yl)benzamido)-1-phenyl-1H-pyrazole-3-carboxylate (Preparation 11, 94 mg, 0.21 mmol), 6-bromopyridine-2-amine (55 mg, 0.31 mmol) and potassium carbonate (58 mg, 0.42 mmol) were dissolved in dioxane (5 mL) and water (0.2 mL). The resulting solution was degassed by bubbling with nitrogen for 10 minutes before Pd(PPh₃)₄ (24 mg, 0.021 mmol) was added and the reaction was heated to reflux for 1.5 hours. The reaction was cooled and purified directly using silica gel column chromatography eluting with 20-70% EtOAc in heptanes to afford the title compound (43 mg, 41%).

¹H NMR (400 MHz, CDCl₃): δ ppm 1.36 (t, 3H), 4.34 (q, 2H), 4.67 (br s, 1H), 6.47 (d, 1H), 6.86 (d, 1H), 7.43-7.47 (m, 7H), 7.93 (s, 1H), 8.62 (br s, 1H).

### Example 146

### Ethyl 5-(5-(6-amino-3-cyano-5-fluoropyridin-2-yl)-2-chlorobenzamido)-1-phenyl-1H-pyrazole-3-carboxylate

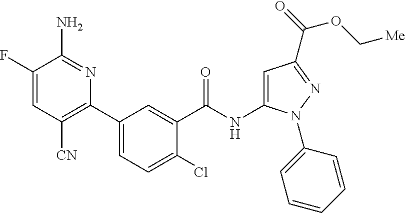 [SMILES: CCOC(=O)c1cc(NC(=O)c2cc(-c3nc(N)c(F)cc3C#N)ccc2Cl)n(-c2ccccc2)n1]

The title compound was prepared according to the method described for Example 143 using 6-amino-2-chloro-5-fluoronicotinonitrile (Preparation 30). The residue was purified using silica gel column chromatography eluting with 5% EtOAc in DCM.

¹H NMR (400 MHz, CDCl₃): δ ppm 1.39 (t, 3H), 4.40 (q, 2H), 5.68 (s, 2H), 7.32 (s, 1H), 7.41-7.54 (m, 7H), 7.86 (dd, 1H), 8.19 (d, 1H), 8.65 (s, 1H).

LCMS Rt=1.59 minutes MS m/z 505 [M+H]⁺

The following Examples were prepared according to the Method described by Example 143 or 144 using ethyl 5-(2-chloro-5-(4,4,5,5-tetramethyl-1,3,2-dioxaborolan-2-yl)benzamido)-1-phenyl-1H-pyrazole-3-carboxylate (Preparation 6) and the appropriate halopyridine as described below.

| Ex | | | |
|---|---|---|---|
| Number | Name | Structure | Data/Halopyridine |
| 147 | Ethyl 5-(5-(6-amino-3- fluoropyridin-2-yl)-2- chlorobenzamido)-1- phenyl-1H-pyrazole-3- carboxylate | 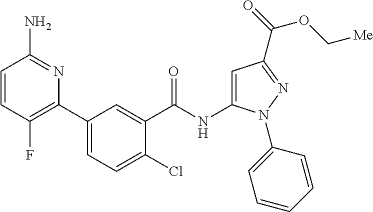 [SMILES: CCOC(=O)c1cc(NC(=O)c2cc(-c3nc(N)ccc3F)ccc2Cl)n(-c2ccccc2)n1] | ¹H NMR (400 MHz, DMSO-d₆): δ ppm 1.31 (t, 3H), 4.31 (q, 2H), 6.05 (s, 2H), 6.50 (dd, 1H), 7.00 (s, 1H), 7.43-7.62 (m, 6H), 7.92-7.97 (m, 2H), 10.85 (s, 1H). LCMS Rt = 3.02 minutes MS m/z 480 [M + H]⁺ 6-bromo-5-fluoropyridin-2- amine |
| 148 | Ethyl 5-(5-(6-amino-3- chloropyridin-2-yl)-2- chlorobenzamido)-1- phenyl-1H-pyrazole-3- carboxylate | 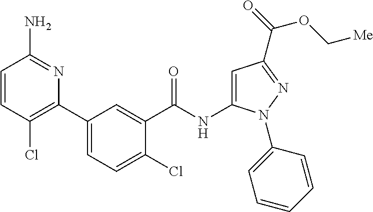 [SMILES: CCOC(=O)c1cc(NC(=O)c2cc(-c3nc(N)ccc3Cl)ccc2Cl)n(-c2ccccc2)n1] | ¹H NMR (400 MHz, MeOH-d₄): δ ppm 1.40 (t, 3H), 4.40 (q, 2H), 6.57 (d, 1H), 7.07 (s, 2H), 7.50-7.60 (m, 7H), 7.71-7.75 (m, 2H). LCMS Rt = 2.98 minutes MS m/z 496 [M + H]⁺ 6-bromo-5-chloropyridin-2- amine. |
| 149 | Ethyl 5-(5-(6-amino-5- fluoropyridin-2-yl)-2- chlorobenzamido)-1- phenyl-1H-pyrazole-3- carboxylate | 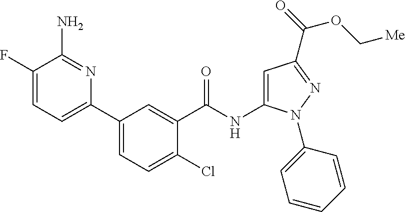 [SMILES: CCOC(=O)c1cc(NC(=O)c2cc(-c3ccc(F)c(N)n3)ccc2Cl)n(-c2ccccc2)n1] | ¹H NMR (400 MHz, MeOH-d₄): δ ppm 1.34 (t, 3H), 4.32 (q, 2H), 7.08 (s, 1H), 7.34 (t, 1H), 7.48-7.57 (m, 7H), 7.98 (d, 1H), 8.02 (s, 1H). LCMS Rt = 3.25 minutes MS m/z 480 [M + H]⁺ 6-bromo-3-fluoropyridin-2- amine. |

### Example 150

### 5-(6-amino-3-chloro-5-fluoropyridin-2-yl)-2-chloro-N-(3-cyano-1-phenyl-1H-pyrazol-5-yl)benzamide

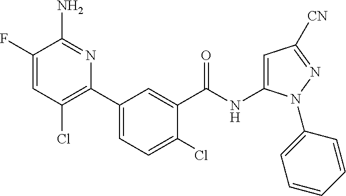 [SMILES: N#Cc1cc(NC(=O)c2cc(-c3nc(N)c(F)cc3Cl)ccc2Cl)n(-c2ccccc2)n1]

To a solution of 5-(6-((tert-butoxycarbonyl)amino)-3-chloro-5-fluoropyridin-2-yl)-2-chlorobenzoic acid (Preparation 23, 400 mg, 0.997 mmol), 5-amino-1-phenyl-1H-pyrazole-3-carbonitrile (Preparation 21, 193 mg, 1.05 mmol) and pyridine (321 μL, 3.99 mmol) in 2-methyltetrahydrofuran (6 mL) at 85° C. was added dropwise propylphosphonic anhydride (50% solution in ethyl acetate; 1.08 mL, 1.69 mmol). After stirring at 85° C. for 16 hours the reaction was cooled to room temperature. 2M NaOH (aq) solution (10 mL) and ethyl acetate (10 mL) were added, and the resulting mixture partitioned. The aqueous layer was extracted with ethyl acetate (2×10 mL), and the combined organic layers washed with brine (10 mL), dried over MgSO₄, and concentrated in vacuo. The residue was purified using silica gel column chromatography eluting with 1% methanol in dichloromethane followed by elution through an SCX-2 column using MeOH followed by 7N NH₃ in MeOH to afford the title compound (25 mg, 5%).

¹H NMR (400 MHz, CDCl₃): δ ppm 4.70 (m, 2H), 7.28-7.60 (m, 8H), 7.78 (dd, 1H), 8.23 (s, 1H), 8.57 (br s, 1H).

LCMS Rt=3.38 minutes MS m/z 467 [M+H]⁺

### Example 151

### 5-(6-aminopyridin-2-yl)-2-chloro-N-(3-cyano-1-phenyl-1H-pyrazol-5-yl)benzamide

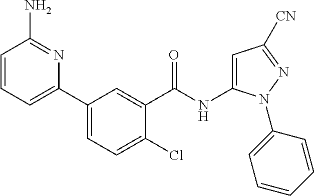 [SMILES: N#Cc1cc(NC(=O)c2cc(-c3cccc(N)n3)ccc2Cl)n(-c2ccccc2)n1]

To a solution of 2-chloro-N-(3-cyano-1-phenyl-1H-pyrazol-5-yl)-5-(4,4,5,5-tetramethyl-1,3,2-dioxaborolan-2-yl)benzamide (Preparation 8, 200 mg, 0.50 mmol) in dioxane (5 mL) was added 2-amino-6-bromopyridine (95 mg, 0.55 mmol), sodium carbonate (185 mg, 1.75 mmol) and water (1 mL). Nitrogen was bubbled through the reaction for 15 minutes followed by the addition of [1,1′-bis(diphenylphosphino)ferrocene]dichloropalladium(II) (41 mg, 0.05 mmol) and the reaction was heated to 100° C. for 12 hours. The reaction was diluted with ethyl acetate (100 mL), filtered through celite and washed with brine (250 mL). The organic layer was dried over magnesium sulphate and concentrated in vacuo. The residue was purified using silica gel column chromatography eluting with 0-50% ethyl acetate in dichloromethane to afford the title compound (145 mg, 70%).

¹H NMR (400 MHz, DMSO-d₆): δ ppm 6.05 (s, 2H), 6.42 (d, 1H), 7.08 (d, 1H), 7.30 (s, 1H), 7.4 to 7.7 (m, 7H), 8.04 (m, 2H), 11.01 (s, 1H).

MS m/z 415 [M+H]⁺

### Example 152

### 5-(6-amino-3,5-difluoropyridin-2-yl)-2-chloro-N-(3-cyano-1-phenyl-1H-pyrazol-5-yl)benzamide

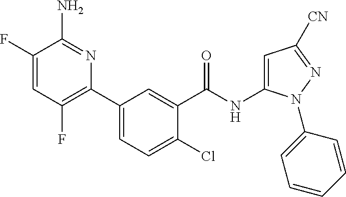 [SMILES: N#Cc1cc(NC(=O)c2cc(-c3nc(N)c(F)cc3F)ccc2Cl)n(-c2ccccc2)n1]

To solution of 2-chloro-N-(3-cyano-1-phenyl-1H-pyrazol-5-yl)-5-(4,4,5,5-tetramethyl-1,3,2-dioxaborolan-2-yl)benzamide (Preparation 8, 200 mg, 0.445 mmol) in dioxane (5 mL) was added 6-bromo-3,5-difluoropyridin-2-amine (103 mg, 0.49 mmol), potassium carbonate (123 mg, 1.1 mmol) and water (1 mL). The reaction was degassed with nitrogen before the addition of tetrakis(triphenylphosphine)palladium(0) (51.5 mg, 0.044 mmol). The reaction was heated to 100° C. for 90 minutes before cooling and concentrating in vacuo. The residue was purified using silica gel column chromatography eluting with 40% EtOAc in heptanes to afford the title compound (110 mg, 55%).

¹H NMR (400 MHz, DMSO-d₆): δ ppm 6.40 (s, 2H), 7.30 (s, 1H), 7.65-7.53 (m, 6H), 7.78 (dd, 1H), 7.90 (s, 1H), 7.93 (s, 1H), 11.03 (s, 1H).

LCMS Rt=3.19 minutes MS m/z 451 [M+H]⁺

### Example 153

### 5-{[5-(6-aminopyridin-2-yl)-2-chloro-4-fluorobenzoyl]amino}-1-phenyl-N-(1H-1,2,3-triazol-5-ylmethyl)-1H-pyrazole-3-carboxamide

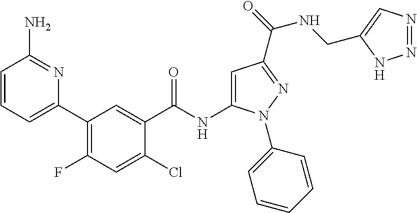 [SMILES: Nc1cccc(-c2cc(C(=O)Nc3cc(C(=O)NCc4cnn[nH]4)nn3-c3ccccc3)c(Cl)cc2F)n1]

To a solution of 5-(5-(6-aminopyridin-2-yl)-2-chloro-4-fluorobenzamido)-1-phenyl-1H-pyrazole-3-carboxylic acid (Preparation 35, 100 mg, 0.220 mmol), (1H-1,2,3-triazol-5-yl)methanamine hydrochloride (44 mg, 0.33 mmol) and DIPEA (114 mg, 0.88 mmol) in DMF (2 mL) was added HATU (126 mg, 0.33 mmol) and the mixture stirred at room temperature for 17 hours. Aqueous sodium hydroxide solution (2M, 0.5 mL) was then added and the mixture stirred for 0.5 hour. The mixture was purified by reverse phase column chromatography using basic conditions to give a colourless solid (45 mg). The solid was dissolved in methanol (20 mL), diluted with water (5 mL) and concentrated in vacuo to low volume (˜6 mL). The resulting solid was filtered to give the title compound as a colourless solid (10 mg, 9%).

¹H NMR (400 MHz, MeOH-d₄): δ ppm 4.69 (s, 2H), 6.60 (d, 1H), 7.00 (dd, 1H), 7.04 (s, 1H), 7.40 (d, 1H), 7.45-7.58 (m, 5H), 7.60 (d, 2H), 8.00 (d, 1H).

LCMS Rt=2.53 minutes, m/z=532.12 [M+H]⁺

### Example 154

### 5-{[5-(6-aminopyridin-2-yl)-2,4-dichlorobenzoyl]amino}-1-phenyl-N-(1H-1,2,3-triazol-5-ylmethyl)-1H-pyrazole-3-carboxamide

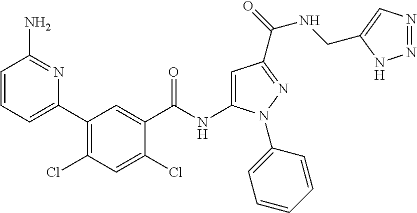 [SMILES: Nc1cccc(-c2cc(C(=O)Nc3cc(C(=O)NCc4cnn[nH]4)nn3-c3ccccc3)c(Cl)cc2Cl)n1]

To a suspension of 5-(5-(6-aminopyridin-2-yl)-2,4-dichlorobenzamido)-1-phenyl-1H-pyrazole-3-carboxylic acid (Preparation 36, 115 mg, 0.25 mmol), (1H-1,2,3-triazol-5-yl)methanamine hydrochloride (34 mg, 0.25 mmol) and DIPEA (97 mg, 0.75 mmol) in DMF (2 mL) was added HATU (95 mg, 0.25 mmol) and the mixture stirred at room temperature for 18 hours. Ethyl acetate (40 mL) and water (40 mL) were then added and the organic layer separated. The organic layer was washed with water (20 mL), saturated brine solution (10 mL), dried over magnesium sulphate and evaporated to dryness. The residue was purified by reverse phase column chromatography using basic conditions to give a colourless solid (42 mg, 30%).

¹H NMR (400 MHz, DMSO-d₆): δ ppm 4.50 (s, 2H), 6.10 (s, 2H), 6.48 (d, 1H), 6.75 (d, 1H), 6.92 (s, 1H), 7.45-7.62 (m, 9H), 7.79 (s, 1H), 8.80 (br s, 1H), 10.80 (br s, 1H).

LCMS Rt=2.63 minutes, m/z=548.28 [M+H]⁺

### Example 155

### 5-{[5-(6-aminopyridin-2-yl)-2,4-dichlorobenzoyl]amino}-N-[(3-hydroxyoxetan-3-yl)methyl]-1-phenyl-1H-pyrazole-3-carboxamide

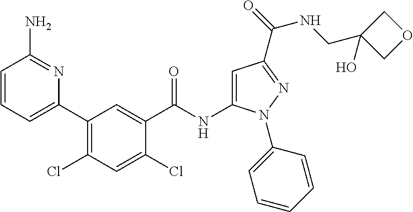 [SMILES: Nc1cccc(-c2cc(C(=O)Nc3cc(C(=O)NCC4(O)COC4)nn3-c3ccccc3)c(Cl)cc2Cl)n1]

The title compound was prepared according to the method described for Example 154 using 5-(5-(6-aminopyridin-2-yl)-2,4-dichlorobenzamido)-1-phenyl-1H-pyrazole-3-carboxylic acid (Preparation 36) and 3-(aminomethyl)oxetan-3-ol.

¹H NMR (400 MHz, DMSO-d₆): δ ppm 3.58 (s, 2H), 4.38 (d, 2H), 4.47 (d, 2H), 5.90 (s, 1H), 6.12 (s, 2H), 6.47 (d, 1H), 6.75 (d, 1H), 6.92 (s, 1H), 7.45-7.62 (m, 7H), 7.79 (s, 1H), 8.27 (br s, 1H), 10.81 (br s, 1H).

LCMS Rt=2.44 minutes, m/z=553.17 [M+H]⁺

### Example 156

### 5-{[5-(6-aminopyridin-2-yl)-2,4-dichlorobenzoyl]amino}-1-phenyl-N-(1H-pyrazol-4-ylmethyl)-1H-pyrazole-3-carboxamide

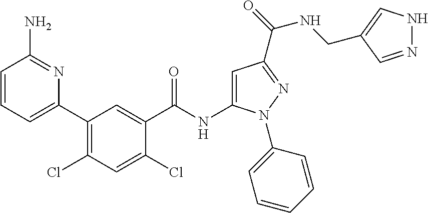 [SMILES: Nc1cccc(-c2cc(C(=O)Nc3cc(C(=O)NCc4cn[nH]c4)nn3-c3ccccc3)c(Cl)cc2Cl)n1]

The title compound was prepared according to the method described for Example 154 using 5-(5-(6-aminopyridin-2-yl)-2,4-dichlorobenzamido)-1-phenyl-1H-pyrazole-3-carboxylic acid (Preparation 36) and (1H-pyrazol-4-yl)methanamine hydrochloride. The crude residue was purified using an SCX-2 column, followed by recrystallization from methanol:water (1:9). The resulting solid was further purified using silica gel chromatography, eluting with 3-10% methanol in dichloromethane, followed by recrystallization from methanol:water (1:9) to give a colourless solid.

¹H NMR (400 MHz, MeOH-d₄): δ ppm 4.45 (s, 2H), 6.61 (d, 1H), 6.80 (d, 1H), 7.00 (s, 1H), 7.42-7.70 (m, 10H).

LCMS Rt=2.41 minutes, m/z=548.22 [M+H]⁺

### Example 157

### 5-{[5-(6-aminopyridin-2-yl)-2,4-dichlorobenzoyl]amino}-N-(3-methyl-1H-pyrazol-4-yl)-1-phenyl-1H-pyrazole-3-carboxamide

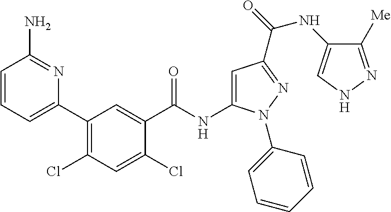 [SMILES: Cc1n[nH]cc1NC(=O)c1cc(NC(=O)c2cc(-c3cccc(N)n3)c(Cl)cc2Cl)n(-c2ccccc2)n1]

To a solution of 5-(5-(6-((tert-butoxycarbonyl)amino)pyridin-2-yl)-2,4-dichlorobenzamido)-1-phenyl-1H-pyrazole-3-carboxylic acid (Preparation 37, 150 mg, 0.26 mmol), 4-amino-3-methyl pyrazole (38 mg, 0.39 mmol) and DIPEA (34 mg, 0.26 mmol) in DMF (3 mL) was added HATU (99 mg, 0.26 mmol) and the mixture stirred at room temperature for 2 hours. Ethyl acetate (50 mL) and water (50 mL) were then added and the organic layer separated. The organic layer was washed with water (25 mL), saturated aqueous ammonium chloride solution (25 mL), saturated aqueous sodium bicarbonate solution (25 mL), saturated brine solution (10 mL), dried over sodium sulphate and evaporated to dryness. The residue was purified by silica gel column chromatography using ethyl acetate, then crystallised from dichloromethane. The solid was then suspended in dichloromethane (0.5 mL) and treated with TFA (0.5 mL) and the solution stirred at room temperature for 18 hours. The mixture was concentrated in vacuo and azeotroped with dichloromethane (2×1 mL), and with methanol (2×1 mL). The crude residue was purified using an SCX-2 column to give a crystalline solid (70 mg, 48%).

¹H NMR (400 MHz, DMSO-d₆): δ ppm 2.17 (s, 3H), 6.09 (s, 2H), 6.48 (d, 1H), 6.75 (d, 1H), 6.98 (s, 1H), 7.45-7.62 (m, 8H), 7.79 (s, 1H), 9.45-9.55 (br s×2, 1H), 10.80 (s, 1H), 12.30-12.45 (br s×2, 1H); evidence of rotamers.

LCMS Rt=2.63 minutes, m/z=547.10 [M+H]⁺

The following Examples were made by methods analogous to those described above.

### Example 158: 5-(5-(6-aminopyridin-2-yl)-2-chlorobenzamido)-4-methyl-1-phenyl-1H-pyrazole-3-carboxamide

### Example 159: N-(4-amino-3-methyl-1-phenyl-1H-pyrazol-5-yl)-5-(6-aminopyridin-2-yl)-2-chlorobenzamide

### Preparation 1

### 5-(5-(6-((tert-butoxycarbonyl)amino)pyridin-2-yl)-2-chlorobenzamido)-1-phenyl-1H-pyrazole-3-carboxylic Acid

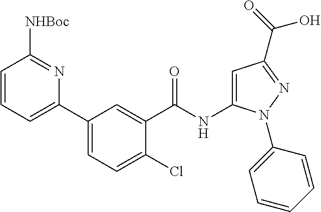 [SMILES: CC(C)(C)OC(=O)Nc1cccc(-c2ccc(Cl)c(C(=O)Nc3cc(C(=O)O)nn3-c3ccccc3)c2)n1]

The title compound was prepared according to the method described for Example 138 using ethyl 5-(5-(6-((tert-butoxycarbonyl)amino)pyridin-2-yl)-2-chlorobenzamido)-1-phenyl-1H-pyrazole-3-carboxylate (Preparation 3). The reaction was acidified with 2M HCl to precipitate a white solid that was filtered, dissolved in MeOH, dried over sodium sulphate and concentrated in vacuo.

¹H NMR (400 MHz, DMSO-d₆): δ ppm 1.46 (s, 9H), 6.75 (s, 1H), 7.41 (t, 1H), 7.53 (t, 2H), 7.00-7.68 (m, 4H), 7.75 (d, 1H), 7.85 (t, 1H), 8.16 (dd, 1H), 8.22 (d, 1H), 9.80 (s, 1H), 10.77 (br s, 1H).

The following Preparations were prepared according to the Method described by Example 143 or 144 using ethyl 5-(2-chloro-5-(4,4,5,5-tetramethyl-1,3,2-dioxaborolan-2-yl)benzamido)-1-phenyl-1H-pyrazole-3-carboxylate (Preparation 6) or 2-chloro-N-(3-cyano-1-phenyl-1H-pyrazol-5-yl)-5-(4,4,5,5-tetramethyl-1,3,2-dioxaborolan-2-yl)benzamide (Preparation 8) and the appropriate halopyridine as described below.

| Prep | | | |
|---|---|---|---|
| Number | Name | Structure | Data/Halopyridine |
| 2 | Ethyl 5-(5-(6-(tert- butylamino)-5- methylpyridin-2-yl)-2- chlorobenzamido)-1- phenyl-1H-pyrazole-3- carboxylate | 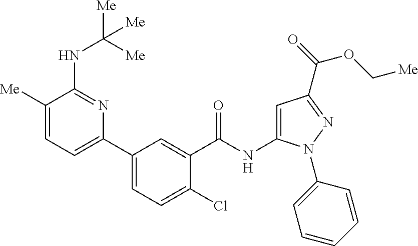 [SMILES: CCOC(=O)c1cc(NC(=O)c2cc(-c3ccc(C)c(NC(C)(C)C)n3)ccc2Cl)n(-c2ccccc2)n1] | ¹H NMR (400 MHz, CDCl₃) δ ppm 1.40 (m, 3H), 1.55 (s, 9H), 2.10 (s, 3H), 4.45 (m, 2H), 7.00 (m, 1H), 7.20-7.40 (m, 7H), 8.10 (m, 1H), 8.45 (m, 2H). 6-bromo-N-(tert-butyl)-3- methylpyridin-2-amine (WO2010138589). |
| 3 | Ethyl 5-(5-(6-((tert- butoxycarbonyl)amino) pyridin-2-yl)-2- chlorobenzamido)-1- phenyl-1H-pyrazole-3- carboxylate | 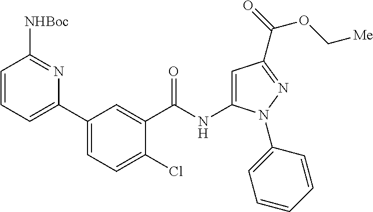 [SMILES: CCOC(=O)c1cc(NC(=O)c2cc(-c3cccc(NC(=O)OC(C)(C)C)n3)ccc2Cl)n(-c2ccccc2)n1] | Using tert-butyl (6- bromopyridin-2-yl)carbamate at 100° C. for 2 hours Taken on directly to the next step. |
| 4 | tert-butyl (3-chloro-6- (4-chloro-3-((3-cyano- 1-phenyl-1H-pyrazol- 5- yl)carbamoyl)phenyl) pyridin-2-yl)carbamate | 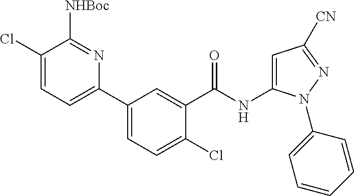 [SMILES: CC(C)(C)OC(=O)Nc1nc(-c2ccc(Cl)c(C(=O)Nc3cc(C#N)nn3-c3ccccc3)c2)ccc1Cl] | 1H NMR (400 MHz, DMSO-d6): δ ppm 1.46 (s, 9H), 7.30 (s, 1H), 7.61-7.58 (m, 7H), 7.91 (d, 1H), 8.09 (d, 1H), 8.19 (s, 1H), 9.53 (s, 1H), 11.08 (s, 1H). tert-butyl (3,6-dichloropyridin-2- yl)carbamate. |

### Preparation 5

### Ethyl 5-(5-(6-((tert-butoxycarbonyl)amino)pyridin-2-yl)-2,4-dichlorobenzamido)-1-phenyl-1H-pyrazole-3-carboxylate

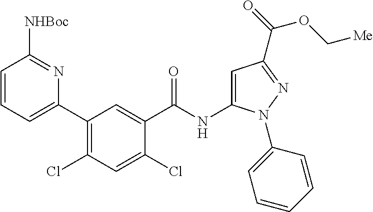 [SMILES: CCOC(=O)c1cc(NC(=O)c2cc(-c3cccc(NC(=O)OC(C)(C)C)n3)c(Cl)cc2Cl)n(-c2ccccc2)n1]

A solution of 5-(6-((tert-butoxycarbonyl)amino)pyridin-2-yl)-2,4-dichlorobenzoic acid (Preparation 24, 620 mg, 1.62 mmol), ethyl 5-amino-1-phenyl-1H-pyrazole-3-carboxylate (374 mg, 1.62 mmol) and DIPEA (0.847 mL, 4.86 mmol) in 2-methyltetrahydrofuran (15 mL) was heated under nitrogen to 85° C. To the solution was added T3P (50% in EtOAc; 2.90 mL, 4.86 mmol) dropwise over 5 minutes. The reaction was heated at 85° C. for 5 hours before cooling to room temperature and partitioning between saturated aqueous NaHCO₃ solution (30 mL) and EtOAc (20 mL). The aqueous layer was washed with EtOAc (20 mL), the organic layers combined, dried over sodium sulphate and concentrated in vacuo. The residue was purified using silica gel column chromatography eluting with 30-50% TBME in heptanes to afford the title compound as a white solid (750 mg, 77%).

¹H NMR (400 MHz, CDCl₃): δ ppm 1.41 (t, 3H), 1.56 (s, 9H), 4.45 (m, 2H), 7.01 (s, 1H), 7.30 (d, 1H), 7.40 (s, 1H), 7.50-7.62 (m, 5H), 7.73 (t, 1H), 7.97 (d, 1H), 8.14 (s, 1H), 8.42 (s, 1H).

### Preparation 6

### Ethyl 5-(2-chloro-5-(4,4,5,5-tetramethyl-1,3,2-dioxaborolan-2-yl)benzamido)-1-phenyl-1H-pyrazole-3-carboxylate

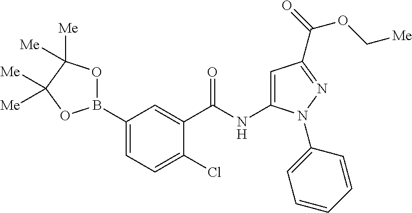 [SMILES: CCOC(=O)c1cc(NC(=O)c2cc(B3OC(C)(C)C(C)(C)O3)ccc2Cl)n(-c2ccccc2)n1]

To a degassed solution of ethyl 5-(5-bromo-2-chlorobenzamido)-1-phenyl-1H-pyrazole-3-carboxylate (Preparation 17, 1.97 g, 4.39 mmol) and bis-pinacolatodiboron (1.23 g, 4.83 mmol) in dioxane (20 mL) was added potassium acetate (1.29 g, 13.7 mmol) and palladium(diphenylphosphineferrocenyl)dichloride (179 mg, 0.22 mmol). The reaction was degassed for a further 30 minutes before heating to 100° C. for 16 hours. The reaction was cooled to room temperature and used directly in the next step.

### Preparation 7

### N-(2-((tert-butyldimethylsilyl)oxy)ethyl)-5-(2-chloro-5-(4,4,5,5-tetramethyl-1,3,2-dioxaborolan-2-yl)benzamido)-1-phenyl-1H-pyrazole-3-carboxamide

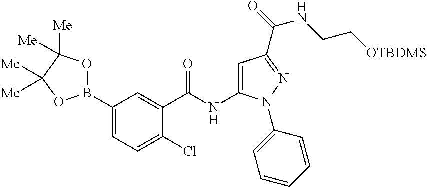 [SMILES: CC1(C)OB(c2ccc(Cl)c(C(=O)Nc3cc(C(=O)NCCO[Si](C)(C)C(C)(C)C)nn3-c3ccccc3)c2)OC1(C)C]

To a solution of 5-(5-bromo-2-chlorobenzamido)-N-(2-((tert-butyldimethylsilyl)oxy)ethyl)-1-phenyl-1H-pyrazole-3-carboxamide (Preparation 13, 3.56 g, 6.16 mmol), bis(pinacolato)diboron (2.35 g, 9.24 mmol) and potassium acetate (1.21 g, 12.32 mmol) in dioxane (100 mL) was degassed with nitrogen for 10 minutes before the addition of Pd(dppf)Cl₂ (243 g, 0.31 mmol). The reaction was heated to reflux for 4 hours. The reaction was filtered through a pad of silica eluting with 20% EtOAc in heptanes to afford the title compound (3.70 g, 95%).

¹H NMR (400 MHz, CDCl₃): δ ppm 0.05 (s, 6H), 0.86 (s, 9H), 1.24 (s, 12H), 3.55 (q, 2H), 3.76 (t, 2H), 7.45-7.53 (m, 6H), 7.78 (dd, 1H), 8.12 (s, 1H), 8.21 (s, 1H).

### Preparation 8

### 2-chloro-N-(3-cyano-1-phenyl-1H-pyrazol-5-yl)-5-(4,4,5,5-tetramethyl-1,3,2-dioxaborolan-2-yl)benzamide

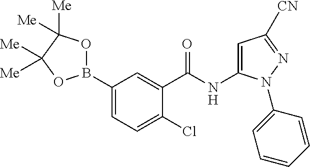 [SMILES: CC1(C)OB(c2ccc(Cl)c(C(=O)Nc3cc(C#N)nn3-c3ccccc3)c2)OC1(C)C]

The title compound was prepared according to the method described for Preparation 6 using 5-bromo-2-chloro-N-(3-cyano-1-phenyl-1H-pyrazol-5-yl)benzamide (Preparation 18). Taken on directly to the next step.

### Preparation 9

### 1-(3-(benzyloxy)phenyl)-5-(2-chloro-5-(4,4,5,5-tetramethyl-1,3,2-dioxaborolan-2-yl)benzamido)-1H-pyrazole-3-carboxamide

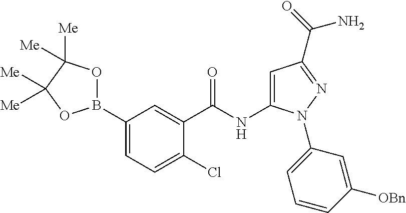 [SMILES: CC1(C)OB(c2ccc(Cl)c(C(=O)Nc3cc(C(N)=O)nn3-c3cccc(OCc4ccccc4)c3)c2)OC1(C)C]

The title compound was prepared according to the method described for Preparation 6 using 1-(3-(benzyloxy)phenyl)-5-(5-bromo-2-chlorobenzamido)-1H-pyrazole-3-carboxamide (Preparation 19).

¹H NMR (400 MHz, MeOH-d₄): δ ppm 1.31 (s, 12H), 5.12 (s, 2H), 7.00 (s, 1H), 7.11 (dd, 1H), 7.21 (dd, 1H), 7.27-7.36 (m, 4H), 7.41-7.49 (m, 4H), 7.78 (dd, 1H), 7.82 (s, 1H).

### Preparation 10

### 5-(2-chloro-5-(4,4,5,5-tetramethyl-1,3,2-dioxaborolan-2-yl)benzamido)-1-phenyl-1H-Pyrazole-3-carboxamide

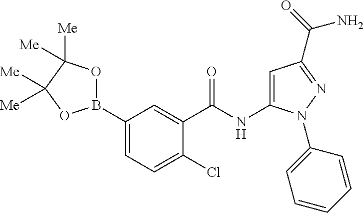 [SMILES: CC1(C)OB(c2ccc(Cl)c(C(=O)Nc3cc(C(N)=O)nn3-c3ccccc3)c2)OC1(C)C]

To solution of 5-(5-bromo-2-chlorobenzamido)-1-phenyl-1H-pyrazole-3-carboxamide (Preparation 15, 536 mg, 1.28 mmol) in dioxane (15 mL) was added 4,4,4′,4′,5,5,5′,5′-octamethyl-2,2′-bi(1,3,2-dioxaborolane) (390 mg, 1.54 mmol) and potassium acetate (380 mg, 3.85 mmol). The reaction was degassed with nitrogen for 5 minutes before the addition of Pd(dppf)Cl₂ (52 mg, 0.06 mmol) and heated to 100° C. for 2.5 hours. The reaction was cooled, filtered and the filtrate purified using silica gel column chromatography eluting with 70% EtOAc in heptanes to afford the title compound (463 mg, 60%).

¹H NMR (400 MHz, DMSO-d₆): δ ppm 1.15 (s, 12H), 6.89 (s, 1H), 7.37 (br s, 1H), 7.48-7.61 (m, 7H), 7.67 (br s, 1H), 7.71 (s, 1H), 10.66 (br s, 1H).

### Preparation 11

### Ethyl 5-(2,4-dichloro-5-(4,4,5,5-tetramethyl-1,3,2-dioxaborolan-2 yl)benzamido)-1-phenyl-1H-pyrazole-3-carboxylate

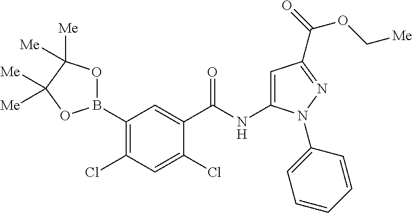 [SMILES: CCOC(=O)c1cc(NC(=O)c2cc(B3OC(C)(C)C(C)(C)O3)c(Cl)cc2Cl)n(-c2ccccc2)n1]

Ethyl 5-(5-bromo-2,4-dichlorobenzamido)-1-phenyl-1H-pyrazole-3-carboxylate (Preparation 16, 140 mg, 0.29 mmol), bis(pinacolato)diboron (110 mg, 0.43 mmol) and potassium acetate (57 mg, 0.58 mmol) were dissolved in dioxane (5 mL) and the resulting solution was degassed with nitrogen for 10 minutes. Pd(dppf)Cl₂ (22 mg, 0.028 mmol) was added and the reaction was heated at 110° C. for 3.5 hours. The reaction was cooled and purified directly using silica gel column chromatography eluting with 50% EtOAc in heptanes to afford the title compound (94 mg, 73%).

¹H NMR (400 MHz, CDCl₃): 1.32 (s, 12H), 1.39 (t, 3H), 4.41 (q, 2H), 7.35-7.36 (m, 2H), 7.47-7.51 (m, 5H), 8.17 (s, 1H), 8.33 (s, 1H).

### Preparation 12

### 5-(5-bromo-2-chlorobenzamido)-N-cyclopropyl-1-phenyl-1H-pyrazole-3-carboxamide

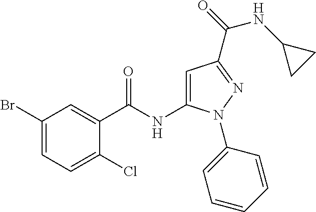 [SMILES: O=C(NC1CC1)c1cc(NC(=O)c2cc(Br)ccc2Cl)n(-c2ccccc2)n1]

To a solution of HATU (1.55 mmol, 588 mg) and 5-(5-bromo-2-chlorobenzamido)-1-phenyl-1H-pyrazole-3-carboxylic acid (Preparation 14, 500 mg, 1.19 mmol) in DMF (5 mL) was added DIPEA (1.04 mL, 5.94 mmol). The reaction was stirred at room temperature for 15 minutes before the addition of cyclopropylamine (1.08 mL, 1.55 mmol). The reaction was stirred at room temperature for 2 hours. The reaction was partitioned between EtOAc (20 mL) and saturated aqueous NaHCO₃ solution (30 mL). The organic layer was washed with 10% aqueous citric acid solution (30 mL), brine (30 mL), dried over sodium sulphate and concentrated in vacuo to afford the title compound (496 mg, 91%).

¹H NMR (400 MHz, DMSO-d₆): δ ppm 0.59-0.63 (m, 2H), 0.65-0.70 (m, 2H), 2.81-2.88 (m, 1H), 6.91 (s, 1H), 7.46-7.61 (m, 6H), 7.69-7.71 (dd, 1H), 7.73 (d, 1H), 8.30 (d, 1H), 10.79 (br s, 1H).

MS m/z 458 [M−H]⁻

### Preparation 13

### 5-(5-bromo-2-chlorobenzamido)-N-(2-((tert-butyldimethylsilyl)oxy)ethyl)-1-phenyl-1H-pyrazole-3-carboxamide

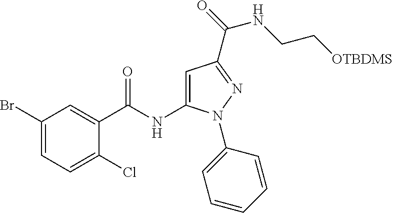 [SMILES: CC(C)(C)[Si](C)(C)OCCNC(=O)c1cc(NC(=O)c2cc(Br)ccc2Cl)n(-c2ccccc2)n1]

To a solution of 5-(5-bromo-2-chlorobenzamido)-1-phenyl-1H-pyrazole-3-carboxylic acid (Preparation 14, 6.76 g, 16.1 mmol), 2-((tert-butyldimethylsilyl)oxy)ethanamine (3.66 g, 20.9 mmol) and diisopropylethylamine (4.15 g, 5.72 mL, 32.1 mmol) in DMF (60 mL) was added HATU (7.94 g, 20.9 mmol) and the reaction was stirred at room temperature for 18 hours. The reaction was concentrated in vacuo and partitioned between ethyl acetate (50 mL) and water (50 mL). The aqueous phase was extracted with ethyl acetate (3×50 mL). The combined organic layers were dried over MgSO₄ and concentrated in vacuo. The residue was purified using silica gel column chromatography eluting with 30% ethyl acetate in heptanes to afford the title compound (3.66 g, 39%).

¹H NMR (400 MHz, CDCl₃): δ ppm 0.05 (s, 6H), 0.87 (s, 9H), 3.54 (q, 2H), 3.75 (t, 2H), 7.21-7.30 (m, 3H), 7.47-7.51 (m, 6H), 7.98 (s, 1H), 8.37 (s, 1H).

### Preparation 14

### 5-(5-bromo-2-chlorobenzamido)-1-phenyl-1H-pyrazole-3-carboxylic Acid

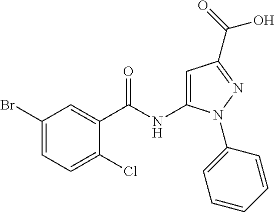 [SMILES: O=C(O)c1cc(NC(=O)c2cc(Br)ccc2Cl)n(-c2ccccc2)n1]

To a suspension of ethyl 5-(5-bromo-2-chlorobenzamido)-1-phenyl-1H-pyrazole-3-carboxylate (Preparation 17, 2.81 mmol, 1.26 g) in EtOH (14 mL) was added 0.5M aqueous LiOH solution (14.04 mmol, 336 mg in 28 mL of water). The reaction was stirred at 40° C. for 3 hours before acidifying to pH=5 with 1N HCl. The resulting precipitate was filtered and dried to afford the title compound (1.13 g, 97%).

¹H NMR (400 MHz, DMSO-d₆): δ ppm 6.95 (s, 1H), 7.46-7.50 (m, 1H), 7.53-7.59 (m, 4H), 7.67-7.70 (dd, 1H), 7.73 (d, 1H), 9.80 (s, 1H), 10.84 (s, 1H), 13.02 (br s, 1H).

MS m/z 419 [M−H]⁻

### Preparation 15

### 5-(5-bromo-2-chlorobenzamido)-1-phenyl-1H-pyrazole-3-carboxamide

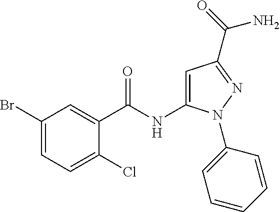 [SMILES: NC(=O)c1cc(NC(=O)c2cc(Br)ccc2Cl)n(-c2ccccc2)n1]

To a solution of 5-bromo-2-chloro-N-(3-cyano-1-phenyl-1H-pyrazol-5-yl)benzamide (Preparation 18, 500 mg, 1.24 mmol) in DMSO (5 mL) was added K₂CO₃ (340 mg, 2.48 mmol) followed by H₂O₂ (50% by weight, aqueous solution, 1.69 mL, 24.9 mmol). The reaction was stirred at room temperature for 3 hours. The reaction was quenched by the addition of saturated aqueous KHSO₄ solution (5 mL) followed by water (15 mL) and EtOAc (40 mL). The mixture was filtered and separated. The organic layer was washed with brine (40 mL), dried over MgSO₄, and concentrated in vacuo. The residue was slurried in water and filtered to afford the title compound (457 mg, 88%).

¹H NMR (400 MHz, DMSO-d₆): δ ppm 6.89 (s, 1H), 7.35 (br s, 1H), 7.44-7.72 (m, 9H), 10.77 (br s, 1H).

LCMS Rt=2.54 minutes MS m/z 419 [M−H]⁻

### Preparation 16

### Ethyl 5-(5-bromo-2,4-dichlorobenzamido)-1-phenyl-1H-pyrazole-3-carboxylate

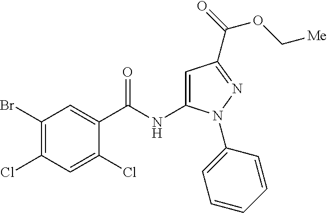 [SMILES: CCOC(=O)c1cc(NC(=O)c2cc(Br)c(Cl)cc2Cl)n(-c2ccccc2)n1]

5-Bromo-2,4-dichlorobenzoic acid (513 mg, 1.91 mmol), ethyl 5-amino-1-phenyl-1H-pyrazole-3-carboxylate (400 mg, 1.73 mmol) and pyridine (557 μL, 6.92 mmol) were dissolved in 2-methyltetrahydrofuran (20 mL) and the reaction was heated to reflux. Propylphosphonic anhydride (50% w/w solution in EtOAc, 1.65 mL, 2.59 mmol) was added and the reaction was continued heating at reflux for 18 hours. The reaction was cooled and washed with 2M aqueous HCl (30 mL), saturated aqueous NaHCO₃ solution (30 mL), brine (30 mL), dried over MgSO₄ and concentrated in vacuo. The residue was purified by silica gel column chromatography eluting with 40% EtOAc in heptanes to afford the title compound (326 mg, 39%).

¹H NMR (400 MHz, CDCl₃): δ ppm 1.40 (t, 3H), 4.43 (q, 2H), 7.35 (s, 1H), 7.46 (s, 1H), 7.51-7.55 (m, 5H), 8.16 (s, 1H), 8.45 (br s, 1H).

### Preparation 17

### Ethyl 5-(5-bromo-2-chlorobenzamido)-1-phenyl-1H-pyrazole-3-carboxylate

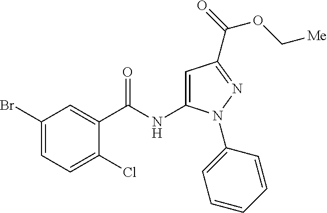 [SMILES: CCOC(=O)c1cc(NC(=O)c2cc(Br)ccc2Cl)n(-c2ccccc2)n1]

Pyridine (12.9 mL, 161 mmol) was added to a solution of ethyl 5-amino-1-phenyl-1H-pyrazole-3-carboxylate (9.32 g, 40.3 mmol) and 5-bromo-2-chlorobenzoic acid (10.4 g, 44.3 mmol) in 2-methyl-tetrahydrofuran (100 mL). The reaction was heated to 85° C. before the addition of propylphosphonic anhydride (38.5 mL, 60.4 mmol, 50% solution in ethyl acetate) drop-wise. The reaction was heated at this temperature for 16 hours before cooling to room temperature. The organic solution was washed with saturated aqueous sodium hydrogen carbonate solution (3×25 mL), saturated brine (30 mL) and concentrated in vacuo. The resulting solid was triturated with TBME (5×50 mL) to afford the title compound (13.7 g, 76%).

¹H NMR (400 MHz, CDCl₃): δ ppm 1.42 (t, 3H), 4.44 (q, 2H), 7.25 (d, 1H), 7.39 (s, 1H), 7.51 (m, 5H), 8.02 (d, 1H), 8.43 (s, 1H).

LCMS Rt=3.13 minutes MS m/z 448 [M+H]⁺

### Preparation 18

### 5-bromo-2-chloro-N-(3-cyano-1-phenyl-1H-pyrazol-5-yl)benzamide

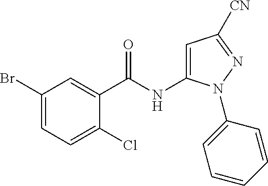 [SMILES: N#Cc1cc(NC(=O)c2cc(Br)ccc2Cl)n(-c2ccccc2)n1]

To a solution of 5-amino-1-phenyl-1H-pyrazole-3-carbonitrile (Preparation 21, 400 mg, 2.17 mmol) in 2-methyltetrahydrofuran (12 mL) was added pyridine (524 uL, 6.51 mmol) and 5-bromo-2-chlorobenzoic acid (768 mg, 3.26 mmol). The mixture was heated to 85° C. and 2,4,6-tripropyl-1,3,5,2,4,6-trioxatriphosphinane 2,4,6-trioxide (50% by weight solution in ethyl acetate, 3.10 mL, 4.34 mmol), was added dropwise. The reaction was heated at 85° C. for 16 hours, then stirred for 48 hours at room temperature. The reaction was quenched by the addition of 10% aqueous potassium carbonate solution (24 mL, 17.4 mmol) with stirring at room temperature for 1 hour. The reaction was diluted to 150 mL with ethyl acetate and washed with 10% aqueous solution of potassium carbonate (100 mL) followed by 2M aqueous HCl (100 mL). The organic layer was dried over magnesium sulphate and concentrated in vacuo. The residue was purified using silica gel column chromatography eluting with 0-1% methanol in dichloromethane to afford the title compound (640 mg, 74%).

¹H NMR (400 MHz, DMSO-d₆): δ ppm 7.24 (s, 1H), 7.45-7.62 (m, 6H), 7.72 (m, 2H), 11.04 (s, 1H).

### Preparation 19

### 1-(3-(benzyloxy)phenyl)-5-(5-bromo-2-chlorobenzamido)-1H-pyrazole-3-carboxamide

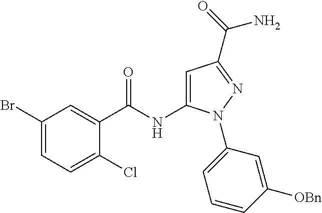 [SMILES: NC(=O)c1cc(NC(=O)c2cc(Br)ccc2Cl)n(-c2cccc(OCc3ccccc3)c2)n1]

Hydrogen peroxide (30% solution in water; 1.83 mL, 17.8 mmol) was added dropwise to a solution of N-(1-(3-(benzyloxy)phenyl)-3-cyano-1H-pyrazol-5-yl)-5-bromo-2-chlorobenzamide (Preparation 20, 453 mg, 0.89 mmol) and potassium carbonate (247 mg, 1.78 mmol) in dimethylsulfoxide (20 mL). The reaction mixture was stirred at room temperature for 22 hours then diluted with ethyl acetate (20 mL), washed with water (3×40 mL), dried over MgSO₄ and concentrated in vacuo. The residue was purified using reverse phase column chromatography eluting with 5-95% acetonitrile in water (with 0.1% formic acid) to afford the title compound as a colourless solid (413 mg, 88% yield).

¹H NMR (400 MHz, DMSO-d₆): δ ppm 5.14 (s, 2H), 6.89 (s, 1H), 7.11 (d, 1H), 7.20 (d, 1H), 7.29-7.50 (m, 9H), 7.66-7.70 (m, 2H), 7.74 (d, 1H), 10.80 (s, 1H).

### Preparation 20

### N-(1-(3-(benzyloxy)phenyl)-3-cyano-1H-pyrazol-5-yl)-5-bromo-2-chlorobenzamide

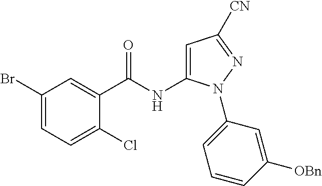 [SMILES: N#Cc1cc(NC(=O)c2cc(Br)ccc2Cl)n(-c2cccc(OCc3ccccc3)c2)n1]

The title compound was prepared according to the method described for Preparation 18 using 5-amino-1-(3-(benzyloxy)phenyl)-1H-pyrazole-3-carbonitrile (Preparation 22). The residue was purified using silica gel column chromatography eluting with 5-40% EtOAc in heptanes followed by trituration with TBME.

¹H NMR (400 MHz, DMSO-d₆): δ ppm 5.14 (s, 2H), 7.15-7.19 (m, 2H), 7.29-7.50 (m, 9H), 7.69 (dd, 1H), 7.74 (d, 1H).

### Preparation 21

### 5-amino-1-phenyl-1H-pyrazole-3-carbonitrile

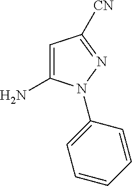 [SMILES: N#Cc1cc(N)n(-c2ccccc2)n1]

To a solution of 2-(phenyldiazenyl)succinonitrile (Preparation 29, 2.84 g, 15.43 mmol) in dichloromethane (103 mL) was added a 10% aqueous solution of potassium carbonate (77 mL, 55.71 mmol). The reaction was stirred at room temperature for 18 hours. The organic layer was separated dried over magnesium sulphate and concentrated in vacuo. The residue was purified using silica gel column chromatography eluting with 0-3% MeOH in DCM to afford the title compound (1.24 g, 44%).

¹H NMR (400 MHz, CDCl₃): δ ppm 3.96 (br s, 2H), 6.00 (s, 1H), 7.45 (m, 1H), 7.56 (m, 4H).

MS m/z 185 [M+H]⁺

### Preparation 22

### 5-amino-1-(3-(benzyloxy)phenyl)-1H-pyrazole-3-carbonitrile

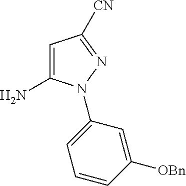 [SMILES: N#Cc1cc(N)n(-c2cccc(OCc3ccccc3)c2)n1]

Pyridine (1.10 mL, 13.7 mmol) was added to a solution of ethyldicyanopropionate (694 mg, 4.56 mmol) in methanol (25 mL). The solution was cooled to 0° C. and 3-(benzyloxy)benzenediazonium tetrafluoroborate (1.36 g, 4.56 mmol) was added portionwise over 40 minutes. The reaction was stirred at room temperature for 18 hours then concentrated in vacuo. The residue was dissolved in dichloromethane (50 mL) and a 10% aqueous solution of potassium carbonate (25 mL) was added. The biphasic mixture was stirred at room temperature for 3 hours. The organic layer collected, dried over MgSO₄ and concentrated in vacuo. The residue was purified using Biotage silica gel column chromatography eluting with 5-40% EtOAc in heptanes to afford the title compound as a dark red oil (640 mg, 48%).

¹H NMR (400 MHz, DMSO-d₆): δ ppm 3.94 (br s, 2H), 5.12 (s, 2H), 5.96 (s, 1H), 7.04-7.07 (m, 1H), 7.12-7.15 (m, 2H), 7.34-7.44 (m, 6H).

MS m/z 291 [M+H]⁺

### Preparation 23

### 5-(6-((tert-butoxycarbonyl)amino)-3-chloro-5-fluoropyridin-2-yl)-2-chlorobenzoic Acid

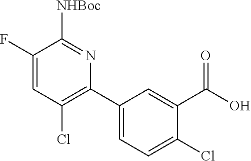 [SMILES: CC(C)(C)OC(=O)Nc1nc(-c2ccc(Cl)c(C(=O)O)c2)c(Cl)cc1F]

A mixture of methyl 5-(6-((tert-butoxycarbonyl)amino)-3-chloro-5-fluoropyridin-2-yl)-2-chlorobenzoate (Preparation 25, 250 mg, 0.602 mmol) and lithium hydroxide monohydrate (126 mg, 3.01 mmol) in water (2 mL) and tetrahydrofuran (5 mL) was stirred at room temperature for 16 hours. The reaction was concentrated in vacuo and diluted with water (3 mL). The solution was neutralised with 2M HCl (aq) and extracted into 2-methyltetrahydrofuran (3×5 mL). The combined organic layers were dried over MgSO₄ and concentrated in vacuo to afford the title compound as white solid (175 mg, 74%).

¹H NMR (400 MHz, MeOH-d₄): δ ppm 1.51 (s, 9H), 7.50 (d, 1H), 7.71 (dd, 1H), 7.85 (d, 1H), 8.01 (d, 1H).

### Preparation 24

### 5-(6-((tert-butoxycarbonyl)amino)pyridin-2-yl)-2,4-dichlorobenzoic Acid

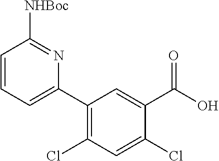 [SMILES: CC(C)(C)OC(=O)Nc1cccc(-c2cc(C(=O)O)c(Cl)cc2Cl)n1]

The title compound was prepared according to the method described for Preparation using methyl 5-(6-((tert-butoxycarbonyl)amino)pyridin-2-yl)-2,4-dichlorobenzoate (Preparation 28).

¹H NMR (400 MHz, CDCl₃): δ ppm 1.55 (s, 9H), 7.31 (d, 1H), 7.58 (s, 1H), 7.78 (t, 1H), 8.04 (d, 1H), 8.13 (s, 1H), 8.40 (br s, 1H).

### Preparation 25

### Methyl 5-(6-((tert-butoxycarbonyl)amino)-3-chloro-5-fluoropyridin-2-yl)-2-chlorobenzoate

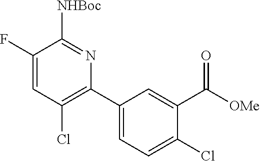 [SMILES: COC(=O)c1cc(-c2nc(NC(=O)OC(C)(C)C)c(F)cc2Cl)ccc1Cl]

To a mixture of methyl 5-(6-amino-3-chloro-5-fluoropyridin-2-yl)-2-chlorobenzoate (Preparation 26, 880 mg, 2.79 mmol), di-tert-butyldicarbonate (1.46 g, 6.70 mmol) and triethylamine (1.01 mL, 7.26 mmol) in dichloromethane was added 4-dimethylaminopyridine (136 mg, 1.12 mmol). The reaction was stirred at room temperature for 16 hours before partitioning between TBME and water. The aqueous layer was extracted with TBME (2×20 mL) and the combined organic layers were washed with brine (50 mL), dried over MgSO₄ and concentrated in vacuo. The residue was purified using silica gel column chromatography eluting with 10% ethyl acetate in heptanes to afford the title compound (541 mg, 47%).

¹H NMR (400 MHz, CDCl₃): δ ppm 1.45 (s, 9H), 3.94 (s, 3H), 7.54 (d, 1H), 7.65 (d, 1H), 7.81 (dd, 1H), 8.23 (d, 1H).

### Preparation 26

### Methyl 5-(6-amino-3-chloro-5-fluoropyridin-2-yl)-2-chlorobenzoate

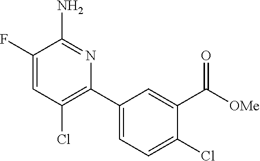 [SMILES: COC(=O)c1cc(-c2nc(N)c(F)cc2Cl)ccc1Cl]

A mixture of methyl 5-(6-amino-5-fluoropyridin-2-yl)-2-chlorobenzoate (Preparation 27, 875 mg, 3.12 mmol) and N-chlorosuccinimide (437 mg, 3.27 mmol) in acetonitrile (30 mL) was stirred at 75° C. for 16 hours. After cooling to room temperature water (50 mL) was added to and the resulting precipitate filtered to afford the title compound (880 mg, 90%).

¹H NMR (400 MHz, CDCl₃): δ ppm 3.96 (s, 3H), 4.69 (br s, 2H), 7.37 (d, 1H), 7.51 (d, 1H), 7.74 (dd, 1H), 8.17 (d, 1H).

LCMS Rt=3.33 minutes MS m/z 315 [M+H]⁺

### Preparation 27

### Methyl 5-(6-amino-5-fluoropyridin-2-yl)-2-chlorobenzoate

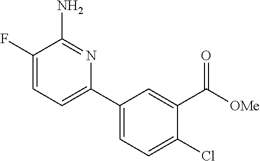 [SMILES: COC(=O)c1cc(-c2ccc(F)c(N)n2)ccc1Cl]

To a degassed solution of (4-chloro-3-(methoxycarbonyl)phenyl)boronic acid (1.67 g, 7.79 mmol), 6-chloro-3-fluoropyridin-2-amine (1.51 g, 8.18 mmol), potassium carbonate (3.25 g, 23.4 mmol) and water (20 mL) in dioxane (95 mL) was added [1,1′-bis(diphenylphosphino)ferrocene] dichloropalladium(II) (615 mg, 0.779 mmol). The reaction was stirred at 100° C. for 2 hours. The reaction was cooled and diluted with water (50 mL) and ethyl acetate (50 mL). The aqueous layer was extracted with ethyl acetate (2×80 mL), and the combined organic layers were washed with brine (100 mL), dried over MgSO₄ and concentrated in vacuo. The residue was purified using silica gel column chromatography eluting with 20% ethyl acetate in heptanes to afford the title compound (875 mg, 40%).

¹H NMR (400 MHz, CDCl₃): δ ppm 3.96 (s, 3H), 4.68 (br s, 2H), 7.06 (dd, 1H), 7.28 (m, 1H), 7.49 (d, 1H), 7.95 (dd, 1H), 8.36 (d, 1H).

### Preparation 28

### Methyl 5-(6-((tert-butoxycarbonyl)amino)pyridin-2-yl)-2,4-dichlorobenzoate

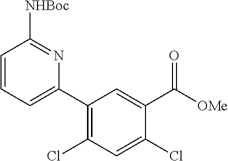 [SMILES: COC(=O)c1cc(-c2cccc(NC(=O)OC(C)(C)C)n2)c(Cl)cc1Cl]

The title compound was prepared according to the method described for Preparation 27 using methyl 2,4-dichloro-5-(4,4,5,5-tetramethyl-1,3,2-dioxaborolan-2-yl)benzoate and tert-butyl (6-bromopyridin-2-yl)carbamate with Pd(PPh₃)₄. The residue was purified using silica gel column chromatography eluting with 10-15% TBME in heptanes.

¹H NMR (400 MHz, CDCl₃): δ ppm 1.55 (s, 9H), 3.93 (s, 3H), 7.23-7.33 (m, 2H), 7.60 (s, 1H), 7.75 (t, 1H), 7.96 (d, 1H), 8.07 (s, 1H).

### Preparation 29

### 2-(phenyldiazenyl)succinonitrile

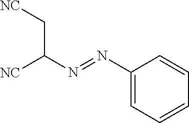 [SMILES: N#CCC(C#N)/N=N/c1ccccc1]

A solution of ethyl 2,3-dicyanopropanoate (1.58 g, 10.38 mmol) in methanol (52 mL) and pyridine (2.52 mL, 31.14 mmol) was cooled to 10° C. with an ice bath. Benzenediazonium tetrafluoroborate was added in portions over 20 minutes at 10° C. and the reaction stirred at room temperature for 18 hours. The reaction was concentrated in vacuo and partitioned between DCM and water. The organic layer was dried over magnesium sulphate and concentrated in vacuo to afford the title compound (2.8 g, 28%).

¹H NMR (400 MHz, CDCl₃): δ ppm 3.62 (s, 2H), 7.05-7.25 (m, 3H), 7.36 (m, 2H).

### Preparation 30

### 6-amino-2-chloro-5-fluoronicotinonitrile

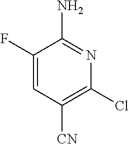 [SMILES: N#Cc1cc(F)c(N)nc1Cl]

A suspension of 2,6-dichloro-5-fluoronicotinonitrile (1.0 g, 5.24 mmol) in aqueous ammonia (28-30%, 20 mL) was heated to 120° C. in a sealed vessel for 4 hours. The resulting precipitate was filtered and triturated with MeOH to afford the title compound (530 mg, 59%).

¹H NMR (400 MHz, DMSO-d₆): δ ppm 7.84 (br s, 2H), 7.98 (d, 1H).

MS m/z 170 [M−H]⁻

### Preparation 31

### Methyl 5-(6-((tert-butoxycarbonyl)amino)pyridin-2-yl)-2-chloro-4-fluorobenzoate

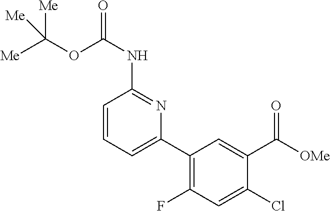 [SMILES: COC(=O)c1cc(-c2cccc(NC(=O)OC(C)(C)C)n2)c(F)cc1Cl]

The title compound was prepared according to the method described for Preparation 27 using methyl 2-chloro-4-fluoro-5-(4,4,5,5-tetramethyl-1,3,2-dioxaborolan-2-yl)benzoate and tert-butyl (6-bromopyridin-2-yl)carbamate with Pd(PPh₃)₄ at 110° C. for 1 hour. The residue was purified using silica gel column chromatography eluting with 0-2% methanol in dichloromethane to give a colourless oil (70%).

¹H NMR (400 MHz, CDCl₃): δ ppm 1.54 (s, 9H), 3.96 (s, 3H), 7.31 (d, 1H), 7.35 (br s, 1H), 7.48 (dd, 1H), 7.74 (t, 1H), 7.94 (d, 1H), 8.56 (d, 1H).

### Preparation 32

### 5-(6-((tert-butoxycarbonyl)amino)pyridin-2-yl)-2-chloro-4-fluorobenzoic Acid

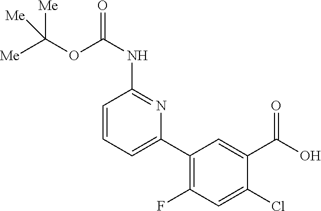 [SMILES: CC(C)(C)OC(=O)Nc1cccc(-c2cc(C(=O)O)c(Cl)cc2F)n1]

The title compound was prepared according to the method described for Preparation 23 using methyl 5-(6-((tert-butoxycarbonyl)amino)pyridin-2-yl)-2-chloro-4-fluorobenzoate (Preparation 31) in dioxane for 2 hours. The residue was isolated by extraction with dichloromethane to give a colourless solid (93%).

¹H NMR (400 MHz, CDCl₃): δ ppm 1.55 (s, 9H), 7.31 (d, 1H), 7.44 (dd, 1H), 7.77 (t, 1H), 7.99 (d, 1H), 8.20 (br s, 1H), 8.66 (d, 1H).

LCMS Rt=3.27 minutes, m/z=365.21 [M−H]⁻

### Preparation 33

### Ethyl 5-(5-(6-((tert-butoxycarbonyl)amino)pyridin-2-yl)-2-chloro-4-fluorobenzamido)-1-phenyl-1H-pyrazole-3-carboxylate

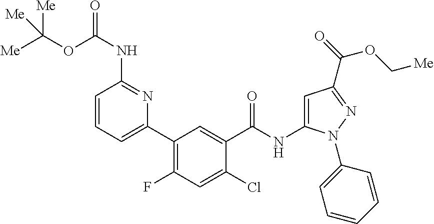 [SMILES: CCOC(=O)c1cc(NC(=O)c2cc(-c3cccc(NC(=O)OC(C)(C)C)n3)c(F)cc2Cl)n(-c2ccccc2)n1]

A solution of 5-(6-((tert-butoxycarbonyl)amino)pyridin-2-yl)-2-chloro-4-fluorobenzoic acid (Preparation 32, 500 mg, 1.36 mmol), ethyl 5-amino-1-phenyl-1H-pyrazole-3-carboxylate (315 mg, 1.36 mmol) and pyridine (323 mg, 4.09 mmol) in 2-methyltetrahydrofuran (20 mL) was heated under nitrogen to 80° C. To the solution was added T3P (1.3 g, 2.0 mmol) and the reaction heated at 80° C. for 17 hours. Additional T3P (6.2 g, 1.0 mmol) and ethyl 5-amino-1-phenyl-1H-pyrazole-3-carboxylate (150 mg, 0.65 mmol) were added and heating continued for 24 hours. The mixture was diluted with ethyl acetate, washed with aqueous NaOH solution (2M, 20 mL), hydrochloric acid (2M, 20 mL) and saturated ammonium chloride solution (40 mL). The organic layer was concentrated in vacuo and the residue was purified using silica gel column chromatography eluting with 0-2% methanol in dichloromethane to afford the title compound as a pale yellow solid.

¹H NMR (400 MHz, CDCl₃): δ ppm 1.42 (t, 3H), 1.55 (s, 9H), 4.45 (q, 2H), 7.21 (d, 1H), 7.42 (s, 1H), 7.48-7.55 (m, 7H), 7.74 (t, 1H), 7.94 (d, 1H), 8.43 (br s, 1H), 8.62 (d, 1H).

LCMS Rt=1.85 minutes, m/z=480.11 [M+H-Boc]⁺

### Preparation 34

### Ethyl 5-(5-(6-aminopyridin-2-yl)-2-chloro-4-fluorobenzamido)-1-phenyl-1H-pyrazole-3-carboxylate Trifluoroacetate

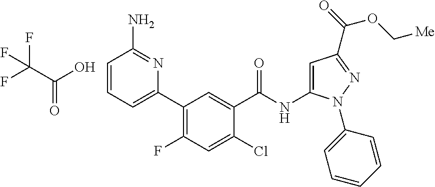

To a solution of ethyl 5-(5-(6-((tert-butoxycarbonyl)amino)pyridin-2-yl)-2-chloro-4-fluorobenzamido)-1-phenyl-1H-pyrazole-3-carboxylate (Preparation 33, 602 mg, 1.03 mmol) in dichloromethane (10 mL) was added trifluoroacetic acid (3 mL) and the mixture stirred at room temperature for 72 hours. The mixture was evaporated to dryness and azeotroped with dichloromethane. The residue was triturated with acetonitrile to give the title compound as a colourless solid (365 mg, 82%).

¹H NMR (400 MHz, MeOH-d₄): δ ppm 1.40 (t, 3H), 4.40 (q, 2H), 7.01 (d, 1H), 7.09 (d, 1H), 7.10 (s, 1H), 7.45-7.61 (m, 6H), 7.90 (d, 1H), 7.98 (dd, 1H).

LCMS Rt=2.58 minutes, m/z=480.14 [M+H]⁺

### Preparation 35

### 5-(5-(6-aminopyridin-2-yl)-2-chloro-4-fluorobenzamido)-1-phenyl-1H-pyrazole-3-carboxylic Acid

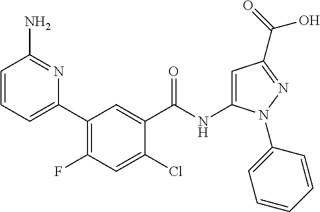 [SMILES: Nc1cccc(-c2cc(C(=O)Nc3cc(C(=O)O)nn3-c3ccccc3)c(Cl)cc2F)n1]

To a solution of ethyl 5-(5-(6-aminopyridin-2-yl)-2-chloro-4-fluorobenzamido)-1-phenyl-1H-pyrazole-3-carboxylate trifluoroacetate (Preparation 34, 365 mg, 0.67 mmol) in dioxane (10 mL) was added aqueous lithium hydroxide solution (1M, 6 mL) and the mixture stirred at room temperature for 2 hours. Hydrochloric acid (2M, 3 mL) was added and the mixture concentrated in vacuo until a precipitate formed. The mixture was filtered, washing with water, to give the title compound as a colourless solid (216 mg, 71%).

¹H NMR (400 MHz, DMSO-d₆): δ ppm 6.90 (br s, 1H), 6.93 (s, 1H), 7.01 (d, 1H), 7.45-7.60 (m, 6H), 7.70-7.90 (m, 2H), 8.00 (d, 1H), 10.87 (s, 1H).

LCMS Rt=2.08 minutes, m/z=452.09 [M+H]⁺

### Preparation 36

### 5-(5-(6-aminopyridin-2-yl)-2,4-dichlorobenzamido)-1-phenyl-1H-pyrazole-3-carboxylic Acid

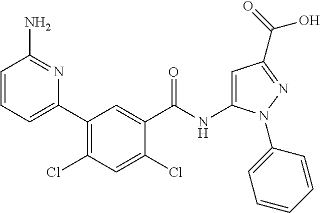 [SMILES: Nc1cccc(-c2cc(C(=O)Nc3cc(C(=O)O)nn3-c3ccccc3)c(Cl)cc2Cl)n1]

The title compound was prepared according to the method described for Preparation 35 using ethyl 5-(5-(6-aminopyridin-2-yl)-2,4-dichlorobenzamido)-1-phenyl-1H-pyrazole-3-carboxylate (Example 145) as its trifluoroacetate salt.

¹H NMR (400 MHz, DMSO-d₆): δ ppm 6.85 (br s, 1H), 6.93 (s, 1H), 7.45-7.60 (m, 7H), 7.70-7.90 (m, 2H), 7.95 (s, 1H), 10.95 (s, 1H).

LCMS Rt=2.16 minutes, m/z=468.16 [M+H]⁺

### Preparation 37

### 5-(5-(6-((tert-butoxycarbonyl)amino)pyridin-2-yl)-2,4-dichlorobenzamido)-1-phenyl-1H-pyrazole-3-carboxylic Acid

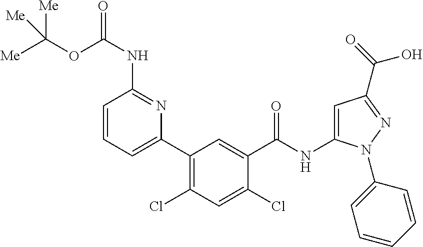 [SMILES: CC(C)(C)OC(=O)Nc1cccc(-c2cc(C(=O)Nc3cc(C(=O)O)nn3-c3ccccc3)c(Cl)cc2Cl)n1]

The title compound was prepared according to the method described for Preparation 35 using ethyl 5-(5-(6-((tert-butoxycarbonyl)amino)pyridin-2-yl)-2,4-dichlorobenzamido)-1-phenyl-1H-pyrazole-3-carboxylate (Preparation 5). The reaction was quenched with aqueous 2% citric acid solution and extracted with ethyl acetate. The organic layer was washed with water, saturated brine, dried over sodium sulphate and evaporated to give the title compound.

¹H NMR (400 MHz, DMSO-d₆): δ ppm 1.47 (s, 9H), 6.93 (s, 1H), 7.30 (d, 1H), 7.45 (m, 1H), 7.50-7.60 (m, 4H), 7.72 (s, 1H), 7.82-7.90 (m, 3H), 9.87 (s, 1H), 10.83 (s, 1H), 13.00 (br s, 1H).

LCMS Rt=2.67 minutes, m/z=566.27 [M−H]⁻

### Preparation 38

### Ethyl 5-amino-4-methyl-1-phenyl-1H-pyrazole-3-carboxylate

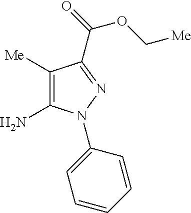 [SMILES: CCOC(=O)c1nn(-c2ccccc2)c(N)c1C]

To a solution of potassium tert-butoxide (4.0 g, 35.6 mmol) and 18-crown-6-ether (753 mg, 2.85 mmol) in THF (40 mL) was added diethyloxalate (5.21 g, 35.6 mmol) and the yellow solution heated at 60° C. for 3 hours. Propionitrile (2.5 mL, 35.6 mmol) was added dropwise and the mixture heated at 60° C. for 1 hour, then allowed to cool to room temperature overnight. The resulting precipitate was filtered and washed with diethyl ether (3×100 mL) and dried at 50° C. under vacuum to give a beige powder (5.7 g). The solid was dissolved in ethanol (80 mL) and treated with phenylhydrazine hydrochloride (4.26 g, 29.0 mmol). The yellow suspension was heated at 90° C. for 18 hours, then cooled to room temperature and concentrated in vacuo. The residue was partitioned between ethyl acetate (100 mL) and saturated aqueous sodium hydrogen carbonate solution (100 mL). The organic layer was separated and washed with saturated aqueous sodium hydrogen carbonate solution (100 mL), water (100 mL), saturated brine solution (100 mL), dried over magnesium sulphate and concentrated under reduced pressure. The residue was purified using silica gel column chromatography using 0-4% methanol in dichloromethane to give the title compound as a brown solid (3.79 g, 43%).

¹H NMR (400 MHz, CDCl₃): δ ppm 1.40 (t, 3H), 2.20 (s, 3H), 4.40 (q, 2H), 7.40 (m, 1H), 7.49 (t, 2H), 7.55 (d, 2H).

LCMS Rt=2.52 minutes, m/z=246.15 [M+H]⁺

### Biological Activity

Isolated TRK Enzyme assays use the HTRF KinEASE-TK kit (Cisbio Cat#62TK0PEJ) with recombinant His-tagged cytoplasmic domains of each TRK receptor sourced from Invitrogen (see table below). This activity-assay measures the phosphorylation of tyrosine residues within a substrate from the HTRF kit which has been validated by Cisbio for a variety of tyrosine kinases including the TRK receptors.

Assay details:

| | Invitrogen | Amino | FAC | FAC | Assay Reaction |
|---|---|---|---|---|---|
| Target | Cat# | acids | enzyme | ATP | Time |
| TRKA | PV3144 | aa 441-796 | 4 nM | 40 uM | 35 min |
| | (NTRK1) | | | | |
| TRKB | PV3616 | aa 526-838 | 1 nM | 1.4 uM | 40 min |
| | (NTRK2) | | | | |
| TRKC | PV3617 | aa 510-825 | 10 nM | 15 uM | 30 min |
| | (NTRK3) | | | | |

0.5 mM stock solutions of test compounds are prepared and serially diluted in 100% DMSO. A standard curve using the compound of Example 135 disclosed in WO2005/116035 of 150 uM is also prepared on each test plate. High percentage effect (HPE) is defined by 150 uM (using the compound of Example 135 as disclosed in WO2005/116035) and 0% effect (ZPE) is defined by 100% DMSO. Greiner low volume black plates containing 0.2 ul of serially diluted compound, standard and HPE/ZPE are created using the Bravo nanolitre dispenser.

1× enzyme buffer is prepared from 5× Enzymatic Buffer from the Cisbio KinEASE TK kit using MilliQ water. The buffer is then supplemented with 10 mM MgCl and 2 mM DTT (both from Sigma). In the case of TRKB, the buffer is also supplemented with 125 nM Supplement Enzymatic Buffer (SEB) from the Cisbio kit.

2×FAC of enzyme and 2×FAC ATP diluted in 1× complete enzyme buffer is incubated at room temperature for 20 minutes to preactivate the enzyme. Following this preactivation step, 5 ul/well of enzyme+ATP mix is added using a Multidrop Micro to the assay plate, spotted with 0.2 ul 100% DMSO compound. This is left for 20 mins at room temperature before adding 5 ul of 2 uM TK-substrate-Biotin (from the Cisbio kit) diluted in 1× enzyme buffer (1 uM FAC) using the Multidrop Micro. The reaction is incubated at room temperature for the optimized assay reaction time (see table). The reaction is stopped by adding 10 ul/well HTRF Detection Buffer containing 0.25 uM Streptavidin-XL665 (0.125 uM FAC) and 1:200 TK Antibody-Cryptate using a Multidrop.

After the Detection Reagent addition, plates are covered and incubated at room temperature for 60 minutes. HTRF signal is read using an Envision reader, measured as a ratio of emissions at two different wavelengths, 620 nm and 665 nm. Any compound that inhibits the action of the TRK kinase will have a lower fluorescence ratio value 665/620 nM than compounds which do not inhibit the TRK kinase. Test compound data are expressed as percentage inhibition defined by HPE and ZPE values for each plate. Percentage inhibition in the presence of test compound is plotted against compound concentration on a log scale to determine an IC₅₀ from the resultant sigmoid curve.

Cell Based Assays were carried out using Cell lines from DiscoveRx utilising their PathHunter technology and reagents in an antagonist assay:

| Target | DiscoveRx cell line Cat# | Cognate Neurotrophin |
|---|---|---|
| TRKA | 93-0462C3 | NGF |
| TRKA co expressed | 93-0529C3 | NGF |
| with p75 | | |
| TRKB | 93-0463C3 | BDNF |
| TRKB co expressed | 93-0530C3 | BDNF |
| with p75 | | |
| TRKC | 93-0464C3 | NT3 |
| TRKC co expressed | 93-0531C3 | NT3 |
| with p75 | | |

The assays are based upon DiscoveRx's proprietary Enzyme Fragment Complementation (EFC) technology. In the case of the TRK cell lines, the enzyme acceptor (EA) protein is fused to a SH2 protein and the TRK receptor of interest has been tagged with a Prolink tag.

Upon neurotrophin binding, the TRK receptor becomes phosphorylated, and the tagged SH2 protein binds. This results in functional complementation and restored β-Galactosidase activity which is can be measured using the luminescent Galacton Star substrate within the PathHunter reagent kits.

Generally, small molecule inhibitors bind to the kinase domain so are not competing with the neurotrophin (agonist) which binds to an extracellular site. This means that the IC₅₀ is a good measure of affinity and should be unaffected by concentration neurotrophin stimulant.

Cryopreserved PathHunter cells are used from either in-house produced batches or bulk batches bought directly from DiscoveRx. Cryopreserved cells are resuscitated, spun 1000 rpm for 4 min to remove freezing media, and resuspended in MEM+0.5% horse serum (both Invitrogen) to 5e⁵ cells/ml. The cells are then plated using a Multidrop into Greiner white tissue culture treated plates at 20 ul/well and incubated for 24 h at 37° C., 5% CO₂, high humidity. On the day of the assay, the cell plates are allowed to cool to room temperature for 30 min prior to the assay.

4 mM stock solutions of test compounds are prepared and serially diluted in 100% DMSO. A standard curve using the compound of Example 135, WO2005/116035 at a top concentration of 150 uM is also prepared on each test plate. High percentage effect (HPE) is defined by 150 uM of the compound of Example 135, WO2005/116035 and 0% effect (ZPE) is defined by 100% DMSO. Plates containing 1 ul of serially diluted compound, standard and HPE/ZPE are diluted 1/66 in assay buffer (PBS minus Ca²⁺, minus Mg²⁺ with 0.05% pluronic F127) using a Wellmate. Using a Platemate Plus, 5 ul of 1/66 diluted test compounds is then transferred to the cell plate and allowed to reach equilibrium by incubating for 30 min at room temperature before addition of agonist stimulus: 10 ul/well of 2 nM (0.571 nM FAC) of the cognate neurotrophin (Peprotech) diluted in agonist buffer (HBSS with 0.25% BSA). Final assay concentration of the test compounds is 8.66 μM, (the compound of Example 135, WO2005/116035 FAC is 0.325 uM). The plates are left at room temperature for a further 2 hours before addition of 10 ul of the DiscoveRx PathHunter detection reagent (made up by adding 1 part Galacton Star, 5 parts Emerald II and 19 parts Cell Assay Buffer as per the manufacturer's instructions).

After reagent addition, plates are covered and incubated at room temperature for 60 minutes. Luminescence signal is read using an Envision. Test compound data are expressed as percentage inhibition defined by HPE and ZPE values for each plate. Percentage inhibition in the presence of test compound is plotted against compound concentration on a log scale to determine an IC₅₀ from the resultant sigmoid curve.

### Brain Penetration Assays

### In Vitro

MDCK-BCRP: MDCK-BCRP data may be collected according to the method described in “A 96-Well Efflux Assay To Identify ABCG2 Substrates Using a Stably Transfected MDCK II Cell Line” http://pubs.acs.org/doi/full/10.1021/mp050088t

Yongling Xiao, Ralph Davidson, Arthur Smith, Dennis Pereira, Sabrina Zhao, John Soglia, David Gebhard, Sonia de Morais, and David B. Duignan, Mol. Pharm., 2006, 3 (1), pp 45-54.

MDCK-MDR1: MDCK-MDR1 data may be collected according to the method described in “Are MDCK Cells Transfected with the Human MDR1 Gene a Good Model of the Human Intestinal Mucosa?” http://www.springerlink.com/content/gfhglgbr4fnp3khf/fulltext.pdf

Fuxing Tang, Kazutoshi Horie, and Ronald T. Borchardt, Pharmaceutical Research, Vol. 19, No. 6, June 2002.

### In Vivo

Brain penetration may be measured according to the method described in “Assessing brain free fraction in early drug discovery”. Read, K; Braggio, S., Expert Opinion Drug Metab Toxicol. (2010) 6 (3) 337-344.

TrkA IC₅₀ data are illustrated below. Where more than one reading was taken, the arithmetic mean is presented.

| Example | TrkA IC₅₀ (nM) |
|---|---|
| 1 | 14.04 |
| 2 | 24.88 |
| 3 | 95.08 |
| 4 | 66.09 |
| 5 | 157.05 |
| 6 | 71.26 |
| 7 | 69.09 |
| 8 | 50.41 |
| 9 | 15.39 |
| 10 | 18.68 |
| 11 | 36.87 |
| 12 | 11.21 |
| 13 | 8.23 |
| 14 | 19.95 |
| 15 | 16.12 |
| 16 | 17.01 |
| 17 | 9.55 |
| 18 | 19.04 |
| 19 | 14.12 |
| 20 | 12.5 |
| 21 | 25.49 |
| 22 | 15.93 |
| 23 | 22.71 |
| 24 | 12.97 |
| 25 | 16.63 |
| 26 | 20.57 |
| 27 | 41.97 |
| 28 | 15.55 |
| 29 | 26.52 |
| 30 | 147.59 |
| 31 | 102.05 |
| 32 | 82.33 |
| 33 | 188.07 |
| 34 | 43.91 |
| 35 | 138.42 |
| 36 | 54.35 |
| 37 | 485 |
| 38 | 48.84 |
| 39 | 378.04 |
| 40 | 94.3 |
| 41 | 11.81 |
| 42 | 160.4 |
| 43 | 80.26 |
| 44 | 77.25 |
| 45 | 49.62 |
| 46 | 49.29 |
| 47 | 41.48 |
| 48 | 56.61 |
| 49 | 39.86 |
| 50 | 52.3 |
| 51 | 55.97 |
| 52 | 8.55 |
| 53 | 35.46 |
| 54 | 201.55 |
| 55 | 51.13 |
| 56 | 18.06 |
| 57 | 46.44 |
| 58 | 46.78 |
| 59 | 295.62 |
| 60 | 21.91 |
| 61 | 25.98 |
| 62 | 34.41 |
| 63 | 84.46 |
| 64 | 71.12 |
| 65 | 203.19 |
| 66 | 66.55 |
| 67 | 179.09 |
| 68 | 47.62 |
| 69 | 130.81 |
| 70 | 60.02 |
| 71 | 41.58 |
| 72 | 14.44 |
| 73 | 122.69 |
| 74 | 28.22 |
| 75 | 314.86 |
| 76 | 28.94 |
| 77 | 8.72 |
| 78 | 86.26 |
| 79 | 41.99 |
| 80 | 262.87 |
| 81 | 168.01 |
| 82 | 17.84 |
| 83 | 38 |
| 84 | 234.28 |
| 85 | 873.62 |
| 86 | 22.92 |
| 87 | 23.65 |
| 88 | 227.19 |
| 89 | 32.87 |
| 90 | 74.94 |
| 91 | 58.94 |
| 92 | 84 |
| 93 | 31.71 |
| 94 | 22.35 |
| 95 | 47.35 |
| 96 | 28.86 |
| 97 | 25.91 |
| 98 | 69.88 |
| 99 | 303.13 |
| 100 | 600.99 |
| 101 | 5.3 |
| 102 | 34.52 |
| 103 | 63.29 |
| 104 | 58.82 |
| 105 | 75.41 |
| 106 | 98.07 |
| 107 | 110.07 |
| 108 | 359.22 |
| 109 | 643.93 |
| 110 | 110.66 |
| 111 | 74.87 |
| 112 | 105.17 |
| 113 | 58.23 |
| 114 | 50.68 |
| 115 | 67.85 |
| 116 | 16.48 |
| 117 | 10.72 |
| 118 | 20.77 |
| 119 | 24.39 |
| 120 | 7.88 |
| 121 | 20.32 |
| 122 | 23.76 |
| 123 | 82.28 |
| 124 | 1.99 |
| 125 | 296.51 |
| 126 | 69.97 |
| 127 | 15.67 |
| 128 | 239.09 |
| 129 | 32.84 |
| 130 | 54.86 |
| 131 | 15.85 |
| 132 | 1445.26 |
| 133 | 166.93 |
| 134 | 1002.48 |
| 135 | 14.84 |
| 137 | 687.66 |
| 153 | 6.05 |
| 154 | 6.13 |
| 155 | 6.95 |
| 156 | 3.05 |
| 157 | 1.55 |
| 158 | 927 |
| 159 | 171 |

All publications cited in this application are each herein incorporated by reference in their entirety.

Although the invention has been described above with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

1. A compound of Formula I 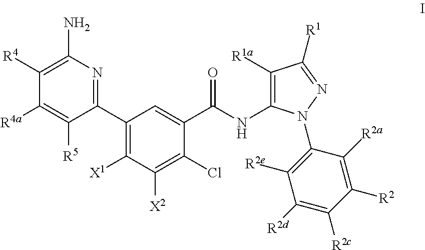
Or a prodrug thereof, or a pharmaceutically acceptable salt thereof, wherein
R¹ is CON(H or C₁₋₆ alkyl optionally substituted by 1 or 2 substituents independently selected from F, OH and OMe)(H or C₁₋₆ alkyl optionally substituted by 1, 2 or 3 substituents independently selected from F, NH₂, OH and OMe), CONR^{x1}(C₃₋₆ cycloalkyl optionally substituted by 1, 2 or 3 substituents independently selected from OH, NH₂, CH₃ and CH₂OH), CONR^{x1}(CR^{Y}R^{X})ₘ(C₃₋₆ cycloalkyl optionally substituted by 1, 2 or 3 substituents independently selected from OH, NH₂, CH₃ and CH₂OH), CONR^{x1}-Het, CO—NHet, CONR^{x1}(CR^{Y}R^{X})ₘ—CON(H or C₁₋₄ alkyl optionally substituted by 1 or 2 substituents independently selected from F, OH and OMe)(H or C₁₋₄ alkyl optionally substituted by 1, 2 or 3 substituents independently selected from F, OH and OMe), CONR^{x1}(CR^{Y}R^{X})ₘ—N(H or C₁₋₄ alkyl optionally substituted by 1 or 2 substituents independently selected from F, OH and OMe)(H or C₁₋₄ alkyl optionally substituted by 1, 2 or 3 substituents independently selected from F, OH and OMe), CONR^{x1}(CR^{Y}R^{X})ₘN(H or C₁₋₄ alkyl optionally substituted by 1 or 2 substituents independently selected from F, OH and OMe)CO(C₁₋₄ alkyl optionally substituted by 1, 2 or 3 substituents independently selected from F, OH and OMe), CONR^{x1}C(O)-Het, C(O)NR^{x1}(CR^{Y}R^{X})ₘ-Het, CONR^{x1}—Ar, C(O)—NR^{x1}-Het, CN, CO₂H, or CO₂(C₁₋₄ alkyl optionally substituted by 1 or 2 substituents independently selected from F, OH and OMe),
m is an integer from 1 to 3,
Ar is phenyl optionally substituted by 1, 2 or 3 groups independently selected from C₁₋₆ alkyl, halogen, CN, CF₃, CF₃O, C₁₋₆ alkoxy, C₁₋₆ alkoxy-O—C(O)—, CONH₂, C₁₋₆ alkylthio, hydroxy-C₁₋₆ alkyl, C₁₋₆ alkyl-SO₂—, CO₂H and C₁₋₃ alkoxy-C₁₋₃ alkyl-O—C(O)—,
Het is a 4-7-membered saturated or unsaturated heterocyclic ring having at least 1, and up to 3, hetero ring atoms independently selected from N, O and S, and which ring is optionally substituted by 1, 2 or 3 substituents independently selected from halogen, OH, ═O, CN, CONH₂, O(C₁₋₆ alkyl optionally substituted by one or more F), C(O)(C₁₋₆ alkyl optionally substituted by one or more F), C₁₋₆ alkyl optionally substituted by one or more F, C₁₋₆ alkyl substituted by CN, C₁₋₆ alkyl substituted by up to 3 OH, C₁₋₆ alkyl substituted by CO₂(C₁₋₄ alkyl), C₁₋₆ alkyl substituted by one or more C₁₋₃ alkoxy, S(O)ₚ(C₁₋₆ alkyl optionally substituted by one or more F), CO₂(C₁₋₆ alkyl), C₃₋₆ cycloalkyl, C(O)(C₃₋₆ cycloalkyl), N(H or C₁₋₃ alkyl)CO(C₁₋₃ alkyl) and N(H or C₁₋₃ alkyl)(H or C₁₋₃ alkyl),
NHet is a 4-7-membered saturated or unsaturated heterocyclic ring with a ring N atom directly linked to the C(O) moiety, having from 0 to 2 further hetero ring atoms independently selected from N, O and S, and which ring is optionally substituted by up to 3 substituents independently selected from halogen, OH, ═O, CN, CONH₂, C₁₋₆ alkyl optionally substituted by one or more F, O(C₁₋₆ alkyl optionally substituted by one or more F), C(O)(C₁₋₆ alkyl optionally substituted by one or more F), C₁₋₆ alkyl substituted by CN, C₁₋₆ alkyl substituted by up to 3 OH, C₁₋₆ alkyl substituted by CO₂(C₁₋₄ alkyl), C₁₋₆ alkyl substituted by one or more C₁₋₃ alkoxy, S(O)ₚ(C₁₋₆ alkyl optionally substituted by one or more F), CO₂(C₁₋₆ alkyl), C₃₋₆ cycloalkyl, C(O)(C₃₋₆ cycloalkyl), N(H or C₁₋₃ alkyl)CO(C₁₋₃ alkyl), and N(H or C₁₋₃ alkyl)(H or C₁₋₃ alkyl),
R^{2a}, R², R^{2c}, R^{2d} and R^{2e} are each independently selected from H, OH, halogen, NH₂, or methyl optionally substituted by up to 3 F,
X¹, X², R^{1a}, R⁴, R^{4a} and R⁵ are each independently selected from H, C₃₋₆ cycloalkyl, C₀₋₆ alkyl optionally substituted by up to 3 substituents independently selected from halogen, CN, CO₂H, OH, (C₁₋₆ alkoxy optionally substituted by up to 3 F), S(O)ₚ(C₁₋₆ alkyl optionally substituted by up to 3 F), C(O)(C₁₋₆ alkoxy optionally substituted by up to 3 F or by C₁₋₃ alkoxy), C(O)NR^{x1}R^{x2}, NR^{x1}R^{x2}, O(C₃₋₆ cycloalkyl), Ar, Het, CO₂(C₁₋₆ alkyl optionally substituted by up to 3 F), NR^{x1}C(O)(C₁₋₆ alkyl optionally substituted by up to 3 F), NR^{x1}C(O)NR^{x2}(C₁₋₆ alkyl optionally substituted by up to 3 F), OC(O)(C₁₋₆ alkyl optionally substituted by up to 3 F), and OC(O)NR^{x1}R^{x2},
p is 0, 1 or 2,
R^{x1} and R^{x2} are each independently H, C₁₋₃ alkyl optionally substituted by up to 3 substituents independently selected from F, OH and OCH₃, or, together with the nitrogen atom to which they are attached, form a 4- to 6-membered saturated ring,
and R^{x} and R^{y} are each independently H, C₁₋₃ alkoxy optionally substituted by up to 3 F, C₁₋₃ alkyl optionally substituted by up to 3 substituents independently selected from F, OH and OCH₃, or, together with the carbon atom to which they are attached, are C₃₋₆ cycloalkyl.

2. The compound, prodrug, or salt according to claim 1 wherein R^{1a} is H and R^{4a} is H.

3. A The compound, prodrug, or salt according to claim 1 wherein R^{2a}, R², R^{2c}, R^{2d} and R^{2e} are each independently selected from H, F and OH.

4. The compound, prodrug, or salt according to claim 1 wherein R^{2a}, R^{2c}, R^{2d} and R^{2e} are each H and R² is H or OH.

5. The compound according to claim 1 which is of Formula I″ 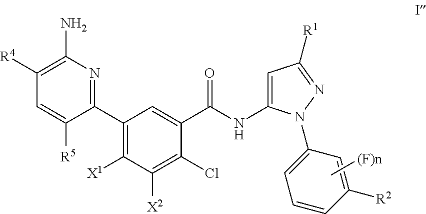
or a prodrug, or a pharmaceutically acceptable salt thereof, wherein
n is an integer from 0 to 4,
R¹ is CON(H or C₁₋₄ alkyl optionally substituted by 1 or 2 substituents independently selected from F, OH and OMe)(H or C₁₋₄ alkyl optionally substituted by 1, 2 or 3 substituents independently selected from F, NH₂, OH and OMe), CONH(C₃₋₆ cycloalkyl optionally substituted by 1, 2 or 3 substituents independently selected from OH, NH₂, CH₃ and CH₂OH), CONH(CR^{Y}R^{X})ₘ(C₃₋₆ cycloalkyl optionally substituted by 1, 2 or 3 substituents independently selected from OH, NH₂, CH₃ and CH₂OH), CONH-Het, CONH(CR^{Y}R^{X})m-CON(H or C₁₋₄ alkyl optionally substituted by 1 or 2 substituents independently selected from F, OH and OMe)(H or C₁₋₄ alkyl optionally substituted by 1, 2 or 3 substituents independently selected from F, OH and OMe), CONH(CR^{Y}R^{X})ₘ—N(H or C₁₋₄ alkyl optionally substituted by 1 or 2 substituents independently selected from F, OH and OMe)(H or C₁₋₄ alkyl optionally substituted by 1, 2 or 3 substituents independently selected from F, OH and OMe), CONH(CR^{Y}R^{X})ₘN(H or C₁₋₄ alkyl optionally substituted by 1 or 2 substituents independently selected from F, OH and OMe)CO(C₁₋₄ alkyl optionally substituted by 1, 2 or 3 substituents independently selected from F, OH and OMe), CONHC(O)-Het, C(O)NH(CR^{Y}R^{X})ₘ-Het, CONH—Ar, C(O)—NH-Het, CN, CO₂H, and CO₂(C₁₋₄ alkyl optionally substituted by 1 or 2 substituents independently selected from F, OH and OMe),
m is an integer from 1 to 3,
Ar is phenyl optionally substituted by 1, 2 or 3 groups independently selected from C₁₋₆ alkyl, halogen, CN, CF₃, CF₃O, C₁₋₆ alkoxy, C₁₋₆ alkoxy-O—C(O)—, CONH₂, C₁₋₆ alkylthio, hydroxy-C₁₋₆ alkyl, C₁₋₆ alkyl-SO₂—, CO₂H and C₁₋₃ alkoxy-C₁₋₃ alkyl-O—C(O)—,
Het is a 4-7-membered saturated or unsaturated heterocyclic ring having at least 1, and up to 3, hetero ring atoms independently selected from N, O and S, and which ring is optionally substituted by 1, 2 or 3 substituents independently selected from halogen, OH, ═O, CN, CONH₂, O(C₁₋₆ alkyl optionally substituted by one or more F), C(O)(C₁₋₆ alkyl optionally substituted by one or more F), C₁₋₆ alkyl optionally substituted by one or more F, C₁₋₆ alkyl substituted by CN, C₁₋₆ alkyl substituted by up to 3 OH, C₁₋₆ alkyl optionally substituted by CO₂(C₁₋₄ alkyl), C₁₋₆ alkyl substituted by one or more C₁₋₃ alkoxy, SO₂(C₁₋₆ alkyl optionally substituted by one or more F), CO₂(C₁₋₆ alkyl), C₃₋₆ cycloalkyl, C(O)(C₃₋₆ cycloalkyl) and N(H or C₁₋₃ alkyl)CO(C₁₋₃ alkyl),
NHet is a 4-7-membered saturated or unsaturated heterocyclic ring with a ring N atom directly linked to the C(O) moiety to which it is attached, and having from 0 to 2 further hetero ring atoms independently selected from N, O and S, and which ring is optionally substituted by up to 3 substituents independently selected from halogen, OH, ═O, CN, CONH₂, C₁₋₆ alkyl optionally substituted by one or more F, O(C₁₋₆ alkyl optionally substituted by one or more F), C(O)(C₁₋₆ alkyl optionally substituted by one or more F), C₁₋₆ alkyl substituted by CN, C₁₋₆ alkyl substituted by up to 3 OH, C₁₋₆ alkyl optionally substituted by CO₂(C₁₋₄ alkyl), C₁₋₆ alkyl substituted by one or more C₁₋₃ alkoxy, SO₂(C₁₋₆ alkyl optionally substituted by one or more F), CO₂(C₁₋₆ alkyl), C₃₋₆ cycloalkyl, C(O)(C₃₋₆ cycloalkyl), and N(H or C₁₋₃ alkyl)CO(C₁₋₃ alkyl),
R² is H or OH,
X¹ is H, Cl, F, CN, C₁₋₃ alkyl optionally substituted by one or more F, C₁₋₃ alkoxy optionally substituted by one or more F, or cyclopropyl,
X² is H, Cl, F, CN, C₁₋₃ alkyl optionally substituted by one or more F, C₁₋₃ alkoxy optionally substituted by one or more F, or cyclopropyl,
R⁴ is H, F, Cl, CN, C₁₋₃ alkyl optionally substituted by one or more F, C₁₋₃ alkoxy optionally substituted by one or more F, or cyclopropyl,
R⁵ is H, Cl, F, CN, C₁₋₃ alkyl optionally substituted by one or more F, C₁₋₃ alkoxy optionally substituted by one or more F, or cyclopropyl,
and R^{x} and R^{y} are each independently H or C₁₋₃ alkyl.

6. The compound, prodrug, or salt according to claim 1 wherein R¹ is selected from CON(H or C₁₋₄ alkyl optionally substituted by 1 or 2 substituents independently selected from F, OH and OMe)(H or C₁₋₄ alkyl optionally substituted by 1, 2 or 3 substituents independently selected from F, NH₂, OH and OMe), CONH(C₃₋₆ cycloalkyl optionally substituted by 1, 2 or 3 substituents independently selected from OH, NH₂, CH₃ and CH₂OH), CONH-Het, and C(O)NH(CR^{Y}R^{X})ₘ-Het.

7. The compound, prodrug, or salt according to claim 1 wherein R¹ is selected from CONH(H, CH₃ or C₂₋₄ alkyl optionally substituted by F, NH₂, OH or OMe), CONH(C₃₋₆ cycloalkyl optionally substituted by 1, 2 or 3 substituents independently selected from OH, CH₃ and CH₂OH), CONH-Het, and C(O)NH(CR^{Y}R^{X})ₘ-Het1, where Het1 is a 5- or 6-membered unsaturated heterocyclic ring having from 1 to 3 N ring atoms, and which ring is optionally substituted by up to 3 substituents independently selected from C₁₋₆ alkyl optionally substituted by one or more F.

8. The compound, prodrug, or salt according to claim 1 wherein R¹ is selected from CONH(H, CH₃ or C₂₋₄ alkyl optionally substituted by OH), CONH(C₃₋₆ cycloalkyl optionally substituted by 1, 2 or 3 substituents independently selected from OH, CH₃ and CH₂OH), CONH-Het, and C(O)NH(CR^{Y}R^{X})ₘ-Het1, where Het1 is a 5- or 6-membered unsaturated heterocyclic ring having from 1 to 3 N ring atoms, and which ring is optionally substituted by up to 3 substituents independently selected from C₁₋₆ alkyl optionally substituted by one or more F.

9. The compound, prodrug, or salt according to 1 claim wherein R¹ is selected from CONH(pyrazolyl or 1,2,3-triazolyl optionally substituted by 1 or 2 methyl groups; 2-methyl-2-hydroxypropyl or 2-hydroxyethyl).

10. The compound, prodrug, or salt according to claim 1 R¹ is selected from

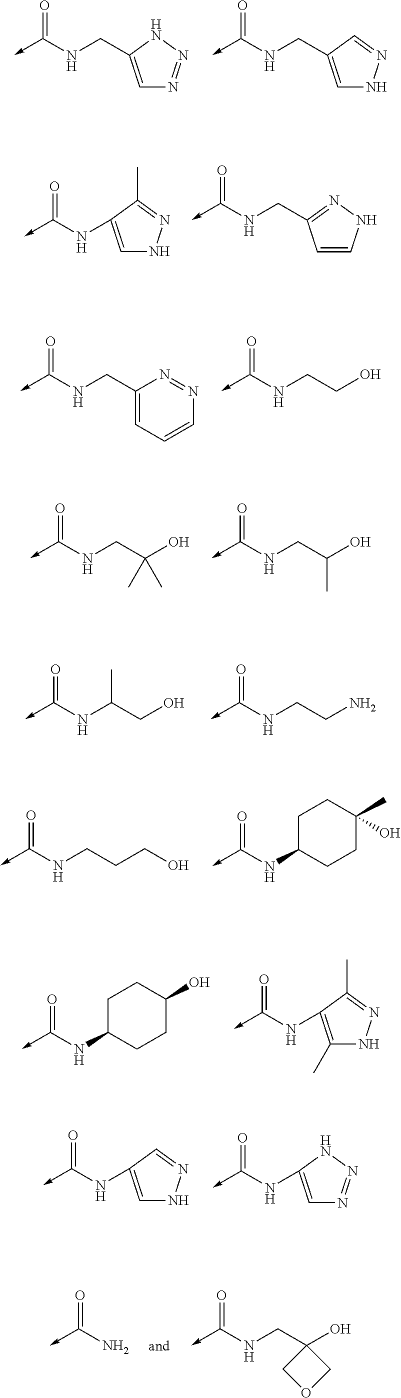

11. The compound, prodrug, or salt according to claim 1 wherein X¹ is H, F or Cl.

12. The compound, prodrug, or salt according to claim 1 wherein X² is H.

13. The compound, prodrug, or salt according to claim 1 wherein R⁴ is H, F, Cl, CH₃, CN or OCH₃.

14. The compound, prodrug, or salt according to claim 1 wherein R⁵ is H, F, Cl, CH₃, CN or OCH₃.

15. A compound of formula I′″ 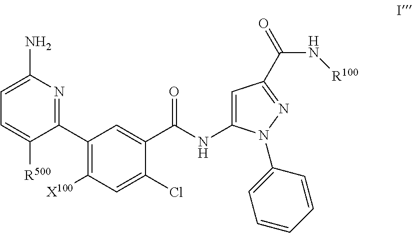
Wherein
R⁵⁰⁰ is H or F,
X¹⁰⁰ is H, F or Cl,
And R¹⁰⁰ is pyrazol-4-yl optionally substituted by 1 or 2 methyl groups, 1,2,3-triazol-4-yl optionally substituted by methyl, 2-hydroxyethyl, or 2-methyl-2-hydroxypropyl, or prodrug, or pharmaceutically acceptable salt thereof.

16. The pharmaceutical composition comprising a compound, prodrug, or a pharmaceutically acceptable salt thereof, as defined in claim 1, and a pharmaceutically acceptable carrier.

17-20. (canceled)

21. The method of treatment of a mammal, to treat a disease or condition for which a TrkA receptor antagonist is indicated, comprising treating said mammal with an effective amount of a compound, prodrug, or a pharmaceutically acceptable salt thereof, as defined in claim 1.

22. The method of claim 21 wherein the disease or condition is pain or cancer.

23. (canceled)
